SEQUENCE LISTING (478 sequences)

SEQ ID NO: 1 - RNA, 991 nt, Homo sapiens
  source 1..991: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatg

SEQ ID NO: 2 - RNA, 991 nt, Homo sapiens
  source 1..991: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaagcatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatg

SEQ ID NO: 3 - RNA, 320 nt, Homo sapiens
  source 1..320: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggaca

SEQ ID NO: 4 - RNA, 320 nt, Homo sapiens
  source 1..320: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaagcatggatggagagatgggtgagtacatggatggatagatggatgagttggtgggtagattcgtggctagatggatgatggatggatggaca

SEQ ID NO: 5 - RNA, 128 nt, Homo sapiens
  source 1..128: mol_type = mRNA
ctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 6 - RNA, 178 nt, Homo sapiens
  source 1..178: mol_type = mRNA
ccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 7 - RNA, 279 nt, Homo sapiens
  source 1..279: mol_type = mRNA
tgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctc

SEQ ID NO: 8 - RNA, 227 nt, Homo sapiens
  source 1..227: mol_type = mRNA
gactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctc

SEQ ID NO: 9 - RNA, 173 nt, Homo sapiens
  source 1..173: mol_type = mRNA
tattaatattatcactatgcttactgtgccatatagtaccggatacgggatgaagtcatacaagcactgaatgaatggatgaatgaatgatggatgaatggatgacaccttcttatatgtgtatcaggctgatgctgaagacttcaaagttgagtaaaatacctatgtcagtc

SEQ ID NO: 10 - RNA, 112 nt, Homo sapiens
  source 1..112: mol_type = mRNA
taccggatacgggatgaagtcatacaagcactgaatgaatggatgaatgaatgatggatgaatggatgacaccttcttatatgtgtatcaggctgatgctgaagacttcaaa

SEQ ID NO: 11 - RNA, 40 nt, Homo sapiens
  source 1..40: mol_type = mRNA
taaaattatattcatattgcaggactcggcagaagacctt

SEQ ID NO: 12 - RNA, 67 nt, Homo sapiens
  source 1..67: mol_type = mRNA
gtagaaaataagaatttggctctctgtgtgagcatgtgtgcgtgtgtgcgagagagagagacagaca

SEQ ID NO: 13 - RNA, 23 nt, Homo sapiens
  source 1..23: mol_type = mRNA
tgtgtgagcatgtgtgcgtgtgt

SEQ ID NO: 14 - RNA, 47 nt, Homo sapiens
  source 1..47: mol_type = mRNA
attcatattgcaggactcggcagaagaccttcgagagaaaggtagaa

SEQ ID NO: 15 - RNA, 51 nt, Homo sapiens
  source 1..51: mol_type = mRNA
taagaagaaatgaatgtgaatgcggcttgtggcacagttgacaaggatgat

SEQ ID NO: 16 - RNA, 47 nt, Homo sapiens
  source 1..47: mol_type = mRNA
aatgcggcttgtggcacagttgacaaggatgataaatcaataatgca

SEQ ID NO: 17 - RNA, 24 nt, Homo sapiens
  source 1..24: mol_type = mRNA
tgcggcttgtggcacagttgacaa

SEQ ID NO: 18 - RNA, 65 nt, Homo sapiens
  source 1..65: mol_type = mRNA
taagaagaaatgaatgtgaatgcggcttgtggcacagttgacaaggatgataaatcaataatgca

SEQ ID NO: 19 - RNA, 40 nt, Homo sapiens
  source 1..40: mol_type = mRNA
ggttcttccttctattccagccctaaccactcaggattgg

SEQ ID NO: 20 - RNA, 40 nt, Homo sapiens
  source 1..40: mol_type = mRNA
tgtctgggtatgtctcttccagctgcctgggtttcctgga

SEQ ID NO: 21 - RNA, 40 nt, Homo sapiens
  source 1..40: mol_type = mRNA
aaggatggatggagagatgggtgagtacatggatggatag

SEQ ID NO: 22 - RNA, 40 nt, Homo sapiens
  source 1..40: mol_type = mRNA
aagcatggatggagagatgggtgagtacatggatggatag

SEQ ID NO: 23 - RNA, 811 nt, Homo sapiens
  source 1..811: mol_type = mRNA
tgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatg

SEQ ID NO: 24 - RNA, 108 nt, Homo sapiens
  source 1..108: mol_type = mRNA
gtttgttgaataaatgctggtgaatgaatgaatgattgaacagatgaatgagtgatgagtagataaaaggatggatggagagatgggtgagtacatggatggatagat

SEQ ID NO: 25 - RNA, 108 nt, Homo sapiens
  source 1..108: mol_type = mRNA
gtttgttgaataaatgctggtgaatgaatgaatgattgaacagatgaatgagtgatgagtagataaaagcatggatggagagatgggtgagtacatggatggatagat

SEQ ID NO: 26 - RNA, 22 nt, Homo sapiens
  source 1..22: mol_type = mRNA
ggatggatggagagatgggtga

SEQ ID NO: 27 - RNA, 48 nt, Homo sapiens
  source 1..48: mol_type = mRNA
cttctattccagccctaaccactcaggattgggccgtttgtgtctggg

SEQ ID NO: 28 - RNA, 48 nt, Homo sapiens
  source 1..48: mol_type = mRNA
tgtctcttccagctgcctgggtttcctggaaagaactcttatccccag

SEQ ID NO: 29 - RNA, 46 nt, Homo sapiens
  source 1..46: mol_type = mRNA
ccactcaggattgggccgtttgtgtctgggtatgtctcttccagct

SEQ ID NO: 30 - RNA, 58 nt, Homo sapiens
  source 1..58: mol_type = mRNA
actcaggattgggccgtttgtgtctgggtatgtctcttccagctgcctgggtttcctg

SEQ ID NO: 31 - RNA, 40 nt, Homo sapiens
  source 1..40: mol_type = mRNA
ctatgcttactgtgccatatagtaccggatacgggatgaa

SEQ ID NO: 32 - RNA, 127 nt, Homo sapiens
  source 1..127: mol_type = mRNA
ggatacgggatgaagtcatacaagcactgaatgaatggatgaatgaatgatggatgaatggatgacaccttcttatatgtatcaggctgatgctgaagacggatacgggatgaagtcatacaagcac

SEQ ID NO: 33 - RNA, 53 nt, Homo sapiens
  source 1..53: mol_type = mRNA
tgaatgaatggatgaatgaatgatggatgaatggatgacaccttcttatatgt

SEQ ID NO: 34 - RNA, 47 nt, Homo sapiens
  source 1..47: mol_type = mRNA
tgaatgatggatgaatggatgacaccttcttatatgtgtatcaggct

SEQ ID NO: 35 - RNA, 47 nt, Homo sapiens
  source 1..47: mol_type = mRNA
atatgtgtatcaggctgatgctgaagacttcaaagttgagtaaaata

SEQ ID NO: 36 - RNA, 48 nt, Homo sapiens
  source 1..48: mol_type = mRNA
cactgaatgaatggatgaatgaatgatggatgaatggatgacaccttc

SEQ ID NO: 37 - RNA, 55 nt, Homo sapiens
  source 1..55: mol_type = mRNA
accttcttatatgtgtatcaggctgatgctgaagacttcaaagttgagtaaaata

SEQ ID NO: 38 - RNA, 49 nt, Homo sapiens
  source 1..49: mol_type = mRNA
gtaccggatacgggatgaagtcatacaagcactgaatgaatggatgaat

SEQ ID NO: 39 - RNA, 46 nt, Homo sapiens
  source 1..46: mol_type = mRNA
ctgtgccatatagtaccggatacgggatgaagtcatacaagcactg

SEQ ID NO: 40 - RNA, 46 nt, Homo sapiens
  source 1..46: mol_type = mRNA
atggatgacaccttcttatatgtgtatcaggctgatgctgaagact

SEQ ID NO: 41 - RNA, 252 nt, synthetic construct
  source 1..252: mol_type = other RNA
aacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgc

SEQ ID NO: 42 - RNA, 16 nt, synthetic construct
  source 1..16: mol_type = other RNA
gtcttctgccgagtcc

SEQ ID NO: 43 - RNA, 16 nt, synthetic construct
  source 1..16: mol_type = other RNA
gtcttctgccgagtcc

SEQ ID NO: 44 - RNA, 16 nt, synthetic construct
  source 1..16: mol_type = other RNA
tcttctgccgagtcct

SEQ ID NO: 45 - RNA, 16 nt, synthetic construct
  source 1..16: mol_type = other RNA
cttctgccgagtcctg

SEQ ID NO: 46 - RNA, 16 nt, synthetic construct
  source 1..16: mol_type = other RNA
ttctgccgagtcctgc

SEQ ID NO: 47 - RNA, 16 nt, synthetic construct
  source 1..16: mol_type = other RNA
tctgccgagtcctgca

SEQ ID NO: 48 - RNA, 16 nt, synthetic construct
  source 1..16: mol_type = other RNA
ctgccgagtcctgcaa

SEQ ID NO: 49 - RNA, 16 nt, synthetic construct
  source 1..16: mol_type = other RNA
tgccgagtcctgcaat

SEQ ID NO: 50 - RNA, 16 nt, synthetic construct
  source 1..16: mol_type = other RNA
gccgagtcctgcaata

SEQ ID NO: 51 - RNA, 16 nt, synthetic construct
  source 1..16: mol_type = other RNA
ccgagtcctgcaatat

SEQ ID NO: 52 - RNA, 16 nt, synthetic construct
  source 1..16: mol_type = other RNA
cgagtcctgcaatatg

SEQ ID NO: 53 - RNA, 16 nt, synthetic construct
  source 1..16: mol_type = other RNA
gagtcctgcaatatga

SEQ ID NO: 54 - RNA, 16 nt, synthetic construct
  source 1..16: mol_type = other RNA
agtcctgcaatatgaa

SEQ ID NO: 55 - RNA, 16 nt, synthetic construct
  source 1..16: mol_type = other RNA
gccctgcaatatgaat

SEQ ID NO: 56 - RNA, 16 nt, synthetic construct
  source 1..16: mol_type = other RNA
ccctgcaatatgaata

SEQ ID NO: 57 - RNA, 16 nt, synthetic construct
  source 1..16: mol_type = other RNA
cctgcaatatgaatat

SEQ ID NO: 58 - RNA, 16 nt, synthetic construct
  source 1..16: mol_type = other RNA
ctgcaatatgaatata

SEQ ID NO: 59 - RNA, 16 nt, synthetic construct
  source 1..16: mol_type = other RNA
tgcaatatgaatataa

SEQ ID NO: 60 - RNA, 16 nt, synthetic construct
  source 1..16: mol_type = other RNA
tgtctgtctctctctc

SEQ ID NO: 61 - RNA, 16 nt, synthetic construct
  source 1..16: mol_type = other RNA
gtctgtctctctctct

SEQ ID NO: 62 - RNA, 16 nt, synthetic construct
  source 1..16: mol_type = other RNA
tctgtctctctctctc

SEQ ID NO: 63 - RNA, 16 nt, synthetic construct
  source 1..16: mol_type = other RNA
ctgtctctctctctcg

SEQ ID NO: 64 - RNA, 16 nt, synthetic construct
  source 1..16: mol_type = other RNA
tgtctctctctctcgc

SEQ ID NO: 65 - RNA, 16 nt, synthetic construct
  source 1..16: mol_type = other RNA
gtctctctctctcgca

SEQ ID NO: 66 - RNA, 16 nt, synthetic construct
  source 1..16: mol_type = other RNA
tctctctctctcgcac

SEQ ID NO: 67 - RNA, 16 nt, synthetic construct
  source 1..16: mol_type = other RNA
ctctctctctcgcaca

SEQ ID NO: 68 - RNA, 16 nt, synthetic construct
  source 1..16: mol_type = other RNA
tctctctctcgcacac

SEQ ID NO: 69 - RNA, 16 nt, synthetic construct
  source 1..16: mol_type = other RNA
ctctctctcgcacaca

SEQ ID NO: 70 - RNA, 16 nt, synthetic construct
  source 1..16: mol_type = other RNA
tctctctcgcacacac

SEQ ID NO: 71 - RNA, 16 nt, synthetic construct
  source 1..16: mol_type = other RNA
tctctctcgcacacac

SEQ ID NO: 72 - RNA, 16 nt, synthetic construct
  source 1..16: mol_type = other RNA
tctctcgcacacacgc

SEQ ID NO: 73 - RNA, 16 nt, synthetic construct
  source 1..16: mol_type = other RNA
ctctcgcacacacgca

SEQ ID NO: 74 - RNA, 16 nt, synthetic construct
  source 1..16: mol_type = other RNA
tctcgcacacacgcac

SEQ ID NO: 75 - RNA, 16 nt, synthetic construct
  source 1..16: mol_type = other RNA
ctcgcacacacgcaca

SEQ ID NO: 76 - RNA, 16 nt, synthetic construct
  source 1..16: mol_type = other RNA
tcgcacacacgcacac

SEQ ID NO: 77 - RNA, 16 nt, synthetic construct
  source 1..16: mol_type = other RNA
cgcacacacgcacaca

SEQ ID NO: 78 - RNA, 16 nt, synthetic construct
  source 1..16: mol_type = other RNA
gcacacacgcacacat

SEQ ID NO: 79 - RNA, 16 nt, synthetic construct
  source 1..16: mol_type = other RNA
cacacacgcacacatg

SEQ ID NO: 80 - RNA, 16 nt, synthetic construct
  source 1..16: mol_type = other RNA
cacacacgcacacatg

SEQ ID NO: 81 - RNA, 16 nt, synthetic construct
  source 1..16: mol_type = other RNA
cacacgcacacatgct

SEQ ID NO: 82 - RNA, 16 nt, synthetic construct
  source 1..16: mol_type = other RNA
acacgcacacatgctc

SEQ ID NO: 83 - RNA, 16 nt, synthetic construct
  source 1..16: mol_type = other RNA
cacgcacacatgctca

SEQ ID NO: 84 - RNA, 16 nt, synthetic construct
  source 1..16: mol_type = other RNA
acgcacacatgctcac

SEQ ID NO: 85 - RNA, 16 nt, synthetic construct
  source 1..16: mol_type = other RNA
cgcacacatgctcaca

SEQ ID NO: 86 - RNA, 16 nt, synthetic construct
  source 1..16: mol_type = other RNA
gcacacatgctcacac

SEQ ID NO: 87 - RNA, 16 nt, synthetic construct
  source 1..16: mol_type = other RNA
cacacatgctcacaca

SEQ ID NO: 88 - RNA, 16 nt, synthetic construct
  source 1..16: mol_type = other RNA
acacatgctcacacag

SEQ ID NO: 89 - RNA, 16 nt, synthetic construct
  source 1..16: mol_type = other RNA
cacatgctcacacaga

SEQ ID NO: 90 - RNA, 16 nt, synthetic construct
  source 1..16: mol_type = other RNA
acatgctcacacagag

SEQ ID NO: 91 - RNA, 16 nt, synthetic construct
  source 1..16: mol_type = other RNA
catgctcacacagaga

SEQ ID NO: 92 - RNA, 16 nt, synthetic construct
  source 1..16: mol_type = other RNA
atgctcacacagagag

SEQ ID NO: 93 - RNA, 16 nt, synthetic construct
  source 1..16: mol_type = other RNA
tgctcacacagagagc

SEQ ID NO: 94 - RNA, 16 nt, synthetic construct
  source 1..16: mol_type = other RNA
gctcacacagagagcc

SEQ ID NO: 95 - RNA, 16 nt, synthetic construct
  source 1..16: mol_type = other RNA
ctcacacagagagcca

SEQ ID NO: 96 - RNA, 16 nt, synthetic construct
  source 1..16: mol_type = other RNA
tcacacagagagccaa

SEQ ID NO: 97 - RNA, 16 nt, synthetic construct
  source 1..16: mol_type = other RNA
cacacagagagccaaa

SEQ ID NO: 98 - RNA, 16 nt, synthetic construct
  source 1..16: mol_type = other RNA
acacagagagccaaat

SEQ ID NO: 99 - RNA, 16 nt, synthetic construct
  source 1..16: mol_type = other RNA
cacagagagccaaatt

SEQ ID NO: 100 - RNA, 16 nt, synthetic construct
  source 1..16: mol_type = other RNA
acagagagccaaattc

SEQ ID NO: 101 - RNA, 16 nt, synthetic construct
  source 1..16: mol_type = other RNA
cagagagccaaattct

SEQ ID NO: 102 - RNA, 16 nt, synthetic construct
  source 1..16: mol_type = other RNA
cagagagccaaattct

SEQ ID NO: 103 - RNA, 16 nt, synthetic construct
  source 1..16: mol_type = other RNA
cctgagtggttagggc

SEQ ID NO: 104 - RNA, 16 nt, synthetic construct
  source 1..16: mol_type = other RNA
ctgagtggttagggct

SEQ ID NO: 105 - RNA, 15 nt, synthetic construct
  source 1..15: mol_type = other RNA
tagtggttagggctg

SEQ ID NO: 106 - RNA, 15 nt, synthetic construct
  source 1..15: mol_type = other RNA
agtggttagggctgg

SEQ ID NO: 107 - RNA, 15 nt, synthetic construct
  source 1..15: mol_type = other RNA
gtggttagggctgga

SEQ ID NO: 108 - RNA, 15 nt, synthetic construct
  source 1..15: mol_type = other RNA
tggttagggctggaa

SEQ ID NO: 109 - RNA, 15 nt, synthetic construct
  source 1..15: mol_type = other RNA
tggttagggctggaa

SEQ ID NO: 110 - RNA, 15 nt, synthetic construct
  source 1..15: mol_type = other RNA
gttagggctggaata

SEQ ID NO: 111 - RNA, 15 nt, synthetic construct
  source 1..15: mol_type = other RNA
ttagggctggaatag

SEQ ID NO: 112 - RNA, 15 nt, synthetic construct
  source 1..15: mol_type = other RNA
tagggctggaataga

SEQ ID NO: 113 - RNA, 15 nt, synthetic construct
  source 1..15: mol_type = other RNA
agggctggaatagaa

SEQ ID NO: 114 - RNA, 15 nt, synthetic construct
  source 1..15: mol_type = other RNA
gggctggaatagaag

SEQ ID NO: 115 - RNA, 15 nt, synthetic construct
  source 1..15: mol_type = other RNA
ggctggaatagaagg

SEQ ID NO: 116 - RNA, 16 nt, synthetic construct
  source 1..16: mol_type = other RNA
gctggaatagaaggaa

SEQ ID NO: 117 - RNA, 16 nt, synthetic construct
  source 1..16: mol_type = other RNA
ctggaatagaaggaat

SEQ ID NO: 118 - RNA, 17 nt, synthetic construct
  source 1..17: mol_type = other RNA
ctggaatagaaggaata

SEQ ID NO: 119 - RNA, 16 nt, synthetic construct
  source 1..16: mol_type = other RNA
aggaaacccaggcagc

SEQ ID NO: 120 - RNA, 16 nt, synthetic construct
  source 1..16: mol_type = other RNA
ggaaacccaggcagct

SEQ ID NO: 121 - RNA, 16 nt, synthetic construct
  source 1..16: mol_type = other RNA
gaaacccaggcagctg

SEQ ID NO: 122 - RNA, 16 nt, synthetic construct
  source 1..16: mol_type = other RNA
aaacccaggcagctgg

SEQ ID NO: 123 - RNA, 16 nt, synthetic construct
  source 1..16: mol_type = other RNA
aacccaggcagctgga

SEQ ID NO: 124 - RNA, 16 nt, synthetic construct
  source 1..16: mol_type = other RNA
acccaggcagctggaa

SEQ ID NO: 125 - RNA, 16 nt, synthetic construct
  source 1..16: mol_type = other RNA
cccaggcagctggaag

SEQ ID NO: 126 - RNA, 16 nt, synthetic construct
  source 1..16: mol_type = other RNA
ccaggcagctggaaga

SEQ ID NO: 127 - RNA, 16 nt, synthetic construct
  source 1..16: mol_type = other RNA
caggcagctggaagag

SEQ ID NO: 128 - RNA, 16 nt, synthetic construct
  source 1..16: mol_type = other RNA
aggcagctggaagaga

SEQ ID NO: 129 - RNA, 16 nt, synthetic construct
  source 1..16: mol_type = other RNA
aggcagctggaagaga

SEQ ID NO: 130 - RNA, 16 nt, synthetic construct
  source 1..16: mol_type = other RNA
gcagctggaagagaca

SEQ ID NO: 131 - RNA, 16 nt, synthetic construct
  source 1..16: mol_type = other RNA
cagctggaagagacat

SEQ ID NO: 132 - RNA, 16 nt, synthetic construct
  source 1..16: mol_type = other RNA
agctggaagagacata

SEQ ID NO: 133 - RNA, 16 nt, synthetic construct
  source 1..16: mol_type = other RNA
gctggaagagacatac

SEQ ID NO: 134 - RNA, 16 nt, synthetic construct
  source 1..16: mol_type = other RNA
ctggaagagacatacc

SEQ ID NO: 135 - RNA, 16 nt, synthetic construct
  source 1..16: mol_type = other RNA
tggaagagacataccc

SEQ ID NO: 136 - RNA, 16 nt, synthetic construct
  source 1..16: mol_type = other RNA
tccatccatgtactca

SEQ ID NO: 137 - RNA, 16 nt, synthetic construct
  source 1..16: mol_type = other RNA
ccatccatgtactcac

SEQ ID NO: 138 - RNA, 16 nt, synthetic construct
  source 1..16: mol_type = other RNA
ccatccatgtactcac

SEQ ID NO: 139 - RNA, 16 nt, synthetic construct
  source 1..16: mol_type = other RNA
atccatgtactcaccc

SEQ ID NO: 140 - RNA, 16 nt, synthetic construct
  source 1..16: mol_type = other RNA
atccatgtactcaccc

SEQ ID NO: 141 - RNA, 16 nt, synthetic construct
  source 1..16: mol_type = other RNA
ccatgtactcacccat

SEQ ID NO: 142 - RNA, 16 nt, synthetic construct
  source 1..16: mol_type = other RNA
catgtactcacccatc

SEQ ID NO: 143 - RNA, 16 nt, synthetic construct
  source 1..16: mol_type = other RNA
atgtactcacccatct

SEQ ID NO: 144 - RNA, 16 nt, synthetic construct
  source 1..16: mol_type = other RNA
tgtactcacccatctc

SEQ ID NO: 145 - RNA, 16 nt, synthetic construct
  source 1..16: mol_type = other RNA
gtactcacccatctct

SEQ ID NO: 146 - RNA, 16 nt, synthetic construct
  source 1..16: mol_type = other RNA
tactcacccatctctc

SEQ ID NO: 147 - RNA, 16 nt, synthetic construct
  source 1..16: mol_type = other RNA
actcacccatctctcc

SEQ ID NO: 148 - RNA, 16 nt, synthetic construct
  source 1..16: mol_type = other RNA
ctcacccatctctcca

SEQ ID NO: 149 - RNA, 16 nt, synthetic construct
  source 1..16: mol_type = other RNA
tcacccatctctccat

SEQ ID NO: 150 - RNA, 16 nt, synthetic construct
  source 1..16: mol_type = other RNA
cacccatctctccatc

SEQ ID NO: 151 - RNA, 16 nt, synthetic construct
  source 1..16: mol_type = other RNA
cacccatctctccatc

SEQ ID NO: 152 - RNA, 16 nt, synthetic construct
  source 1..16: mol_type = other RNA
cccatctctccatcca

SEQ ID NO: 153 - RNA, 16 nt, synthetic construct
  source 1..16: mol_type = other RNA
atctatccatccatgt

SEQ ID NO: 154 - RNA, 16 nt, synthetic construct
  source 1..16: mol_type = other RNA
tctatccatccatgta

SEQ ID NO: 155 - RNA, 16 nt, synthetic construct
  source 1..16: mol_type = other RNA
ctatccatccatgtac

SEQ ID NO: 156 - RNA, 16 nt, synthetic construct
  source 1..16: mol_type = other RNA
tatccatccatgtact

SEQ ID NO: 157 - RNA, 16 nt, synthetic construct
  source 1..16: mol_type = other RNA
atccatccatgtactc

SEQ ID NO: 158 - RNA, 16 nt, synthetic construct
  source 1..16: mol_type = other RNA
tccatccatgtactca

SEQ ID NO: 159 - RNA, 16 nt, synthetic construct
  source 1..16: mol_type = other RNA
ccatccatgtactcac

SEQ ID NO: 160 - RNA, 16 nt, synthetic construct
  source 1..16: mol_type = other RNA
catccatgtactcacc

SEQ ID NO: 161 - RNA, 16 nt, synthetic construct
  source 1..16: mol_type = other RNA
atccatgtactcaccc

SEQ ID NO: 162 - RNA, 16 nt, synthetic construct
  source 1..16: mol_type = other RNA
tccatgtactcaccca

SEQ ID NO: 163 - RNA, 16 nt, synthetic construct
  source 1..16: mol_type = other RNA
tccatgtactcaccca

SEQ ID NO: 164 - RNA, 15 nt, synthetic construct
  source 1..15: mol_type = other RNA
atgtactcacccatc

SEQ ID NO: 165 - RNA, 15 nt, synthetic construct
  source 1..15: mol_type = other RNA
tgtactcacccatct

SEQ ID NO: 166 - RNA, 15 nt, synthetic construct
  source 1..15: mol_type = other RNA
gtactcacccatctc

SEQ ID NO: 167 - RNA, 15 nt, synthetic construct
  source 1..15: mol_type = other RNA
tactcacccatctct

SEQ ID NO: 168 - RNA, 15 nt, synthetic construct
  source 1..15: mol_type = other RNA
actcacccatctctc

SEQ ID NO: 169 - RNA, 15 nt, synthetic construct
  source 1..15: mol_type = other RNA
ctcacccatctctcc

SEQ ID NO: 170 - RNA, 15 nt, synthetic construct
  source 1..15: mol_type = other RNA
tcacccatctctcca

SEQ ID NO: 171 - RNA, 15 nt, synthetic construct
  source 1..15: mol_type = other RNA
cacccatctctccat

SEQ ID NO: 172 - RNA, 15 nt, synthetic construct
  source 1..15: mol_type = other RNA
acccatctctccatc

SEQ ID NO: 173 - RNA, 15 nt, synthetic construct
  source 1..15: mol_type = other RNA
cccatctctccatcc

SEQ ID NO: 174 - RNA, 15 nt, synthetic construct
  source 1..15: mol_type = other RNA
ccatctctccatcca

SEQ ID NO: 175 - RNA, 15 nt, synthetic construct
  source 1..15: mol_type = other RNA
catctctccatccat

SEQ ID NO: 176 - RNA, 15 nt, synthetic construct
  source 1..15: mol_type = other RNA
atctctccatccatc

SEQ ID NO: 177 - RNA, 15 nt, synthetic construct
  source 1..15: mol_type = other RNA
atctctccatccatg

SEQ ID NO: 178 - RNA, 15 nt, synthetic construct
  source 1..15: mol_type = other RNA
tctctccatccatcc

SEQ ID NO: 179 - RNA, 15 nt, synthetic construct
  source 1..15: mol_type = other RNA
tctctccatccatgc

SEQ ID NO: 180 - RNA, 15 nt, synthetic construct
  source 1..15: mol_type = other RNA
ctctccatccatcct

SEQ ID NO: 181 - RNA, 15 nt, synthetic construct
  source 1..15: mol_type = other RNA
ctctccatccatgct

SEQ ID NO: 182 - RNA, 15 nt, synthetic construct
  source 1..15: mol_type = other RNA
tctccatccatcctt

SEQ ID NO: 183 - RNA, 15 nt, synthetic construct
  source 1..15: mol_type = other RNA
tctccatccatgctt

SEQ ID NO: 184 - RNA, 15 nt, synthetic construct
  source 1..15: mol_type = other RNA
ctccatccatccttt

SEQ ID NO: 185 - RNA, 15 nt, synthetic construct
  source 1..15: mol_type = other RNA
ctccatccatgcttt

SEQ ID NO: 186 - RNA, 15 nt, synthetic construct
  source 1..15: mol_type = other RNA
tccatccatcctttt

SEQ ID NO: 187 - RNA, 15 nt, synthetic construct
  source 1..15: mol_type = other RNA
tccatccatgctttt

SEQ ID NO: 188 - RNA, 15 nt, synthetic construct
  source 1..15: mol_type = other RNA
ccatccatcctttta

SEQ ID NO: 189 - RNA, 15 nt, synthetic construct
  source 1..15: mol_type = other RNA
ccatccatgctttta

SEQ ID NO: 190 - RNA, 15 nt, synthetic construct
  source 1..15: mol_type = other RNA
catccatccttttat

SEQ ID NO: 191 - RNA, 15 nt, synthetic construct
  source 1..15: mol_type = other RNA
catccatgcttttat

SEQ ID NO: 192 - RNA, 15 nt, synthetic construct
  source 1..15: mol_type = other RNA
atccatccttttatc

SEQ ID NO: 193 - RNA, 15 nt, synthetic construct
  source 1..15: mol_type = other RNA
atccatgcttttatc

SEQ ID NO: 194 - RNA, 15 nt, synthetic construct
  source 1..15: mol_type = other RNA
tccatccttttatct

SEQ ID NO: 195 - RNA, 15 nt, synthetic construct
  source 1..15: mol_type = other RNA
tccatgcttttatct

SEQ ID NO: 196 - RNA, 15 nt, synthetic construct
  source 1..15: mol_type = other RNA
ccatccttttatcta

SEQ ID NO: 197 - RNA, 15 nt, synthetic construct
  source 1..15: mol_type = other RNA
ccatgcttttatcta

SEQ ID NO: 198 - RNA, 15 nt, synthetic construct
  source 1..15: mol_type = other RNA
catccttttatctac

SEQ ID NO: 199 - RNA, 15 nt, synthetic construct
  source 1..15: mol_type = other RNA
catgcttttatctac

SEQ ID NO: 200 - RNA, 15 nt, synthetic construct
  source 1..15: mol_type = other RNA
atccttttatctact

SEQ ID NO: 201 - RNA, 15 nt, synthetic construct
  source 1..15: mol_type = other RNA
atgcttttatctact

SEQ ID NO: 202 - RNA, 15 nt, synthetic construct
  source 1..15: mol_type = other RNA
tccttttatctactc

SEQ ID NO: 203 - RNA, 15 nt, synthetic construct
  source 1..15: mol_type = other RNA
tgcttttatctactc

SEQ ID NO: 204 - RNA, 15 nt, synthetic construct
  source 1..15: mol_type = other RNA
ccttttatctactca

SEQ ID NO: 205 - RNA, 15 nt, synthetic construct
  source 1..15: mol_type = other RNA
gcttttatctactca

SEQ ID NO: 206 - RNA, 15 nt, synthetic construct
  source 1..15: mol_type = other RNA
cttttatctactcat

SEQ ID NO: 207 - RNA, 15 nt, synthetic construct
  source 1..15: mol_type = other RNA
ttttatctactcatc

SEQ ID NO: 208 - RNA, 15 nt, synthetic construct
  source 1..15: mol_type = other RNA
tttatctactcatca

SEQ ID NO: 209 - RNA, 15 nt, synthetic construct
  source 1..15: mol_type = other RNA
ttatctactcatcac

SEQ ID NO: 210 - RNA, 15 nt, synthetic construct
  source 1..15: mol_type = other RNA
tatctactcatcact

SEQ ID NO: 211 - RNA, 15 nt, synthetic construct
  source 1..15: mol_type = other RNA
atctactcatcactc

SEQ ID NO: 212 - RNA, 15 nt, synthetic construct
  source 1..15: mol_type = other RNA
tctactcatcactca

SEQ ID NO: 213 - RNA, 15 nt, synthetic construct
  source 1..15: mol_type = other RNA
ctactcatcactcat

SEQ ID NO: 214 - RNA, 15 nt, synthetic construct
  source 1..15: mol_type = other RNA
tactcatcactcatt

SEQ ID NO: 215 - RNA, 15 nt, synthetic construct
  source 1..15: mol_type = other RNA
actcatcactcattc

SEQ ID NO: 216 - RNA, 15 nt, synthetic construct
  source 1..15: mol_type = other RNA
ctcatcactcattca

SEQ ID NO: 217 - RNA, 15 nt, synthetic construct
  source 1..15: mol_type = other RNA
tcatcactcattcat

SEQ ID NO: 218 - RNA, 15 nt, synthetic construct
  source 1..15: mol_type = other RNA
catcactcattcatc

SEQ ID NO: 219 - RNA, 15 nt, synthetic construct
  source 1..15: mol_type = other RNA
atcactcattcatct

SEQ ID NO: 220 - RNA, 15 nt, synthetic construct
  source 1..15: mol_type = other RNA
tcactcattcatctg

SEQ ID NO: 221 - RNA, 15 nt, synthetic construct
  source 1..15: mol_type = other RNA
cactcattcatctgt

SEQ ID NO: 222 - RNA, 15 nt, synthetic construct
  source 1..15: mol_type = other RNA
actcattcatctgtt

SEQ ID NO: 223 - RNA, 15 nt, synthetic construct
  source 1..15: mol_type = other RNA
ctcattcatctgttc

SEQ ID NO: 224 - RNA, 15 nt, synthetic construct
  source 1..15: mol_type = other RNA
tcattcatctgttca

SEQ ID NO: 225 - RNA, 15 nt, synthetic construct
  source 1..15: mol_type = other RNA
cattcatctgttcaa

SEQ ID NO: 226 - RNA, 15 nt, synthetic construct
  source 1..15: mol_type = other RNA
attcatctgttcaat

SEQ ID NO: 227 - RNA, 15 nt, synthetic construct
  source 1..15: mol_type = other RNA
ttcatctgttcaatc

SEQ ID NO: 228 - RNA, 15 nt, synthetic construct
  source 1..15: mol_type = other RNA
tcatctgttcaatca

SEQ ID NO: 229 - RNA, 15 nt, synthetic construct
  source 1..15: mol_type = other RNA
catctgttcaatcat

SEQ ID NO: 230 - RNA, 15 nt, synthetic construct
  source 1..15: mol_type = other RNA
atctgttcaatcatt

SEQ ID NO: 231 - RNA, 15 nt, synthetic construct
  source 1..15: mol_type = other RNA
tctgttcaatcattc

SEQ ID NO: 232 - RNA, 15 nt, synthetic construct
  source 1..15: mol_type = other RNA
ctgttcaatcattca

SEQ ID NO: 233 - RNA, 15 nt, synthetic construct
  source 1..15: mol_type = other RNA
tgttcaatcattcat

SEQ ID NO: 234 - RNA, 15 nt, synthetic construct
  source 1..15: mol_type = other RNA
gttcaatcattcatt

SEQ ID NO: 235 - RNA, 15 nt, synthetic construct
  source 1..15: mol_type = other RNA
ttcaatcattcattc

SEQ ID NO: 236 - RNA, 15 nt, synthetic construct
  source 1..15: mol_type = other RNA
tcaatcattcattca

SEQ ID NO: 237 - RNA, 15 nt, synthetic construct
  source 1..15: mol_type = other RNA
caatcattcattcat

SEQ ID NO: 238 - RNA, 15 nt, synthetic construct
  source 1..15: mol_type = other RNA
aatcattcattcatt

SEQ ID NO: 239 - RNA, 15 nt, synthetic construct
  source 1..15: mol_type = other RNA
atcattcattcattc

SEQ ID NO: 240 - RNA, 15 nt, synthetic construct
  source 1..15: mol_type = other RNA
tcattcattcattca

SEQ ID NO: 241 - RNA, 15 nt, synthetic construct
  source 1..15: mol_type = other RNA
cattcattcattcac

SEQ ID NO: 242 - RNA, 15 nt, synthetic construct
  source 1..15: mol_type = other RNA
attcattcattcacc

SEQ ID NO: 243 - RNA, 15 nt, synthetic construct
  source 1..15: mol_type = other RNA
ttcattcattcacca

SEQ ID NO: 244 - RNA, 15 nt, synthetic construct
  source 1..15: mol_type = other RNA
tcattcattcaccag

SEQ ID NO: 245 - RNA, 15 nt, synthetic construct
  source 1..15: mol_type = other RNA
cattcattcaccagc

SEQ ID NO: 246 - RNA, 15 nt, synthetic construct
  source 1..15: mol_type = other RNA
attcattcaccagca

SEQ ID NO: 247 - RNA, 15 nt, synthetic construct
  source 1..15: mol_type = other RNA
ttcattcaccagcat

SEQ ID NO: 248 - RNA, 15 nt, synthetic construct
  source 1..15: mol_type = other RNA
tcattcaccagcatt

SEQ ID NO: 249 - RNA, 15 nt, synthetic construct
  source 1..15: mol_type = other RNA
cattcaccagcattt

SEQ ID NO: 250 - RNA, 15 nt, synthetic construct
  source 1..15: mol_type = other RNA
attcaccagcattta

SEQ ID NO: 251 - RNA, 15 nt, synthetic construct
  source 1..15: mol_type = other RNA
ttcaccagcatttat

SEQ ID NO: 252 - RNA, 15 nt, synthetic construct
  source 1..15: mol_type = other RNA
tcaccagcatttatt

SEQ ID NO: 253 - RNA, 15 nt, synthetic construct
  source 1..15: mol_type = other RNA
caccagcatttattc

SEQ ID NO: 254 - RNA, 15 nt, synthetic construct
  source 1..15: mol_type = other RNA
accagcatttattca

SEQ ID NO: 255 - RNA, 15 nt, synthetic construct
  source 1..15: mol_type = other RNA
ccagcatttattcaa

SEQ ID NO: 256 - RNA, 15 nt, synthetic construct
  source 1..15: mol_type = other RNA
cagcatttattcaac

SEQ ID NO: 257 - RNA, 15 nt, synthetic construct
  source 1..15: mol_type = other RNA
agcatttattcaaca

SEQ ID NO: 258 - RNA, 15 nt, synthetic construct
  source 1..15: mol_type = other RNA
gcatttattcaacaa

SEQ ID NO: 259 - RNA, 15 nt, synthetic construct
  source 1..15: mol_type = other RNA
catttattcaacaaa

SEQ ID NO: 260 - RNA, 15 nt, synthetic construct
  source 1..15: mol_type = other RNA
atttattcaacaaac

SEQ ID NO: 261 - RNA, 16 nt, synthetic construct
  source 1..16: mol_type = other RNA
atcccgtatccggtac

SEQ ID NO: 262 - RNA, 16 nt, synthetic construct
  source 1..16: mol_type = other RNA
tcccgtatccggtact

SEQ ID NO: 263 - RNA, 16 nt, synthetic construct
  source 1..16: mol_type = other RNA
cccgtatccggtacta

SEQ ID NO: 264 - RNA, 16 nt, synthetic construct
  source 1..16: mol_type = other RNA
ccgtatccggtactat

SEQ ID NO: 265 - RNA, 16 nt, synthetic construct
  source 1..16: mol_type = other RNA
cgtatccggtactata

SEQ ID NO: 266 - RNA, 16 nt, synthetic construct
  source 1..16: mol_type = other RNA
gtatccggtactatat

SEQ ID NO: 267 - RNA, 16 nt, synthetic construct
  source 1..16: mol_type = other RNA
tatccggtactatatg

SEQ ID NO: 268 - RNA, 16 nt, synthetic construct
  source 1..16: mol_type = other RNA
atccggtactatatgg

SEQ ID NO: 269 - RNA, 16 nt, synthetic construct
  source 1..16: mol_type = other RNA
tccggtactatatggc

SEQ ID NO: 270 - RNA, 16 nt, synthetic construct
  source 1..16: mol_type = other RNA
ccggtactatatggca

SEQ ID NO: 271 - RNA, 16 nt, synthetic construct
  source 1..16: mol_type = other RNA
cggtactatatggcac

SEQ ID NO: 272 - RNA, 16 nt, synthetic construct
  source 1..16: mol_type = other RNA
cggtactatatggcac

SEQ ID NO: 273 - RNA, 16 nt, synthetic construct
  source 1..16: mol_type = other RNA
atactatatggcacag

SEQ ID NO: 274 - RNA, 16 nt, synthetic construct
  source 1..16: mol_type = other RNA
tactatatggcacagt

SEQ ID NO: 275 - RNA, 16 nt, synthetic construct
  source 1..16: mol_type = other RNA
actatatggcacagta

SEQ ID NO: 276 - RNA, 16 nt, synthetic construct
  source 1..16: mol_type = other RNA
ctatatggcacagtaa

SEQ ID NO: 277 - RNA, 16 nt, synthetic construct
  source 1..16: mol_type = other RNA
tatatggcacagtaag

SEQ ID NO: 278 - RNA, 16 nt, synthetic construct
  source 1..16: mol_type = other RNA
gtcttcagcatcagcc

SEQ ID NO: 279 - RNA, 16 nt, synthetic construct
  source 1..16: mol_type = other RNA
tcttcagcatcagcct

SEQ ID NO: 280 - RNA, 16 nt, synthetic construct
  source 1..16: mol_type = other RNA
cttcagcatcagcctg

SEQ ID NO: 281 - RNA, 16 nt, synthetic construct
  source 1..16: mol_type = other RNA
ttcagcatcagcctga

SEQ ID NO: 282 - RNA, 16 nt, synthetic construct
  source 1..16: mol_type = other RNA
tcagcatcagcctgat

SEQ ID NO: 283 - RNA, 16 nt, synthetic construct
  source 1..16: mol_type = other RNA
cagcatcagcctgata

SEQ ID NO: 284 - RNA, 16 nt, synthetic construct
  source 1..16: mol_type = other RNA
agcatcagcctgatac

SEQ ID NO: 285 - RNA, 16 nt, synthetic construct
  source 1..16: mol_type = other RNA
gcatcagcctgataca

SEQ ID NO: 286 - RNA, 16 nt, synthetic construct
  source 1..16: mol_type = other RNA
catcagcctgatacat

SEQ ID NO: 287 - RNA, 16 nt, synthetic construct
  source 1..16: mol_type = other RNA
atcagcctgatacata

SEQ ID NO: 288 - RNA, 16 nt, synthetic construct
  source 1..16: mol_type = other RNA
tcagcctgatacatat

SEQ ID NO: 289 - RNA, 16 nt, synthetic construct
  source 1..16: mol_type = other RNA
cagcctgatacatata

SEQ ID NO: 290 - RNA, 16 nt, synthetic construct
  source 1..16: mol_type = other RNA
agcctgatacatataa

SEQ ID NO: 291 - RNA, 16 nt, synthetic construct
  source 1..16: mol_type = other RNA
gcctgatacatataag

SEQ ID NO: 292 - RNA, 16 nt, synthetic construct
  source 1..16: mol_type = other RNA
cctgatacatataaga

SEQ ID NO: 293 - RNA, 16 nt, synthetic construct
  source 1..16: mol_type = other RNA
ctgatacatataagaa

SEQ ID NO: 294 - RNA, 16 nt, synthetic construct
  source 1..16: mol_type = other RNA
tgatacatataagaag

SEQ ID NO: 295 - RNA, 16 nt, synthetic construct
  source 1..16: mol_type = other RNA
gatacatataagaagg

SEQ ID NO: 296 - RNA, 16 nt, synthetic construct
  source 1..16: mol_type = other RNA
atacatataagaaggt

SEQ ID NO: 297 - RNA, 16 nt, synthetic construct
  source 1..16: mol_type = other RNA
tacatataagaaggtg

SEQ ID NO: 298 - RNA, 15 nt, synthetic construct
  source 1..15: mol_type = other RNA
acatataagaaggtg

SEQ ID NO: 299 - RNA, 15 nt, synthetic construct
  source 1..15: mol_type = other RNA
catataagaaggtgt

SEQ ID NO: 300 - RNA, 16 nt, synthetic construct
  source 1..16: mol_type = other RNA
acatataagaaggtgt

SEQ ID NO: 301 - RNA, 16 nt, synthetic construct
  source 1..16: mol_type = other RNA
catataagaaggtgtc

SEQ ID NO: 302 - RNA, 16 nt, synthetic construct
  source 1..16: mol_type = other RNA
atataagaaggtgtca

SEQ ID NO: 303 - RNA, 16 nt, synthetic construct
  source 1..16: mol_type = other RNA
tataagaaggtgtcat

SEQ ID NO: 304 - RNA, 16 nt, synthetic construct
  source 1..16: mol_type = other RNA
ataagaaggtgtcatc

SEQ ID NO: 305 - RNA, 16 nt, synthetic construct
  source 1..16: mol_type = other RNA
taagaaggtgtcatcc

SEQ ID NO: 306 - RNA, 16 nt, synthetic construct
  source 1..16: mol_type = other RNA
aagaaggtgtcatcca

SEQ ID NO: 307 - RNA, 16 nt, synthetic construct
  source 1..16: mol_type = other RNA
agaaggtgtcatccat

SEQ ID NO: 308 - RNA, 16 nt, synthetic construct
  source 1..16: mol_type = other RNA
gaaggtgtcatccatt

SEQ ID NO: 309 - RNA, 16 nt, synthetic construct
  source 1..16: mol_type = other RNA
aaggtgtcatccattc

SEQ ID NO: 310 - RNA, 16 nt, synthetic construct
  source 1..16: mol_type = other RNA
aggtgtcatccattca

SEQ ID NO: 311 - RNA, 16 nt, synthetic construct
  source 1..16: mol_type = other RNA
ggtgtcatccattcat

SEQ ID NO: 312 - RNA, 16 nt, synthetic construct
  source 1..16: mol_type = other RNA
gtgtcatccattcatc

SEQ ID NO: 313 - RNA, 16 nt, synthetic construct
  source 1..16: mol_type = other RNA
tgtcatccattcatcc

SEQ ID NO: 314 - RNA, 16 nt, synthetic construct
  source 1..16: mol_type = other RNA
gtcatccattcatcca

SEQ ID NO: 315 - RNA, 16 nt, synthetic construct
  source 1..16: mol_type = other RNA
tcatccattcatccat

SEQ ID NO: 316 - RNA, 16 nt, synthetic construct
  source 1..16: mol_type = other RNA
catccattcatccatc

SEQ ID NO: 317 - RNA, 16 nt, synthetic construct
  source 1..16: mol_type = other RNA
atccattcatccatca

SEQ ID NO: 318 - RNA, 16 nt, synthetic construct
  source 1..16: mol_type = other RNA
atccattcatccatca

SEQ ID NO: 319 - RNA, 16 nt, synthetic construct
  source 1..16: mol_type = other RNA
ccattcatccatcatt

SEQ ID NO: 320 - RNA, 16 nt, synthetic construct
  source 1..16: mol_type = other RNA
cattcatccatcattc

SEQ ID NO: 321 - RNA, 16 nt, synthetic construct
  source 1..16: mol_type = other RNA
cattcatccatcattc

SEQ ID NO: 322 - RNA, 16 nt, synthetic construct
  source 1..16: mol_type = other RNA
ttcatccatcattcat

SEQ ID NO: 323 - RNA, 16 nt, synthetic construct
  source 1..16: mol_type = other RNA
tcatccatcattcatt

SEQ ID NO: 324 - RNA, 16 nt, synthetic construct
  source 1..16: mol_type = other RNA
catccatcattcattc

SEQ ID NO: 325 - RNA, 16 nt, synthetic construct
  source 1..16: mol_type = other RNA
atccatcattcattca

SEQ ID NO: 326 - RNA, 16 nt, synthetic construct
  source 1..16: mol_type = other RNA
tccatcattcattcat

SEQ ID NO: 327 - RNA, 16 nt, synthetic construct
  source 1..16: mol_type = other RNA
ccatcattcattcatc

SEQ ID NO: 328 - RNA, 16 nt, synthetic construct
  source 1..16: mol_type = other RNA
catcattcattcatcc

SEQ ID NO: 329 - RNA, 16 nt, synthetic construct
  source 1..16: mol_type = other RNA
atcattcattcatcca

SEQ ID NO: 330 - RNA, 16 nt, synthetic construct
  source 1..16: mol_type = other RNA
tcattcattcatccat

SEQ ID NO: 331 - RNA, 16 nt, synthetic construct
  source 1..16: mol_type = other RNA
cattcattcatccatt

SEQ ID NO: 332 - RNA, 16 nt, synthetic construct
  source 1..16: mol_type = other RNA
attcattcatccattc

SEQ ID NO: 333 - RNA, 16 nt, synthetic construct
  source 1..16: mol_type = other RNA
ttcattcatccattca

SEQ ID NO: 334 - RNA, 16 nt, synthetic construct
  source 1..16: mol_type = other RNA
tcattcatccattcat

SEQ ID NO: 335 - RNA, 16 nt, synthetic construct
  source 1..16: mol_type = other RNA
cattcatccattcatt

SEQ ID NO: 336 - RNA, 16 nt, synthetic construct
  source 1..16: mol_type = other RNA
attcatccattcattc

SEQ ID NO: 337 - RNA, 16 nt, synthetic construct
  source 1..16: mol_type = other RNA
ttcatccattcattca

SEQ ID NO: 338 - RNA, 16 nt, synthetic construct
  source 1..16: mol_type = other RNA
tcatccattcattcag

SEQ ID NO: 339 - RNA, 16 nt, synthetic construct
  source 1..16: mol_type = other RNA
catccattcattcagt

SEQ ID NO: 340 - RNA, 16 nt, synthetic construct
  source 1..16: mol_type = other RNA
atccattcattcagtg

SEQ ID NO: 341 - RNA, 16 nt, synthetic construct
  source 1..16: mol_type = other RNA
tccattcattcagtgc

SEQ ID NO: 342 - RNA, 16 nt, synthetic construct
  source 1..16: mol_type = other RNA
ccattcattcagtgct

SEQ ID NO: 343 - RNA, 16 nt, synthetic construct
  source 1..16: mol_type = other RNA
cattcattcagtgctt

SEQ ID NO: 344 - RNA, 16 nt, synthetic construct
  source 1..16: mol_type = other RNA
attcattcagtgcttg

SEQ ID NO: 345 - RNA, 16 nt, synthetic construct
  source 1..16: mol_type = other RNA
ttcattcagtgcttgt

SEQ ID NO: 346 - RNA, 16 nt, synthetic construct
  source 1..16: mol_type = other RNA
tcattcagtgcttgta

SEQ ID NO: 347 - RNA, 16 nt, synthetic construct
  source 1..16: mol_type = other RNA
cattcagtgcttgtat

SEQ ID NO: 348 - RNA, 16 nt, synthetic construct
  source 1..16: mol_type = other RNA
attcagtgcttgtatg

SEQ ID NO: 349 - RNA, 16 nt, synthetic construct
  source 1..16: mol_type = other RNA
ttcagtgcttgtatga

SEQ ID NO: 350 - RNA, 16 nt, synthetic construct
  source 1..16: mol_type = other RNA
tcagtgcttgtatgac

SEQ ID NO: 351 - RNA, 16 nt, synthetic construct
  source 1..16: mol_type = other RNA
cagtgcttgtatgact

SEQ ID NO: 352 - RNA, 16 nt, synthetic construct
  source 1..16: mol_type = other RNA
agtgcttgtatgactt

SEQ ID NO: 353 - RNA, 10 nt, synthetic construct
  source 1..10: mol_type = other RNA
atttgtctag

SEQ ID NO: 354 - RNA, 20 nt, synthetic construct
  source 1..20: mol_type = other RNA
aagtgttacagctcttttag

SEQ ID NO: 355 - RNA, 11 nt, synthetic construct
  source 1..11: mol_type = other RNA
aatttttggag

SEQ ID NO: 356 - RNA, 31 nt, synthetic construct
  source 1..31: mol_type = other RNA
caggttttctgacttcggtcggaaaacccct

SEQ ID NO: 357 - RNA, 14 nt, synthetic construct
  source 1..14: mol_type = other RNA
gtctacaatgaaag

SEQ ID NO: 358 - RNA, 66 nt, synthetic construct
  source 1..66: mol_type = other RNA
aactatccatccatgtactcacccaatttttggagcaggttttctgacttcggtcggaaaacccct

SEQ ID NO: 359 - RNA, 22 nt, synthetic construct
  source 1..22: mol_type = other RNA
ctatccatccatgtactcaccc

SEQ ID NO: 360 - RNA, 433 nt, synthetic construct
  source 1..433: mol_type = other RNA
gaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaactatccatccatgtactcacccaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtaagctt

SEQ ID NO: 361 - RNA, 65 nt, synthetic construct
  source 1..65: mol_type = other RNA
aatactcacccatctctccatccaatttttggagcaggttttctgacttcggtcggaaaacccct

SEQ ID NO: 362 - RNA, 21 nt, synthetic construct
  source 1..21: mol_type = other RNA
tactcacccatctctccatcc

SEQ ID NO: 363 - RNA, 63 nt, synthetic construct
  source 1..63: mol_type = other RNA
aatgtactcacccatctctccaatttttggagcaggttttctgacttcggtcggaaaacccct

SEQ ID NO: 364 - RNA, 20 nt, synthetic construct
  source 1..20: mol_type = other RNA
atgtactcacccatctctcc

SEQ ID NO: 365 - RNA, 66 nt, synthetic construct
  source 1..66: mol_type = other RNA
aacatccatgtactcacccatctcaatttttggagcaggttttctgacttcggtcggaaaacccct

SEQ ID NO: 366 - RNA, 22 nt, synthetic construct
  source 1..22: mol_type = other RNA
catccatgtactcacccatctc

SEQ ID NO: 367 - RNA, 68 nt, synthetic construct
  source 1..68: mol_type = other RNA
aatccatccatgtactcacccatctcaatttttggagcaggttttctgacttcggtcggaaaacccct

SEQ ID NO: 368 - RNA, 24 nt, synthetic construct
  source 1..24: mol_type = other RNA
tccatccatgtactcacccatctc

SEQ ID NO: 369 - RNA, 64 nt, synthetic construct
  source 1..64: mol_type = other RNA
aatccatgtactcacccatctcaatttttggagcaggttttctgacttcggtcggaaaacccct

SEQ ID NO: 370 - RNA, 20 nt, synthetic construct
  source 1..20: mol_type = other RNA
tccatgtactcacccatctc

SEQ ID NO: 371 - RNA, 64 nt, synthetic construct
  source 1..64: mol_type = other RNA
aaatctgttcaatcattcattcaatttttggagcaggttttctgacttcggtcggaaaacccct

SEQ ID NO: 372 - RNA, 20 nt, synthetic construct
  source 1..20: mol_type = other RNA
atctgttcaatcattcattc

SEQ ID NO: 373 - RNA, 63 nt, synthetic construct
  source 1..63: mol_type = other RNA
aactcattcatctgttcaatcaatttttggagcaggttttctgacttcggtcggaaaacccct

SEQ ID NO: 374 - RNA, 20 nt, synthetic construct
  source 1..20: mol_type = other RNA
actcattcatctgttcaatc

SEQ ID NO: 375 - RNA, 65 nt, synthetic construct
  source 1..65: mol_type = other RNA
aatctactcatcactcattcatcaatttttggagcaggttttctgacttcggtcggaaaacccct

SEQ ID NO: 376 - RNA, 21 nt, synthetic construct
  source 1..21: mol_type = other RNA
tctactcatcactcattcatc

SEQ ID NO: 377 - RNA, 64 nt, synthetic construct
  source 1..64: mol_type = other RNA
aacccaggcagctggaagagacaatttttggagcaggttttctgacttcggtcggaaaacccct

SEQ ID NO: 378 - RNA, 20 nt, synthetic construct
  source 1..20: mol_type = other RNA
cccaggcagctggaagagac

SEQ ID NO: 379 - RNA, 64 nt, synthetic construct
  source 1..64: mol_type = other RNA
aactgtgccacaagccgcattcaatttttggagcaggttttctgacttcggtcggaaaacccct

SEQ ID NO: 380 - RNA, 20 nt, synthetic construct
  source 1..20: mol_type = other RNA
ctgtgccacaagccgcattc

SEQ ID NO: 381 - RNA, 63 nt, synthetic construct
  source 1..63: mol_type = other RNA
aagtcaactgtgccacaagccgaatttttggagcaggttttctgacttcggtcggaaaacccc

SEQ ID NO: 382 - RNA, 20 nt, synthetic construct
  source 1..20: mol_type = other RNA
gtcaactgtgccacaagccg

SEQ ID NO: 383 - RNA, 64 nt, synthetic construct
  source 1..64: mol_type = other RNA
aatccttgtcaactgtgccacaaatttttggagcaggttttctgacttcggtcggaaaacccct

SEQ ID NO: 384 - RNA, 20 nt, synthetic construct
  source 1..20: mol_type = other RNA
tccttgtcaactgtgccaca

SEQ ID NO: 385 - RNA, 64 nt, synthetic construct
  source 1..64: mol_type = other RNA
aatatcatccttgtcaactgtgaatttttggagcaggttttctgacttcggtcggaaaacccct

SEQ ID NO: 386 - RNA, 20 nt, synthetic construct
  source 1..20: mol_type = other RNA
tatcatccttgtcaactgtg

SEQ ID NO: 387 - RNA, 68 nt, synthetic construct
  source 1..68: mol_type = other RNA
aatccttgtcaactgtgccacaagccaatttttggagcaggttttctgacttcggtcggaaaacccct

SEQ ID NO: 388 - RNA, 24 nt, synthetic construct
  source 1..24: mol_type = other RNA
tccttgtcaactgtgccacaagcc

SEQ ID NO: 389 - RNA, 24 nt, synthetic construct
  source 1..24: mol_type = other RNA
tccttgtcaactgtgccacaagcc

SEQ ID NO: 390 - RNA, 24 nt, synthetic construct
  source 1..24: mol_type = other RNA
agtcctgcaatatgaatataattt

SEQ ID NO: 391 - RNA, 68 nt, synthetic construct
  source 1..68: mol_type = other RNA
aatgccgagtcctgcaatatgaatataatttttggagcaggttttctgacttcggtcggaaaacccct

SEQ ID NO: 392 - RNA, 24 nt, synthetic construct
  source 1..24: mol_type = other RNA
tgccgagtcctgcaatatgaatat

SEQ ID NO: 393 - RNA, 68 nt, synthetic construct
  source 1..68: mol_type = other RNA
aatcttctgccgagtcctgcaatatgaatttttggagcaggttttctgacttcggtcggaaaacccct

SEQ ID NO: 394 - RNA, 24 nt, synthetic construct
  source 1..24: mol_type = other RNA
tcttctgccgagtcctgcaatatg

SEQ ID NO: 395 - RNA, 68 nt, synthetic construct
  source 1..68: mol_type = other RNA
aaggtcttctgccgagtcctgcaataaatttttggagcaggttttctgacttcggtcggaaaacccct

SEQ ID NO: 396 - RNA, 24 nt, synthetic construct
  source 1..24: mol_type = other RNA
ggtcttctgccgagtcctgcaata

SEQ ID NO: 397 - RNA, 68 nt, synthetic construct
  source 1..68: mol_type = other RNA
aacgaaggtcttctgccgagtcctgcaatttttggagcaggttttctgacttcggtcggaaaacccct

SEQ ID NO: 398 - RNA, 24 nt, synthetic construct
  source 1..24: mol_type = other RNA
cgaaggtcttctgccgagtcctgc

SEQ ID NO: 399 - RNA, 70 nt, synthetic construct
  source 1..70: mol_type = other RNA
aacttctgccgagtcctgcaatatgaataatttttggagcaggttttctgacttcggtcggaaaacccct

SEQ ID NO: 400 - RNA, 26 nt, synthetic construct
  source 1..26: mol_type = other RNA
cttctgccgagtcctgcaatatgaat

SEQ ID NO: 401 - RNA, 72 nt, synthetic construct
  source 1..72: mol_type = other RNA
aatccttgtcaactgtgccacaagccgcataatttttggagcaggttttctgacttcggtcggaaaacccct

SEQ ID NO: 402 - RNA, 28 nt, synthetic construct
  source 1..28: mol_type = other RNA
tccttgtcaactgtgccacaagccgcat

SEQ ID NO: 403 - RNA, 208 nt, synthetic construct
  source 1..208: mol_type = other RNA
ggctcgctacagaggcctttccgcaactatccatccatgtactcacccaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtaagcttgtattcagat

SEQ ID NO: 404 - RNA, 20 nt, synthetic construct
  source 1..20: mol_type = other RNA
ggggccactagggacaggat

SEQ ID NO: 405 - RNA, 19 nt, synthetic construct
  source 1..19: mol_type = other RNA
gagacttggtggtgcataa

SEQ ID NO: 406 - RNA, 19 nt, synthetic construct
  source 1..19: mol_type = other RNA
ggagaggacttgatcatta

SEQ ID NO: 407 - RNA, 22 nt, synthetic construct
  source 1..22: mol_type = other RNA
aaccgaacaggacctgaaagag

SEQ ID NO: 408 - RNA, 24 nt, synthetic construct
  source 1..24: mol_type = other RNA
cagtcacaccatcgtccatctatc

SEQ ID NO: 409 - RNA, 20 nt, synthetic construct
  source 1..20: mol_type = other RNA
tccatcatgaagtgtgacgt

SEQ ID NO: 410 - RNA, 20 nt, synthetic construct
  source 1..20: mol_type = other RNA
tcatccccaagccattcagg

SEQ ID NO: 411 - RNA, 20 nt, synthetic construct
  source 1..20: mol_type = other RNA
tcatccccaagccattcagg

SEQ ID NO: 412 - RNA, 20 nt, synthetic construct
  source 1..20: mol_type = other RNA
ccagaacatcatccctgcct

SEQ ID NO: 413 - RNA, 20 nt, synthetic construct
  source 1..20: mol_type = other RNA
ggtcaggtccaccactgaca

SEQ ID NO: 414 - RNA, 20 nt, synthetic construct
  source 1..20: mol_type = other RNA
acctgtctgcatgagaacct

SEQ ID NO: 415 - RNA, 21 nt, synthetic construct
  source 1..21: mol_type = other RNA
atggatggagagatggaacct

SEQ ID NO: 416 - RNA, 22 nt, synthetic construct
  source 1..22: mol_type = other RNA
gggctgtctcatcgtagtaaac

SEQ ID NO: 417 - RNA, 21 nt, synthetic construct
  source 1..21: mol_type = other RNA
gctaaaacagcaatggcctac

SEQ ID NO: 418 - RNA, 22 nt, synthetic construct
  source 1..22: mol_type = other RNA
ttgcttcacttccatatcatcg

SEQ ID NO: 419 - RNA, 21 nt, synthetic construct
  source 1..21: mol_type = other RNA
gctaaaacagcaatgggactc

SEQ ID NO: 420 - RNA, 20 nt, synthetic construct
  source 1..20: mol_type = other RNA
gcaggctgtctgtctctctc

SEQ ID NO: 421 - RNA, 19 nt, synthetic construct
  source 1..19: mol_type = other RNA
tcctcactctctgacgagg

SEQ ID NO: 422 - RNA, 17 nt, synthetic construct
  source 1..17: mol_type = other RNA
catggcggtcgacctag

SEQ ID NO: 423 - RNA, 20 nt, synthetic construct
  source 1..20: mol_type = other RNA
taccatccactcgacacacc

SEQ ID NO: 424 - RNA, 21 nt, synthetic construct
  source 1..21: mol_type = other RNA
agtcagtcaagctagcagagg

SEQ ID NO: 425 - RNA, 20 nt, synthetic construct
  source 1..20: mol_type = other RNA
gacatcaaatcccgcgtgaa

SEQ ID NO: 426 - RNA, 20 nt, synthetic construct
  source 1..20: mol_type = other RNA
cattgatgttggcgagcagg

SEQ ID NO: 427 - RNA, 19 nt, synthetic construct
  source 1..19: mol_type = other RNA
gctctctccgctgctgtag

SEQ ID NO: 428 - RNA, 20 nt, synthetic construct
  source 1..20: mol_type = other RNA
cgaggttccgggtaaaagca

SEQ ID NO: 429 - RNA, 21 nt, synthetic construct
  source 1..21: mol_type = other RNA
tactcctgcttgctgatccac

SEQ ID NO: 430 - RNA, 20 nt, synthetic construct
  source 1..20: mol_type = other RNA
ctgtctctctctctcgcaca

SEQ ID NO: 431 - RNA, 201 nt, Homo sapiens
  source 1..201: mol_type = mRNA
cccggcccaggatgacgtgcttattatttgacaggtgcatgtgacactgtgactccggctgtgacctgatggggcctcagggatgcgtctggctctggcaggatgtttgtgtgtcaccgcgatgttgtgtgggtgtgtctacctgtgccctgctctgagggattgagtgtgatatcgtgtgtttgtgctgcgctgtgatgg

SEQ ID NO: 432 - RNA, 65 nt, Homo sapiens
  source 1..65: mol_type = mRNA
gaccagaactattttttcccttacaccttgaccagcttgcatattggataccacatgattatcag

SEQ ID NO: 433 - RNA, 135 nt, Homo sapiens
  source 1..135: mol_type = mRNA
atcttttgtgtgtgtgtgtgtgtgtgtgtgtgtgtgtgtgtcacccaggctggagtgcagtggcatgatcacagctcactgcagcctcaacttcctgggctcaagtgatcctctcccgagtagctgggactacag

SEQ ID NO: 434 - RNA, 228 nt, Homo sapiens
  source 1..228: mol_type = mRNA
gtagcccctggctgtccttcagagggggcacaggtggagaaagaggcgcagtccctggctgtggtccctggagtgggtatacacgtgtgagtgtgtgcagatgtggaggtgagtaggcaagcgaagtgtatgtgtgtgcatggatgtattacaagtgtgtgcgtgtgggtgagtgtgcatgtctgggtgtgagtgtgcccgagactgcatgcatgtgtgtgtgtgagt

SEQ ID NO: 435 - RNA, 220 nt, Homo sapiens
  source 1..220: mol_type = mRNA
ctggactggggcaccactggctggcgtgagtgcacatgtgtgtgtttatgagtgtggctgtgaaatgtgagcacgtgcacctgtatgtatgtgtgtgggtgtttgcacgtgggggcgcgtgagcacatgaaatcagggtccatatgggtggggatgtgcatacatgtgcatgtgtattgtgtgaatgtatgagtgagcgtgtggaggtgtgtgcatgtgg

SEQ ID NO: 436 - RNA, 204 nt, Homo sapiens
  source 1..204: mol_type = mRNA
aagaaaagcgaggagcccttcaggttgtgcctatgaccctttgggtcgtccttttttgtctatttccctcccctccttcccaagcactgtatgtgtgtgtgtatgtgtgtgtgtgtgtgtacatgcacatgtgcgtgcatgatctgtgcctctgagctttggctcatgcagtctatttttctgaaaagcagtttgtgtgcatgc

SEQ ID NO: 437 - RNA, 41 nt, Homo sapiens
  source 1..41: mol_type = mRNA
gacgtgcttattatttgacaggtgcatgtgacactgtgact

SEQ ID NO: 438 - RNA, 59 nt, Homo sapiens
  source 1..59: mol_type = mRNA
acgtgcttattatttgacaggtgcatgtgacactgtgactccggctgtgacctgatggg

SEQ ID NO: 439 - RNA, 61 nt, Homo sapiens
  source 1..61: mol_type = mRNA
ggcctcagggatgcgtctggctctggcaggatgtttgtgtgtcaccgcgatgttgtgtggg

SEQ ID NO: 440 - RNA, 62 nt, Homo sapiens
  source 1..62: mol_type = mRNA
tgtgtcaccgcgatgttgtgtgggtgtgtctacctgtgccctgctctgagggattgagtgtg

SEQ ID NO: 441 - RNA, 42 nt, Homo sapiens
  source 1..42: mol_type = mRNA
ttttcccttacaccttgaccagcttgcatattggataccaca

SEQ ID NO: 442 - RNA, 32 nt, Homo sapiens
  source 1..32: mol_type = mRNA
acaccttgaccagcttgcatattggataccac

SEQ ID NO: 443 - RNA, 42 nt, Homo sapiens
  source 1..42: mol_type = other RNA
tgtgtgtgtgtgtgtcacccaggctggagtgcagtggcatga

SEQ ID NO: 444 - RNA, 96 nt, Homo sapiens
  source 1..96: mol_type = mRNA
atcttttgtgtgtgtgtgtgtgtgtgtgtgtgtgtgtgtgtcacccaggctggagtgcagtggcatgatcacagctcactgcagcctcaacttcct

SEQ ID NO: 445 - RNA, 32 nt, Homo sapiens
  source 1..32: mol_type = other RNA
agtgatcctctcccgagtagctgggactacag

SEQ ID NO: 446 - RNA, 40 nt, Homo sapiens
  source 1..40: mol_type = mRNA
gtgtgtgcagatgtggaggtgagtaggcaagcgaagtgta

SEQ ID NO: 447 - RNA, 61 nt, Homo sapiens
  source 1..61: mol_type = other RNA
aggcaagcgaagtgtatgtgtgtgcatggatgtattacaagtgtgtgcgtgtgggtgagtg

SEQ ID NO: 448 - RNA, 53 nt, Homo sapiens
  source 1..53: mol_type = mRNA
caagtgtgtgcgtgtgggtgagtgtgcatgtctgggtgtgagtgtgcccgaga

SEQ ID NO: 449 - RNA, 37 nt, Homo sapiens
  source 1..37: mol_type = other RNA
agtgtgcccgagactgcatgcatgtgtgtgtgtgagt

SEQ ID NO: 450 - RNA, 40 nt, Homo sapiens
  source 1..40: mol_type = mRNA
ggggcaccactggctggcgtgagtgcacatgtgtgtgttt

SEQ ID NO: 451 - RNA, 59 nt, Homo sapiens
  source 1..59: mol_type = mRNA
acatgtgcatgtgtattgtgtgaatgtatgagtgagcgtgtggaggtgtgtgcatgtgg

SEQ ID NO: 452 - RNA, 45 nt, Homo sapiens
  source 1..45: mol_type = mRNA
aagaaaagcgaggagcccttcaggttgtgcctatgaccctttggg

SEQ ID NO: 453 - RNA, 92 nt, Homo sapiens
  source 1..92: mol_type = mRNA
tcccaagcactgtatgtgtgtgtgtatgtgtgtgtgtgtgtgtacatgcacatgtgcgtgcatgatctgtgcctctgagctttggctcatgc

SEQ ID NO: 454 - RNA, 92 nt, Homo sapiens; identical to SEQ ID NO: 453
  source 1..92: mol_type = mRNA

SEQ ID NO: 455 - RNA, 92 nt, synthetic construct; identical to SEQ ID NO: 453
  source 1..92: mol_type = other RNA

SEQ ID NO: 456 - RNA, 20 nt, synthetic construct
  source 1..20: mol_type = other RNA
tatgttagggacttagggag

SEQ ID NO: 457 - RNA, 21 nt, synthetic construct
  source 1..21: mol_type = other RNA
tgtggggaccttgtggggacc

SEQ ID NO: 458 - RNA, 17 nt, synthetic construct
  source 1..17: mol_type = other RNA
tgtggggaccaggggga

SEQ ID NO: 459 - RNA, 1897 nt, synthetic construct
  source 1..1897: mol_type = other RNA
gaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgtccatgtactcacccatctcaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgtcgacgaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgcacagagagccaaattcttaaatttttggagcaggttttctgacttcggtcggaaaacccctcccaagttaactggtctacaatgaaagcaaaacagttctcttccccgctccccggtgtgtgagaggggctttgatccttctctggtttcctaggaagctttacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcaggatccgaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgcccgtatccggtactatatgaatttttggagcaggttttctgacttcggtcggaaaacccctcccaagttaactggtctacaatgaaagcaaaacagttctcttccccgctccccggtgtgtgagaggggctttgatccttctctggtttcctaggaagatctatggcagcggccgcatgatatctagactggcctgggcctcatgggccttccgctc

SEQ ID NO: 460 - RNA, 439 nt, synthetic construct
  source 1..439: mol_type = other RNA
gaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgtccatgtactcacccatctcaatttttggagcaggttttctgacttcggtcggaaaacccctcccaagttaactggtctacaatgaaagcaaaacagttctcttccccgctccccggtgtgtgagaggggctttgatccttctctggtttcctaggaa

SEQ ID NO: 461 - RNA, 20 nt, synthetic construct
  source 1..20: mol_type = other RNA
tccatgtactcacccatctc

SEQ ID NO: 462 - RNA, 439 nt, synthetic construct
  source 1..439: mol_type = other RNA
gaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgcacagagagccaaattcttaaatttttggagcaggttttctgacttcggtcggaaaacccctcccaagttaactggtctacaatgaaagcaaaacagttctcttccccgctccccggtgtgtgagaggggctttgatccttctctggtttcctaggaa

SEQ ID NO: 463 - RNA, 20 nt, synthetic construct
  source 1..20: mol_type = other RNA
cacagagagccaaattctta

SEQ ID NO: 464 - RNA, 499 nt, synthetic construct
  source 1..499: mol_type = other RNA
gaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgcccgtatccggtactatatgaatttttggagcaggttttctgacttcggtcggaaaacccctcccaagttaactggtctacaatgaaagcaaaacagttctcttccccgctccccggtgtgtgagaggggctttgatccttctctggtttcctaggaagatctatggcagcggccgcatgatatctagactggcctgggcctcatgggccttccgctc

SEQ ID NO: 465 - RNA, 20 nt, synthetic construct
  source 1..20: mol_type = other RNA
cccgtatccggtactatatg

SEQ ID NO: 466 - RNA, 20 nt, synthetic construct
  source 1..20: mol_type = other RNA
tatgatagggacttagggtg

SEQ ID NO: 467 - RNA, 40 nt, synthetic construct
  source 1..40: mol_type = other RNA
agatccagtttatcgataccaacataggagctgtgattgg

SEQ ID NO: 468 - RNA, 38 nt, synthetic construct
  source 1..38: mol_type = other RNA
atgaattactcatcggcgagaaaggaagggaagaaagc

SEQ ID NO: 469 - RNA, 19 nt, synthetic construct
  source 1..19: mol_type = other RNA
tgggaccgctttacgcttc

SEQ ID NO: 470 - RNA, 21 nt, synthetic construct
  source 1..21: mol_type = other RNA
gagactggctgactcgttgac

SEQ ID NO: 471 - RNA, 19 nt, synthetic construct
  source 1..19: mol_type = other RNA
ctctgggactggagcaaac

SEQ ID NO: 472 - RNA, 19 nt, synthetic construct
  source 1..19: mol_type = other RNA
catcccgtatccggtaagg

SEQ ID NO: 473 - RNA, 1211 nt, synthetic construct
  source 1..1211: mol_type = other RNA
tatttccggtgaattcgccgccaccatggtttccaagggcgaagaggacaacatggccatcatcaaagaattcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagaccatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaaaggctctggcgaaggccgtggcagcctgcttacatgcggagatgtggaagagaaccccggacctatggccaagcctctgagccaagaggaaagcaccctgatcgaaagagccaccgccacaatcaacagcatccccatcagcgaggattacagcgtggcctctgctgccctcagctccgatggcagaatcttcacaggcgtgaacgtgtaccacttcaccggcggaccttgtgccgaactggtggttcttggaacagctgccgctgccgcagccggcaatctgacatgtattgtggccatcggcaacgagaaccggggcatccttagtccttgcggcagatgcagacaggtgctgctggatctgcaccctggcatcaaggccattgtgaaggactctgacggccagcctacagccgtgggaattagagagctgctgcctagcggctatgtgtgggagggatgaacgcgtctggaacaat

SEQ ID NO: 474 - RNA, 43 nt, synthetic construct
  source 1..43: mol_type = other RNA
taatttctactcttgtagatggaaaagtaaaagatgtctgaat

SEQ ID NO: 475 - RNA, 20 nt, synthetic construct
  source 1..20: mol_type = other RNA
ctggcgaggcatcacatttt

SEQ ID NO: 476 - RNA, 21 nt, synthetic construct
  source 1..21: mol_type = other RNA
cgttctcatcttcggttaccc

SEQ ID NO: 477 - RNA, 20 nt, synthetic construct
  source 1..20: mol_type = other RNA
aacgacattgccctgaagac

SEQ ID NO: 478 - RNA, 18 nt, synthetic construct
  source 1..18: mol_type = other RNA
ccagtacggctcccatct

(19) United States
(12) Patent Application Publication
(10) Pub. No.: US 2025/0361506 A1
(43) Pub. Date: Nov. 27, 2025

(54) MODIFIED U7 SNRNA CONSTRUCT

(71) Applicants: UCL BUSINESS LTD, London, Greater London (GB); KING'S COLLEGE LONDON, London, Greater London (GB)

(72) Inventors: Pietro FRATTA, London (GB); Marc-David RUEPP, London (GB); Christopher SHAW, London (GB)

(73) Assignees: UCL BUSINESS LTD, London, Greater London (GB); KING'S COLLEGE LONDON, London, Greater London (GB)

(21) Appl. No.: 18/872,509

(22) PCT Filed: Jun. 7, 2023
(86) PCT No.: PCT/EP2023/065287

§ 371 Date: Dec. 6, 2024

(30) Foreign Application Priority Data
Jun. 8, 2022 (GB) 2208384.4

(51) Int. Cl. C12N 15/113

(52) U.S. Cl. CPC C12N 15/113; C12N 2310/14; C12N 2320/33

(57) ABSTRACT

A modified U7 snRNA construct comprising at least one antisense sequence having between 16 to 30 nucleotides which are at least 90% complementary to a splicing element of a TDP-43 regulated cryptic exon sequence or flanking regions thereof and wherein the U7 snRNA construct is capable of modulating splicing of the TDP-43 regulated cryptic exon in a cell. The splicing element may be selected from a splice site, a TDP-43 binding region, or an exonic splice enhancer. Vectors and pharmaceutical compositions comprising the construct are also described, as well as constructs for use in the treatment of diseases associated with TDP-43 dysfunction. Example TDP-43 regulated cryptic exon sequences include TDP-43 regulated cryptic exons in UNC13A, STMN2 and INSR genes.

### BACKGROUND

Loss of nuclear TDP-43 is observed in a number of diseases or disorders including >95% of all Amyotrophic Lateral Sclerosis (ALS) and tau-negative Frontotemporal Dementia (FTD) cases. This results in the inclusion of cryptic exons (CE) with subsequent functional loss of important disease-modifying genes, due to the absence of TDP-43 repression of these cryptic exons. TDP-43 regulated cryptic exons in both STMN2 and UNC13A have been mechanistically linked to ALS and FTD: STMN2 and UNC13A encode an axonal and synaptic protein, respectively and are crucial for normal neuronal function. In both cases, loss of nuclear TDP-43 results in the incorporation of a CE during splicing resulting in the depletion of the full-length mRNA and reduction of functional protein expression. Loss of nuclear TDP-43 also results in aberrant RNA processing, with STMN2 being the most significantly affected. Its depletion results in impaired axonal regeneration, which is alleviated when STMN2 levels are restored. For UNC13A human genetic evidence supports its impact in disease aetiology: Intronic SNPs in UNC13A are the second strongest risk factor for sporadic ALS, are associated with reduced patient survival, and shown to directly enhance cryptic exon inclusion.

TDP-43 regulated cryptic exons (CEs) are also known to affect numerous other transcripts which have crucial neuronal functions. One such example is in the ELAVL3 gene which encodes for a neuronal-specific RNA binding protein. The ELAVL3 CE leads to protein loss, which has been documented in ALS post mortem neurons, and leads to alterations in neurite maturation, maintenance. Similarly, TDP-43 loss induces a CE and consequent loss of another neuronal-specific RNA binding protein, CELF5, loss of which is known to cause motor neuron degeneration in model systems. CEs also appears in the INSR transcript leading to its reduction, with insulin signalling having emerged as an important pathway for neuronal health and maintenance.

There is therefore a need to further understand the role of TDP-43 depletion in disease, and to generate new therapeutic approaches for alleviating diseases associated with TDP-pathology, including but not limited to neurodegeneration, particularly in ALS/FTD.

### SUMMARY OF INVENTION

According to a first aspect of the present invention, is provided a modified U7 snRNA construct comprising at least one antisense sequence having between 16 to 30 nucleotides which are at least 90% complementary to a splicing element of a TDP-43 regulated cryptic exon sequence and/or flanking regions thereof, and wherein the construct is capable of modulating splicing of the TDP-43 regulated cryptic exon in a cell (i.e., the construct is capable of at least partially preventing inclusion of the TDP-43 regulated cryptic exon in a mature RNA product). Flanking regions described herein may be defined as 25 nucleotides upstream and downstream of the splicing element, or in some 20 nucleotides upstream and downstream of the splicing element. In other embodiments, the splicing element or flanking regions thereof may be defined by a particular sequence for a specific TDP-43 regulated cryptic exon (e.g., SEQ ID NO: 11-40 or SEQ ID NO 437-454).

The antisense sequence directs the construct to splicing elements of the TDP-43 regulated cryptic exon and critically blocks the splicing machinery from splicing the cryptic exon (i.e., in the pre-mRNA). These components act to repress splicing of the cryptic exon, even in the absence of TDP-43 binding, or in cells depleted of TDP-43, such that the cryptic exon is at least partially excluded in the mature RNA of the cell transcript. This restores the functionality of genes containing TDP-43 regulated cryptic exons, e.g., in cells depleted of TDP-43. The constructs herein can therefore be used to further probe, understand, or treat diseases or disorders characterised by TDP-43 dysfunction or pathology

In some embodiments, the splicing element is a splice site. Constructs comprising antisense sequences that target the splice sites means that the splice sites are masked and less available for splicing by the above-mentioned splicing machinery within the cell.

In some embodiments, the splicing element is a TDP-43 binding region. Since TDP-43 has as repressive role in healthy cells, and blocks splicing machinery from recognising the cryptic exon, constructs comprising antisense sequences that target the TDP-43 binding region serve to provide a steric block within this region, thereby fulfilling the role of TDP-43 in cells that are depleted of TDP-43.

In some embodiments, the splicing element is an exonic splice enhancer, e.g., as defined by ESE finder 3.0, or as defined as a binding site or motif for an SR protein (such as SRSF1, SRSF2, SRSF5 or SRSF6). Since ESEs are motifs within the cryptic exon sequence that promote or enhance splicing, blocking these motifs blocks cryptic splicing of the cryptic exon sequence.

According to a second aspect of the present invention, is provided a vector that comprises or encodes for the modified U7 snRNA construct of the first aspect. In some embodiments, the vector is a viral vector.

According to a third aspect of the present invention, is provided a pharmaceutical composition comprising one or more of the constructs according to the first aspect, or one or more of the vectors according to the second aspect.

According to a fourth aspect of the present invention, is provided the construct of the first aspect, the vector of the second aspect or the pharmaceutical composition of the third aspect for use in therapy. Also disclosed herein is the construct of the first aspect, the vector of the second aspect or the pharmaceutical composition of the third aspect for use as a medicament, for use in the manufacture of a medicament, or for use in a method of treatment (e.g., for a neurodegenerative or muscular disease or disorder).

According to a fifth aspect of the present invention, is provided the construct of the first aspect, the vector of the second aspect, or the pharmaceutical composition of the third aspect, for use in the treatment of a disease characterised by TDP-43 dysfunction (or TDP-43 pathology). In some embodiments, the disease is a neurodegenerative or muscular disease, and optionally wherein the disease is Amyotrophic lateral sclerosis (ALS) and frontotemporal dementia (FTD), Inclusion body myositis or myopathy (IBM), Alzheimer's disease, FOSMNN (Facial onset sensory and motor neuronopathy), Perry Syndrome, Limbic-Predominant Age-Related TDP-43 Encephalopathy (LATE) or a combination thereof.

According to a sixth aspect of the present invention, is a method of correcting splicing of a TDP-43 regulated cryptic exon in a cell, the method comprising delivering to a cell the construct of the first aspect, the vector of the second aspect, or the pharmaceutical composition of the third aspect, wherein the method comprises contacting the construct with a cell, and wherein the construct modulates splicing of the TDP-43 regulated cryptic exon in the cell.

According to a seventh aspect of the present invention, is a combined vector comprising two or more of the constructs described herein. (i.e., in tandem, or one downstream of another, such that the construct comprises more than one antisense sequence as defined herein). In preferred embodiments, the two or more modified U7 snRNA constructs comprise different antisense sequences that are capable of binding to (i.e., at least 90%, or at least 95%, or 100% complementary to) different TDP-43 regulated cryptic exon sequences as described herein (i.e., which are present in the pre-mRNA). For example, the combined vector may comprise a first construct comprising an antisense sequence which is at least 90% complementary to (or at least 95%, or 100% complementary to) a first TDP-43 regulated cryptic exon, and a second construct comprising an antisense sequence which is at least 90% complementary to (or at least 95%, or at least 100% complementary to) a second TDP-43 regulated cryptic exon, wherein the second TDP-43 regulated cryptic exon is different from the first. In some embodiments, the combined vector may comprise three or more constructs as defined herein. The TDP-43 regulated cryptic exon may be any TDP-43 regulated cryptic exon as described herein. In some embodiments, the antisense sequence(s) are sequence(s) which are at least 90% complementary (or at least 95%, or 100% complementary) to SEQ ID NO: 1, 2, 3, 4, 7, 9 or 431-436). In some embodiments, at least one of the antisense sequences, or each antisense sequences, is complementary to a TDP-43 binding region of the TDP-43 regulated cryptic exon, preferably wherein at least one of the antisense sequences, or each antisense sequence, is complementary (i.e., 90%, 95% or 100% complementary) to SEQ ID NO: 12, 23-26 or 32. In some embodiments, the combined vector comprises a construct as defined herein comprising an antisense sequence which is at least 90% (or at least 95%, or 100%) complementary to a UNC13A TDP-43 regulated cryptic exon or flanking region thereof and a construct as defined herein comprising an antisense sequence which is at least 90% (or at least 95%, or 100%) complementary to a STMN2 TDP-43 regulated cryptic exon or flanking region thereof. In some embodiments, the combined vector comprises a construct as defined herein comprising an antisense sequence which is at least 90% (or at least 95%, or 100%) complementary to UNC13A TDP-43 regulated cryptic exon or flanking region thereof and a construct as defined herein comprising an antisense sequence which is at least 90% (or at least 95%, or 100%) complementary to a INSR TDP-43 regulated cryptic exon or flanking region thereof. In some embodiments, the combined vector comprises a construct as defined herein comprising an antisense sequence which is at least 90% (or at least 95%, or 100%) complementary to a STMN2 TDP-43 regulated cryptic exon or flanking region thereof and a construct as defined herein comprising an antisense sequence which is at least 90% (or at least 95%, or 100%) complementary to a INSR TDP-43 regulated cryptic exon or flanking region thereof. In some embodiments, the combined vector comprises a construct comprising an antisense sequence which is at least 90% (or at least 95%, or 100%) complementary to a UNC13A TDP-43 regulated cryptic exon or flanking region thereof, a construct comprising an antisense sequence which is at least 90% (or at least 95%, or 100%) complementary to a STMN2 TDP-43 regulated cryptic exon or flanking region thereof, and a construct comprising an antisense sequence which is at least 90% (or at least 95%, or 100%) complementary to a INSR TDP-43 regulated cryptic exon or flanking region thereof. In some embodiments, at least one of the two or more constructs in the combined vector further comprises a binding domain sequence for a hnRNP protein, for example, for a hnRNP A or hnRNP H protein. In some embodiments, the combined vector comprises more than one binding domain sequence for a hnRNP protein. In some embodiments, each construct (i.e., constituent construct) comprises a binding domain sequence for a hnRNP proteins such that the combined vector comprises the same number of binding domain sequences for hnRNP proteins as there are antisense sequences.

In some embodiments, the combined vector comprises two or more promoter sequences, wherein the two or more promoter sequences are upstream of each construct. The promoters may be any promoter sequence used in the art. In some embodiments, each of the two or more promoter sequences are the same or different. In some embodiments, the combined vector comprises two or more 3′ box sequences, wherein the two or more 3′ box sequences are downstream of each construct. The 3′ box sequences may be the same or different and may be any 3′ box sequence used in the art.

In some embodiments, the combined vector comprises two or more U7 cassettes, wherein each cassette comprises a promoter, a modified U7 snRNA construct (e.g., as defined herein) and a 3′ box sequence, wherein the promoter is upstream of the modified U7 snRNA construct and the 3′ box sequence is downstream of the modified U7 snRNA construct. In some embodiments, the combined vector comprises a stuffer sequence between each of the two or more U7 cassettes. The stuffer sequences serve to space out the at least two promoters. The stuffer sequence may be any suitable stuffer sequence used in the art.

The present inventors have developed tools that can target TDP-43 regulated cryptic exons and modulate their aberrant splicing upon depletion of TDP-43, to prevent inclusion of the cryptic exon in mature RNA, thereby preventing loss of the translated protein such that fully functional protein is produced. There are a number of TDP-43 regulated cryptic exons that are aberrantly spliced upon depletion of TDP-43 in the nucleus. TDP-43 depletion is associated with a number of diseases including neurodegenerative and muscular diseases. TDP-43 regulated cryptic exons are characterised by a TDP-43 binding region either within the cryptic exon or in close proximity to the cryptic exon (i.e., in the flanking regions of the cryptic exon), said TDP-43 binding region typically being UG rich. During normal splicing, TDP-43 (i.e., a transcriptional repressor protein) binds to the binding domain and represses splicing of the cryptic exon; this has the effect that the cryptic exon is not included in the mature mRNA of the transcript and a functional protein is produced. However, depletion of TDP-43 from the nucleus of cells means that the cryptic exon sequence is aberrantly spliced; this has the effect that the cryptic exon is included in the mature mRNA of the transcript meaning functional protein is not produced.

The constructs, vectors and pharmaceutical compositions disclosed herein can be used to sterically mask crucial elements (i.e., splice elements) of cryptic exon splicing in the absence of TDP-43, to at least partially correct and prevent cryptic splicing. Crucially, the U7 constructs disclosed herein comprise an antisense sequence that guides the U7 snRNP to bind to a splicing element within the target cryptic exon, which therefore represses splicing of the cryptic exon and prevents its inclusion into the mature mRNA of the cell transcript. This therefore restores, at least partially, “normal” protein production which occurs in healthy cells without TDP-43 depletion.

Example constructs described herein are found to effectively correct splicing for different antisense sequences that target different splicing elements of the TDP-43 regulated cryptic exons (i.e., present in pre-mRNA). For example, in some examples, the antisense sequence binds to a TDP-43 binding region of a TDP-43 regulated cryptic exon, while correcting splicing. In alternative examples, the antisense sequence binds to a splice site of the TDP-43 regulated cryptic exon while correcting splicing. Finally, it is also demonstrated that correct splicing is restored when the antisense sequence binds to an exonic splice enhancer (i.e., SR protein binding sites as identified by ESE finder 3.0) located within the TDP-43 regulated cryptic exon. This demonstrates that the constructs can target a wide range of different target sequences within the TDP-43 regulated cryptic exon and flanking regions thereof, while still being effective. Further the present application provides evidence that this approach can be used to restore splicing for a number of different TDP-43 regulated cryptic exons located in different genes.

To the present inventor's knowledge, there has been no similar approach targeting TDP-43 regulated cryptic exons in the prior art. While other U7 modified constructs have been previously developed for use in gene therapy, other prior art constructs have completely different targets and instead sought to target standard constitutive exons rather than cryptic exons. The difference is that cryptic exons are non-conserved intronic sequences that are erroneously included in mature RNA, as opposed to a typical constitutive exon which is supposed to be included in mature RNA. Previous U7 modified constructs therefore had a different aim, or different approach (to promote exon inclusion and reduce gene expression of said gene), as opposed to the present invention which aims to rescue splicing and restore expression in the absence of TDP-43. The prior art constructs also have completely different targets, different modes of action and completely different uses. No prior art constructs have been used to correct or at least partially rescue “normal” splicing of TDP-43 regulated cryptic exons in order to restore the correct splicing of genes depleted in the cell when TDP-43 is depleted. As such, the constructs can be used to further understand, or treat, diseases associated with depleted TDP-43 pathology.

A major advantage of this approach is that snRNPs naturally reside in the nucleus where cryptic exon splicing needs to be repressed. This results in localisation of the antisense containing U7 snRNA in the cellular compartment where splicing needs to be corrected. The use of antisense sequences in snRNPs also provides enhanced stability of the resultant RNA-protein complexes with the pre-mRNA (i.e., which contains the cryptic exon).

Another advantage is that modified snRNPs can be packaged into vectors, such as viral vectors, that enable long lasting manufacture of the gene therapy following a single injection. This allows cells to produce their own therapeutic molecules as a single dose gene therapy, and is therefore improved as compared to ASO approaches. These constructs also provide a more stable therapeutic approach as compared to ASO targeting which are more sensitive to degradation. The small size of the U7 expression cassette also allows their delivery in combination with other antisense or supplemental gene constructs in a single viral vector or ITR cassette. Additionally, it is hypothesised that the larger size of the modified U7 snRNA construct as compared to an ASO approach could, in some instances, be more effective at correcting splicing due to steric effects since the constructs may also provide a more effective steric block which contributes to the repression of the cryptic splicing event. Further, ASO approaches vary significantly from U7 modified approaches in that ASOs typically comprise chemical modifications to increase binding affinity. Such chemical modifications are incompatible with a U7 modified construct approach. It is well understood that a simple copy and paste of known antisense sequences into a U7 modified construct would not be reasonably expected to result in similar efficient binding to a target. In fact, previous literature has shown that targeting the same sequence element with a U7 smOPT can result in an opposite outcome as compared to an ASO approach.

Since aspects of the invention are demonstrated to at least partially correct the splicing of TDP-43 regulated cryptic exons, aspects of the present invention can therefore be used to probe TDP-43 pathology and/or the role of TDP-43 pathology in disease. For example, as TDP-43 clearance is happening in >95% of ALS cases this approach is applicable and beneficial for the vast majority of ALS patients.

In some embodiments, constructs of the invention can be used to correct splicing of the TDP-43 regulated UNC13A cryptic exon. This cryptic exon is found to cause UNC13A downregulation at the transcript and protein level and is detected specifically in patient post-mortem brain regions affected by TDP-43 proteinopathy or dysfunction, including both ALS and FTD. Further, this cryptic exon is also found to overlap with the disease-associated variant rs12973192 previously identified in multiple genome-wide association studies linked to ALS/FTLD risk, as well as disease aggressiveness. The UNC13A cryptic exon is therefore associated with TDP pathology, and disease aggressiveness. Correcting splicing of the UNC13A gene can therefore be used to further understand and/or treat diseases associated with ALS and FTD, and SNPs (e.g., rs12973192) in the UNC13A gene.

In some embodiments, constructs of the invention can be used to correct splicing of the TDP-43 regulated STMN2 cryptic exon 2a. This is important considering loss of nuclear TDP-43 results in the incorporation of this cryptic exon during splicing resulting in the depletion of the full-length mRNA and reduction of functional protein expression. This effect is most pronounced for STMN2, where aberrant RNA processing results in impaired axonal regeneration. Correcting splicing of the STMN2 gene can therefore be used to further understand and/or treat diseases associated with TDP-43.

Embodiments of the present invention are also used to correct splicing of the TDP-43 regulated INSR cryptic exon (between INSR exons 6 and 7). The INSR CE leads to loss of the protein, which normally acts as a receptor for insulin. Insulin signalling plays an important role in neuronal maintenance, and restoration of INSR levels would contribute to an amelioration of neuronal homeostasis.

The constructs, vectors and compositions disclosed herein in some embodiments can also be used to correct splicing of other TDP-43 regulated cryptic exons, including the ELAVL3 cryptic exon, the G3BP1 cryptic exon, the AARS1 cryptic exon, the CELF5 cryptic exon, the CAMK2B cryptic exon, the UNC13B cryptic exon or the CELF5 cryptic exon. In particular, the ELAVL3 CE leads to alterations in neurite maturation and is implicated in ALS, while the CELF5 CE leads to motor neuron degeneration in model systems. Preventing cryptic splicing and restoration of these proteins is considered to be therapeutically beneficial.

The design of constructs described herein may be preferred over other conceivable designs that instead could rely on the recruitment of other endogenous splicing repressor proteins to the TDP-43 regulated cryptic exon, i.e., to fulfil the role of TDP-43. The recruitment of other splicing repressor proteins to the TDP-43 regulated cryptic exons may be disadvantageous since there is no risk of sequestering these proteins to the U7 constructs, which could affect the splicing of other mRNAs. A further advantage of the constructs of the present invention is that the 5′-end of the modified snRNA is kept relatively short, (i.e., by comprising only a relatively short antisense sequence of between 16 to 30 nucleotides that targets the TDP-43 regulated cryptic exon). This may be advantageous because it is hypothesised that longer or extended 5′ ends might affect snRNP assembly and hence reduce expression of the constructs.

Also described herein is a modified U7 snRNA construct comprising at least one antisense sequence having between 16 to 30 nucleotides which are at least 90% complementary to a splicing element of a TDP-43 regulated cryptic exon sequence, or flanking regions thereof, wherein the flanking regions are defined as the 25 nucleotides upstream and downstream of the splicing element. In some embodiments, the flanking regions are defined as 20 nucleotides upstream and downstream of the splicing element

Also described herein is a modified U7 snRNA construct comprising at least one antisense sequence having between 16 to 30 nucleotides which are at least 90% complementary to a splice site of a TDP-43 regulated cryptic exon sequence and flanking regions thereof, wherein the flanking regions are defined as the 20 nucleotides upstream and downstream of the splicing element.

Also described herein is a modified U7 snRNA construct comprising at least one antisense sequence having between 16 to 30 nucleotides which are at least 90% complementary to a exonic splice enhancer of a TDP-43 regulated cryptic exon sequence and flanking regions thereof, wherein the flanking regions are defined as the 20 nucleotides upstream and downstream of the splicing element, wherein the exonic splice enhancer is one defined by ESE finder 3.0.

Also described herein is a modified U7 snRNA construct comprising at least one antisense sequence having between 16 to 30 nucleotides which are at least 90% complementary to a TDP-43 region site of a TDP-43 regulated cryptic exon sequence and flanking regions thereof. It is particularly surprising that single targets directed against the TDP-43 binding region suppress cryptic exon splicing. This suggests that a snRNP moiety might be able substitute for the repressive function of TDP-43 (e.g., as shown for Examples 1G, 1H, 1I in UNC13A).

Also described herein is a modified U7 snRNA construct comprising (i) an antisense sequence having between 16 to 30 nucleotides which are at least 90% complementary to a splicing element in the TDP-43 regulated UNC13A cryptic exon sequence and flanking regions thereof, preferably wherein the antisense sequence is at least 90% complementary to SEQ ID NO: 1 or 2. In some embodiments, the antisense sequence is at least 90% complementary with SEQ ID NO: 3 or 4. In some embodiments, the splicing element is a 5′ splice site. In some embodiments, the splicing element is a 3′ splice site. In some embodiments, the splicing element is an ESE. In some embodiments, the splicing element is a TDP-43 binding region.

Also described herein is a modified U7 snRNA construct comprising (i) an antisense sequence having between 16 to 30 nucleotides which are at least 90% complementary to a splicing element in the TDP-43 regulated STMN2 cryptic exon sequence and flanking regions thereof, preferably wherein the antisense sequence is at least 90% complementary to SEQ ID NO: 7. In some embodiments, the splicing element is a 3′ splice site. In some embodiments, the splicing element is an ESE. In some embodiments, the splicing element is a TDP-43 binding region.

Also described herein is a modified U7 snRNA construct comprising (i) an antisense sequence having between 16 to 30 nucleotides which are at least 90% complementary to a splicing element in the TDP-43 regulated INSR cryptic exon sequence and flanking regions thereof, preferably wherein the antisense sequence is at least 90% complementary to SEQ ID NO: 9. In some embodiments, the splicing element is a 3′ splice site. In some embodiments, the splicing element is an ESE. In some embodiments, the splicing element is a TDP-43 binding region.

Also disclosed herein is a system comprising a construct, vector, or pharmaceutical composition and a cell, wherein said cell comprises or expresses a hnRNP protein. The cell may be as elsewhere defined herein.

For any sequence disclosed herein, the complementary sequence or reverse complement of the sequence is also disclosed. Also disclosed herein is a vector or construct with a complementary sequence to that described herein which may be used to encode for the constructs described herein.

### BRIEF DESCRIPTION OF FIGURES

FIG. 1A shows a schematic of TDP-43 regulated splicing in healthy cells (top) and diseased cells depleted of TDP-43 (bottom). In healthy cells, TDP-43 binds to a TDP-43 binding region within or in close proximity to the TDP-43 regulated cryptic exon (i.e., in pre-mRNA), and represses splicing of the cryptic exon such that no cryptic exon is included in mature mRNA of the cell transcript. In diseased cells, depletion of TDP-43 leads to cryptic splicing such that a cryptic exon is present in the mature RNA; FIG. 1B) shows a schematic of how the modified U7 snRNA construct of the invention can restore correct splicing in TDP-43 depleted cells. The modified U7 snRNA construct binds to splicing elements of the TDP-43 regulated cryptic exon and represses splicing by sterically blocking splicing factors from splicing the cryptic exon. This prevents, at least partially, inclusion of the cryptic exon in mature mRNA of the cell transcript.

FIG. 2 shows RT-PCR product of the UNC13A mature RNA in TDP-43 knockdown SK-N-DZs cells transfected with the UNC13A minigene after treatment with an example U7 snRNA construct of the invention (i.e., corresponding to Example 1A). A band is observed corresponding to the correctly spliced product when treated with the construct of the invention, along with disappearance of bands corresponding to mature RNA products that contain both the long cryptic exon and the short cryptic exon.

FIG. 3 shows the % differential splicing of the correctly spliced mature RNA (far left bar), mature RNA comprising the short UNC13A cryptic exon (middle bar) and mature RNA comprising the long UNC13A cryptic exon (far right bar) in SK-N-DZs cells transfected with a UNC13A minigene with TDP-43 knockdown after treatment with an example modified U7 snRNA construct of the invention (i.e., corresponding to Example 1A). Cells treated with the construct of the invention showed 100% correct splicing.

FIG. 4 shows RT-PCR product of the UNC13A mature RNA in TDP-43 depleted SH-SY5Y cells after treatment with an example modified U7 snRNA construct of the invention (i.e., corresponding to Example 1A) compared with controls. A greater proportion of correctly spliced mature RNA product is observed upon treatment with the construct of the invention as compared with controls.

FIG. 5 shows the % differential splicing of the correctly spliced mature RNA (far left bar), mature RNA comprising the short UNC13A cryptic exon (middle bar) and mature RNA comprising the long UNC13A cryptic exon (far right bar) deriving from endogenous UNC13A in electroporated TDP-43 depleted SH-SY5Y cells after treatment with an example U7 smOPT construct of the invention (i.e., corresponding to Example 1A). A greater proportion of correctly spliced mature RNA product is observed upon treatment with the construct of the invention as compared with controls.

FIGS. 6A-6C show the ratio of cryptic exon splicing to correct splicing of the UNC13A minigene in the presence of different modified U7 snRNA constructs of the invention containing either A) an antisense sequence that targets the 5′-splice site, B) an antisense sequence that targets the TDP-43 binding region and C) an antisense sequence that targets the 3′-splice site.

FIG. 7 shows partial rescue of STMN2 cryptic splicing using the example modified U7 snRNA constructs of the invention (i.e., corresponding to Example 2K and 2J) in TDP-43 depleted electroporated SH-SY5Y cells.

FIG. 8 shows the differential splicing of the correctly spliced mature RNA (left bar) compared with mature RNA containing the STMN2 cryptic exon (right bar) using example modified U7 snRNA constructs of the invention (i.e., corresponding to Example 2K and 2J).

FIG. 9 shows the ratio of cryptic exon splicing to correct splicing of the STMN2 minigene in the presence of different example modified U7 snRNA constructs of the invention containing A) an antisense sequence that targets one or more exonic splice enhancers within the STMN2 cryptic exon, or B) an antisense sequence that targets the 3′-splice site of the STMN2 cryptic exon.

FIG. 10 shows the TDP-43 regulated UNC13A cryptic exon target and flanking regions thereof, annotated with splicing elements. The sequence corresponds to SEQ ID NO: 4 yo

FIG. 11 shows the TDP-43 regulated SNTM2 cryptic exon target and flanking regions thereof, annotated with splicing elements. The sequence corresponds to SEQ ID NO: 7

FIG. 12 shows the TDP-43 regulated INSR cryptic exon target and flanking regions thereof, annotated with splicing elements. The sequence corresponds to SEQ ID NO: 9

FIG. 13 shows STMN2 levels are rescued using vectorised U7 snRNPs targeting the STMN2 cryptic exon STMN2 protein levels were assessed in Doxycycline (Dox)-inducible TDP-43 SH-SY5Y cells that were either non-transduced (Control), or transduced with either, a non-targeting U7SmOPT (U7 Control), or a U7SmOPT of the invention targeting 3′ splice site (Ex. 2J) expressing lentiviral vector in the presence (TDP-43 KD+) or absence (TDP-43 KD−) of a TDP-43 knockdown. GAPDH protein levels were assessed as loading control.

FIG. 14 shows UNC13A levels are rescued using vectorised U7 snRNPs targeting the UNC13A cryptic exon in SH-SY5Y cells. UNC13A protein levels were assessed in Doxycycline (Dox)-inducible TDP-43 SH-SY5Y cells that were either non-transduced (Control), or transduced with either, a non-targeting U7SmOPT (U7 Control), or a monofunctional U7SmOPT of the invention targeting TDP-43 binding site and a 5′ splice site (Ex. 1F) expressing lentiviral vector in the presence (TDP-43 KD+) or absence (TDP-43 KD−) of a TDP-43 knockdown. GAPDH protein levels were assessed as loading control.

FIG. 15 shows RNA and protein rescue of UNC13A mis-splicing using UNC13A-targeting a U7 Construct of the invention (Ex. 1F) in Human iPSC-derived cortical neurons. Human iPSC-derived cortical neurons (i3Neurons) expressing the U7 construct were cultured. TDP-43 knockdown was achieved by treating the cells with Halo-Protac (300 nM). RNA and protein were harvested on day 11. Top) RT-PCR analysis of UNC13A splicing between exons 19 and 22 shows a rescue in splicing with the U7 construct. Bottom) Western blot analysis of UNC13A levels following treatment with the U7 construct shows a rescue of UNC13A protein.

FIG. 16: RNA and protein rescue of STMN2 mis-splicing using a STMN2-targeting U7 construct of the invention (Ex. 2J). Human iPSC-derived cortical neurons (i3Neurons) expressing the U7 constructs were cultured. TDP-43 knockdown was achieved by treating the cells with Halo-Protac (300 nM). RNA and protein were harvested on day 11. Top) Three-primer RT-PCR analysis of STMN2 splicing at between exons 1 and 2 shows a rescue in splicing with the U7 construct. Bottom) Western blot analysis of STMN2 levels following treatment with the U7 construct shows a rescue of STMN2 protein.

FIG. 17 shows the ratio of cryptic exon included to correctly spliced or total RT-qPCR levels of STMN2 (A), UNC13A (B) and INSR (C) mRNA in 293T-2×TDP-shRNA cells transfected with an STMN2 and an UNC13A minigene upon transfection with non-targeting control (Uninduced and U7 Control) or a combined vector comprising multiple constructs pMA-3x-U7SmOPT (3x-tU7SmOPT). The 3x-tU7SmOPT construct contains three U7 constructs in tandem (each comprising a different antisense sequence complementary to UNC13A, STMN2 and INSR cryptic exons) and is compared to CE/Correct ratios obtained upon transfection with a single construct targeting the same antisense sequences. Data is presented as mean ±SD relative to the ratio in non-targeting control and analyzed using ordinary one-way ANOVA with Tukey's multiple comparison test (*p<0.05, ** p<0.01, *** p<0.001, **** p<0.0001).

FIG. 18 shows the RNA rescue of UNC13A, STMN2, and INSR mis-splicing using a combined vector triple U7 construct (comprising an antisense sequence for UNC13A, for STMN2, and INSR) in SH-SY5Y neuronal cells. TDP-43 inducible shRNA knockdown SH-SY5Y cells were left untreated or treated with doxycycline 0.025 μg/mL for 5 days. The cells were then electroporated with 2 μg of U7 DNA constructs with Ingenio Electroporation Kit (Mirus) using the A-023 setting on an Amaxa II nucleofector (Lonza). The cells were then left untreated or treated with 1 μg/mL doxycycline for 5 further days before RNA extraction on day 10. RT-PCR analysis of STMN2, INSR, and UNC13A splicing shows a rescue in splicing of all three genes using the combined vector triple U7 construct. The positive control demonstrated good electroporation efficiency. PCR products were resolved on a TapeStation 4200 (Agilent).

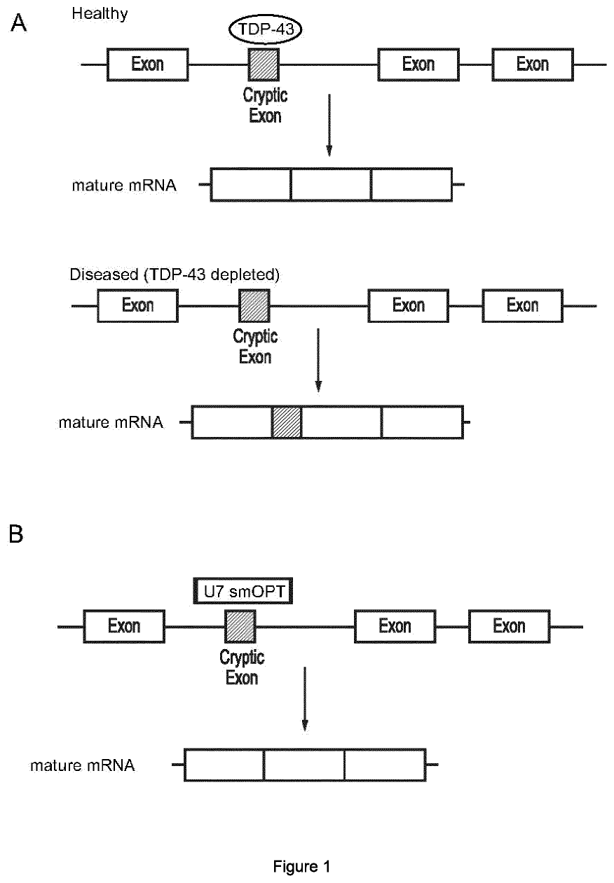

### DETAILED DESCRIPTION

The terms “treatment” and “treating” herein refer to an approach for obtaining beneficial or desired results in a subject, which includes a prophylactic benefit and a therapeutic benefit.

“Therapeutic benefit” refers to eradication, amelioration or slowing the progression of the underlying disorder being treated. Also, a therapeutic benefit is achieved with the eradication or amelioration of one or more of the physiological symptoms associated with the underlying disorder such that an improvement is observed in the subject, notwithstanding that the patient may still be afflicted with the underlying disorder.

“Prophylactic benefit” refers to delaying or eliminating the appearance of a disease or condition, delaying or eliminating the onset of symptoms of a disease or condition, slowing, halting, or reversing the progression of a disease or condition, or any combination thereof. In the context of the present invention, the prophylactic benefit or effect may involve the prevention of the condition or disease. The construct, vector, or pharmaceutical composition may be administered to a subject at risk of developing a particular disease, or to a subject reporting one or more of the physiological symptoms of a disease, even though a diagnosis of this disease may not have been made.

The term “effective amount” or “therapeutically effective amount” refers to the amount of the construct, vector, or pharmaceutical composition needed to bring about an acceptable outcome of the therapy as determined by reducing the likelihood of disease as measurable by clinical, biochemical or other indicators that are familiar to those trained in the art. The therapeutically effective amount may vary depending upon the condition, the severity of the condition, the subject, e.g., the weight and age of the subject and the mode of administration and the like, which can readily be determined by one of ordinary skill in the art.

The term “subject” refers to any suitable subject, including any animal, such as a mammal. In preferred embodiments described herein, the subject is a human.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features. The term “comprises” or “comprising” can be used interchangeably with “includes”.

“Capable of binding” as described herein refers to any nucleotide sequence that binds to the stated target region (i.e., in pre-mRNA). This can be defined as any nucleotide sequence may be substantially complementary (e.g., at least 90% complementary, or at least 95%) or 100% complementary to the target sequence and/or at least part of a splicing element which has the same number of nucleotides as the antisense sequence.

“Sequence identity” as described herein refers to the % degree of similarity between two nucleotide sequences of the same length.

“UNC13A” as defined herein is a gene that encodes for the UNC13A protein. UNC13 proteins play an important role in neurotransmitter release at synapses.

“STMN2” as defined herein is a gene that encodes for stathmin 2 protein. This protein plays a regulatory role in neuronal growth.

“INSR” as defined herein is a gene that encodes for an insulin receptor which is a member of the receptor tyrosine kinase family of proteins, where binding of insulin or other ligands to this receptor activates the insulin signalling pathway.

“ELAVL3” as defined herein refers to a gene that encodes for the neural-specific protein ELAV like RNA binding protein 3.

“CELF5” as defined herein refers to a gene that encodes for CUGBP Elav-Like Family Member 5 protein.

“TDP-43” as defined herein refers to TAR DNA Binding protein 43 (Transactive response DNA binding protein 43 kDa), which in humans is a protein encoded by the TARDBP gene. TDP-43 has been shown to bind both DNA and RNA and have multiple functions in transcriptional repression, pre-mRNA splicing and translational regulation, among other functions. Pathological TDP-43 may refer to a TDP-43 protein that is associated with a disease state. Pathological TDP-43 may be a hyper-phosphorylated, ubiquitinated or cleaved form of TDP-43, a TDP-43 form with decreased solubility, or a misfolded form of TDP-43, a mutant form of TDP-43, or a TDP-43 with altered cellular location.

A “construct” described herein has its normal meaning in the art and refers to a synthetic Nucleic acid sequence that is used to incorporate genetic material into a target cell or tissue. A construct is intended not to be a complete naturally occurring nucleic acid sequence, i.e., as found in the genome of an organism (although the construct itself may comprise component parts that are derived from naturally occurring sequences). The construct may have a maximum length, i.e., the construct may comprise less than 50,000 nucleotides, or less than 40,000 nucleotides, or less than 30,000 nucleotides, or less than 20,000 nucleotides, or in some examples, less than 10,000 nucleotides or less than 5000 nucleotides, or less than 2500 nucleotides, or less than 2000 nucleotides.

A “U7 snRNA” described herein refers to a modified variant of U7 small nuclear RNA which can form a component of the small nuclear ribonucleoprotein complex (U7 snRNP). An unmodified or wildtype U7 snRNA is any U7 snRNA that is involved in processing of replication-dependent histone pre-mRNA. A modified version of U7 snRNA refers to any U7 snRNA variant with controlled changes in the wildtype U7 snRNA such that it is not involved in the processing of replication-dependent histone-dependent pre-mRNA. This is achieved by modifying the Sm binding site of U7 snRNA (i.e., corresponding to SEQ ID NO: 353 AUUUGUCUAG in the wildtype) and modifying the sequence in the wildtype or unmodified U7 snRNA which binds to histone-downstream element within replication-dependent histone pre-mRNAs in the wildtype (SEQ ID NO: 354-AAGUGUUACAGCUCUUUUAG). The modified U7 snRNA construct may therefore not comprise a sequence identical to either SEQ ID NO: 353 or SEQ ID NO: 354. The modified U7 snRNA construct described herein instead comprises an antisense sequence that binds to a target sequence (e.g., the TDP-43 regulated cryptic exon or flanking regions thereof) in place of the histone-binding sequence in unmodified or wildtype U7 snRNA (SEQ ID NO: 354) while also comprising a modified Sm sequence. An example of a modified U7 snRNA with a modified Sm sequence is a U7 smOPT. As defined herein, “U7 smOPT” refers to a modified U7 snRNA as described above but wherein the Sm sequence has been modified to SEQ ID NO 355: AAUUUUUGGAG for the same number of nucleotides.

“Nucleotides” described herein describe the constituent parts of a nucleic acid sequence. Nucleotides comprise a nucleobase (e.g., A, G, T and C in DNA, or A, G, U and C in RNA, however other nucleobases may be used), linked to a sugar (e.g., deoxyribose in DNA, and ribose in RNA, however, other sugars may be used). In DNA and RNA, the sugars are linked by a phosphodiester backbone to form a nucleic acid sequence, however other backbones may be used.

“Complementarity” or “complementary” disclosed herein refers to Watson-Crick base pairing in nucleic acids, e.g., wherein A binds with U (or T or modified variants thereof), and wherein C binds with G (or modified variants thereof).

Reverse complement as described herein refers to the complementary strand or antisense sequence of a sequence, shown from 5′ (left) to 3′ (right).

A cell with depletion (e.g., nuclear depletion) of TDP-43 as described herein may be referred to as a “diseased cell” herein. A cell without depletion (e.g., nuclear depletion) of TDP-43 may be referred to as “healthy cell” herein.

“Splicing” as defined herein refers to the process wherein pre-mRNAs are transformed into mature mRNAs, wherein introns are removed and exons are joined together.

A “cryptic exon” as defined herein refers to a splicing variant that is incorporated into a mature mRNA, introducing frameshifts or stop codons, among other changes in the resulting mRNA. Cryptic exons are typically absent or have much reduced inclusion in the “normal” or “healthy” form of mRNA, and are usually skipped by the spliceosome, but arise in an aberrant form. A cryptic exon may otherwise be referred to as “CE”, “cryptic” “cryptic event” or “cryptic splicing event” herein or elsewhere in the art. The cryptic exon refers to the sequence which is incorrectly incorporated into mature mRNA, defined by a cryptic acceptor splice site and a cryptic donor splice site.

As defined herein, sequences comprising or defined using “T” or thymine, are intended to refer to “U” or uracil, when referring to RNA molecules and sequences defined using “U” or uracil are intended to refer to “T” when referring to DNA molecules. Sequences comprising or defined using “A”, “G”, “C”, “T” or “U” are intended to encompass modified variants of nucleotides, including nucleotides with modified nucleobases and/or modified sugars. In some embodiments, the sequences comprise only unmodified bases.

As defined herein a “splicing factor” is a protein involved in splicing, i.e., the removal of introns from mRNA so that exons are bound together.

As defined herein “a splicing repressor” is a protein involved in repressing or preventing splicing.

As defined herein, splicing elements are any part of the pre-mRNA that is involved in cryptic exon splicing. Splicing elements encompass splice sites (i.e., splice acceptor site and/or splice donor sites defining the cryptic exon), exonic sequence enhancers (ESEs) (defined below), TDP-43 binding region or TDP-43 binding motif (both defined below), or other splicing regulatory elements (i.e., site or sequences where RNA-binding proteins bind and promote splicing events).

An “exonic splice enhancer” or “ESE” defined herein may refer to an ESE that is identified by ESE finder 3.0 (http://krainer01.cshl.edu/cgi-bin/tools/ESE3/esefinder.cgi?process=home). In some embodiments, the ESE is a binding site for an SR protein, for example, a binding site or binding motif for SRSF1, SRSF2, SRSF5 or SRSF6.

A splice site, as understood in the art, is the boundary between an intron sequence and exon sequence. During splicing, the nucleotide sequence is cut at said splice sites, i.e., the nucleotide sequence is cut at the boundary between an intron sequence and exon sequence.

A splice acceptor site is a splicing site that occurs between and intron and exon, i.e., splice site immediately upstream of an exonic sequence wherein the intron is upstream of the exonic sequence. A splice acceptor site is characterised by any splice site that comprises the dinucleotide “AG” upstream of the splice site (i.e., at the end of the intron sequence which is upstream of the exon). A cryptic splice acceptor site is the splice acceptor site of the cryptic exon. Splice acceptor site and cryptic splice acceptor site may be interchangeable herein. The term splice acceptor site may be used interchangeably with the term “3-splice site” or “3-ss”

A splice donor site is a splicing site that occurs between an exon and an intron, i.e., an exonic sequence wherein the exon is upstream of the intron. A splice donor site is characterised by any splice site that comprises the dinucleotide “GU” downstream of the splice site (i.e., at the start of the intron sequence which is downstream of the exon). A cryptic splice donor site is the splice donor site of the cryptic exon. Splice donor site and cryptic splice donor site may be interchangeable herein. The term splice donor site may be used interchangeably with the term “5-splice site” or “5-ss”

“Depletion of TDP-43” or “depleted of TDP-43” as described herein, may be defined as a cell of a cell or as an average (mean) of a population of cells, with at least 20% loss of TDP-43, or at least 25% loss, or preferably at least 50% loss of TDP-43 in the cell, preferably the nucleus, as compared to a healthy cell (or as an average (mean) of a population of healthy cells) of the same type. In some examples, the term “nuclear depletion of TDP-43” can be replaced with or is interchangeable with the term “absence of binding of TDP-43 to the TDP-43 binding region”, and the term “without nuclear depletion of TDP-43” can be replaced with or is interchangeable with the term “presence of binding of TDP-43 to the TDP-43 binding region. Depletion of TDP-43 can be determined by standard methods, such as western blotting. In other embodiments, depletion may be determined by determining the presence of a STMN2 cryptic splicing event (i.e., the presence of a STMN2 cryptic exon 2a as defined herein) in a cell transcript, which may be determined by RNA-sequencing. Depletion of TDP-43 refers to depletion of “normal” or wild-type TDP-43, and may not include pathological or mutated TDP-43. Pathological TDP-43 may be a hyper-phosphorylated, ubiquitinated or cleaved form of TDP-43, a TDP-43 form with decreased solubility, or a misfolded form of TDP-43, a mutant form of TDP-43, or a TDP-43 with altered cellular location.

The term “RNA-seq” referred to herein, otherwise known as “RNA sequencing”, refers to a next-generation sequencing technology which reveals the presence and quantity of RNA in a sample which can be used to analyse the cellular transcriptome.

“Capable of modulating splicing of a TDP-43 regulated cryptic exon” as described herein refers to a construct that corrects splicing by at least partially preventing inclusion of the TDP-43 regulated cryptic exon in the mature mRNA of the cell transcript (e.g., by binding to the pre-mRNA containing the TDP-43 regulated cryptic exon).

An “anti-sense oligonucleotide” or “ASO” described herein has its normal meaning in the art and refers to an isolated (i.e., stand-alone) synthetic single stranded string of nucleic acids, typically less than 30 nucleotides in length. ASOs are used in the art as therapeutics, e.g., for targeting mRNA. They bind complementarily (‘antisense’) through Watson-Crick base pairing to a defined part of a nucleotide sequence of the pre-messenger ribonucleic acid (pre-mRNA) or mature mRNA (‘sense’) to modulate mRNA function or splicing. ASO as described herein is distinct from a modified U7 snRNA constructs described herein which instead incorporate an antisense sequence within a modified U7 snRNA construct, e.g., comprising a modified Sm sequence, more preferably a smOPT sequence.

Unless context explicitly states otherwise, it is envisaged that any embodiment described herein may be combined with any other embodiment described herein. Similarly, the features of any dependent claim (i.e., representing preferred embodiments of the present invention) may be readily combined with the features of any of the independent claims or other dependent claim or embodiments, unless context clearly dictates otherwise.

Any genomic or chromosomal position described herein refers to the position on the human genome and associated transcriptome (hg38).

When ranges are used herein, all combinations and sub-combinations of ranges and specific embodiments therein are intended to be included. The term “about” or “˜” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale.

It must be noted that as used herein and in the appended claims, the singular forms “a”, “an”, and “the” include plural referents unless the context clearly dictates otherwise.

Unless defined otherwise, all technical and scientific terms used herein have the same meanings as commonly understood by one of ordinary skill in the art to which this invention belongs. Abbreviations used herein have their conventional meaning within the chemical and biological arts, unless otherwise indicated.

### Construct

Disclosed herein is a modified U7 snRNA construct comprising at least one antisense sequence having between 16 to 30 nucleotides which are at least 90% complementary to a splicing element of a TDP-43 regulated cryptic exon sequence or flanking regions thereof and

wherein the U7 smOPT construct is capable of modulating splicing of the TDP-43 regulated cryptic exon in a cell. Flanking regions described herein may be defined as 25 nucleotides upstream and downstream of the splicing element, or in some 20 nucleotides upstream and downstream of the splicing element. In other embodiments, the splicing element or flanking regions thereof may be defined by a particular sequence for a specific TDP-43 regulated cryptic exon (e.g., SEQ ID NO: 11-40).

In some embodiments, the modified U7 snRNA construct comprises a transcription start site, e.g., in the form of an A nucleotide, at the start of the construct.

In some embodiments (and in the examples described herein), the modified U7 snRNA construct comprises the antisense sequence (i.e, which is at least 90% complementary to a TDP-43 regulated cryptic exon sequence or flanking regions thereof) downstream of the transcription start site, and preferably immediately downstream of the transcription start site.

In some embodiments, the modified U7 snRNA construct comprises a modified Sm sequence (i.e., the modified U7 snRNA is a U7 smOPT construct). Preferably, the modified Sm sequence is downstream of the antisense sequence which is at least 90% complementary to a TDP-43 regulated cryptic exon sequence or flanking regions thereof. In some embodiments, the U7 snRNA construct comprises a modified Sm sequence that has at least 80% sequence identity, (i.e., for the same number of nucleotides), to SEQ ID NO 355 AAUUUUUGGAG, or at least 85% sequence identity, or at least 90% sequence identity, or at least 100% sequence identity to SEQ ID NO 355: AAUUUUUGGAG. In preferred embodiments, the modified U7 snRNA construct is a U7 smOPT construct. The U7 smOPT construct comprises the modified Sm sequence corresponding to SEQ ID NO 355 AAUUUUUGGAG. In some embodiments, the modified U7 snRNA construct comprises a 3′ hairpin sequence downstream of the modified Sm sequence. This may be any suitable hairpin sequence. In some embodiments, the 3′ hairpin sequence has a sequence that is at least 80%, or at least 85%, or at least 90%, or at least 95%, or at least 100% identical to CAGGUUUUCUGACUUCGGUCGGAAAACCCCU (SEQ ID NO: 356). The modified U7 snRNA construct does not comprise a wildtype Sm sequence (SEQ ID NO: 353) The modified U7 snRNA construct does not comprise a binding sequence to a histone-downstream element (HDE), i.e., the modified U7 snRNA construct does not comprise the sequence corresponding to SEQ ID NO: 354. In preferred embodiments, the sequence comprising the antisense sequence which is at least 90% complementary to a TDP-43 regulated cryptic exon and flanking regions thereof is present in the modified U7 snRNA construct directly in place of the binding sequence for the histone-downstream element in wild-type U7 snRNA.

In some examples, the modified U7 snRNA construct comprises a sequence that has at least 80% sequence identity to, or at least 85%, or at least 90%, or at least 95%, or at least 100% sequence identity to any one of the example constructs described herein, i.e., corresponding to SEQ ID NO: 358, 361, 363, 365, 367, 369, 371, 373, 375, 377 (i.e., UNC13A examples) or SEQ ID NO: 379, 381, 383, 385, 387, 389, 391. 393. 395, 397, 399, 401 (i.e., STMN2 examples). Sequence identity is compared to a sequence with the same number of nucleotides.

### Antisense Sequence

The constructs described herein comprise an antisense sequence that is at least 90% complementary to a TDP-43 regulated cryptic exon or flanking regions thereof. In some embodiments, the antisense sequence is at least 91% complementary, or at least 92% complementary, or at least 93% complementary, or at least 94% complementary, or at least 95% complementary, or at least 96% complementary, or at least 97% complementary, or at least 98% complementary, or at least 99% complementary, or at least 100% complementary to a TDP-43 regulated cryptic exon or flanking regions thereof. In some embodiments, the TDP-43 regulated cryptic exon or flanking regions thereof may be defined by SEQ ID NO: 1, 2, 3, 4 (e.g., for UNC13A), SEQ ID 7 (e.g., for STMN2) or SEQ ID 9 (e.g., for INSR).

The flanking region of the TDP-43 regulated cryptic exon may be defined as the 150 nucleotides upstream and/or downstream of the cryptic exon (i.e., in intronic regions surrounding the cryptic exon sequence). In some embodiments, the flanking region may be the 100 nucleotides upstream and/or downstream of the cryptic exon, or up to 75 nucleotides upstream and/or downstream of the cryptic exon, or up to 50 nucleotides upstream and/or downstream of the cryptic exon, or up to 30 nucleotides upstream and/or downstream of the cryptic exon, or 25 nucleotides upstream and/or downstream of the cryptic exon. In some embodiments, the antisense sequence may partially overlap with the cryptic exon sequence (i.e., the antisense sequence is capable of binding to a part of the cryptic exon sequence and part of the flanking region thereof). In some embodiments, the antisense sequence is capable of binding to at least 5 nucleotides within the cryptic exon, or at least 10 nucleotides, or at least 15 nucleotides within the cryptic exon sequence. The cryptic exon sequence may be any cryptic exon sequence defined herein. In some embodiments, the antisense sequence may be capable of binding within the cryptic exon sequence. In some embodiments, the antisense sequence is at least 90%, or at least 91%, or at least 92%, or at least 93%, or at least 94%, or at least 95%, or at least 96%, or at least 97%, or at least 98%, or at least 99%, or 100% complementary to any one of SEQ ID NO: 5, 6 (short and long cryptic exon for UNC13A), SEQ ID 8 (cryptic exon of STMN2) or SEQ ID 10 (cryptic exon for INSR).

A “TDP-43 regulated cryptic exon” defined herein is a cryptic exon that is regulated by binding of TDP-43 to a TDP-43 binding region in close proximity to the cryptic exon, such that splicing of the cryptic exon is repressed. A TDP-43 regulated cryptic exon is therefore characterized as a cryptic exon that is present or increased relative to a healthy cell in the mature mRNA of a gene when there is depletion of TDP-43 in the cell and/or in the absence of TDP-43 binding, but is absent in the mature mRNA of a gene or decreased when there is no such depletion of TDP-43. A TDP-43 regulated cryptic exon is further characterized by a cryptic exon that comprises or is in close proximity to a TDP-43 binding region (defined below), wherein close proximity is defined as a region which is entirely within, partially overlaps, or is within 150 nucleotides of the cryptic exon sequence. In some embodiments, the TDP-43 binding region encompasses at least part of the cryptic exon sequence, and/or extends upstream or downstream of the cryptic exon sequence. In some embodiments, the TDP-binding region (or at least a part of the TDP-43 binding region) is within 150 nucleotides (i.e., upstream or downstream) of the cryptic exon, or within 100 nucleotides, or within 50 nucleotides, or within 25 nucleotides of the cryptic exon, or within the cryptic exon. In some embodiments, the TDP-43 binding region is upstream of the cryptic exon sequence, within the cryptic exon sequence, or downstream of the cryptic exon sequence or any combination thereof. Such cryptic exons are well-known in the art and can be readily identified in the art (e.g., by comparing the level of cryptic splicing in healthy or wildtype cells versus cells depleted by TDP-43 (i.e., in diseased cells or cells with TDP-43 knockdown or TDP-43 knockout). In some embodiments, the TDP-43 binding region comprises or is a TDP-binding motif. The TDP-43 binding motif may be as elsewhere described herein.

In some embodiments, the TDP-43 regulated cryptic exon is a cryptic exon within the following genes: AARS1, AC002310.11, AC008676.3, AC022387.2, ACTL6B, ADARB1, ADCY1, ADGRL1, AGK, AHNAK, AKT3, AL035461.3, AL360181.3, AP000662.4, ARAP3, ARHGAP22, ARHGAP23, ATAD5, ATG4B, ATP5MG, ATP8A2, ATXN1, C2orf81, CAMK2B, CAMTA1, CCDC102B, CCDC33, CDHR2, CELF5, CEP290, CEP83, CHD8, CHFR, CRLS1, CTD-2162K18.4, CYFIP2, DACH2, DACT3-AS1, DAGLA, DELE1, DGKA, DLG5, DLGAP1, DNAJC12, DNMT3A, DOCK1, DPF1, DUXAP9, EIF2A, ELAVL3, EP400, EPB41L4A, EPS8L2, FADS2, FAM114A2, FAM156A, FIRRE, FKBP14-AS1, FRYL, G3BP1, GALNT12, GATA2, GPSM2, GREB1, GRIN2D, GSTCD, HAUS2, HDGFL2, ICA1, IGSF21, IK, IL15, INSR, INTS11, IQCE, IQCK, ISYNA1, ITGA7, ITPR3, KALRN, KCNQ2, KCNT1, KIAA1211, KIAA1217, KIF21A, KLC1, KNDC1, L3MBTL1, LINC01322, LINC01503, LINGO1, LRP1B, LRP8, LTBP2, MACROD1, MADD, MANBAL, MAP2K6, MBP, MC1R, MCM9, MED13L, MEIS2, MGAT5B, MIER3, MMAA, MRPL34, NBPF9, NIPSNAP3B, NTRK2, NUP188, PAOX, PATJ, PCDH11X, PDCD6, PDE2A, PHF2, PLEKHG2, PLEKHM2, PRUNE2, PTPN13, PTPN21, PUDP, PUS7L, RBMXL1, RFLNA, RHOQ, RP1-138B7.8, RP11-108K14.8, RP11-411B6.6, RP11-47909.4, RP11-505D17.1, RP11-61L23.2, RP11-73M18.2, RP5-967N21.13, RSF1, SEC31B, SEPT7P2, SEPTIN6, SEPTIN7P2, SERGEF, SETD5, SGMS1, SIPA1L3, SLC24A3, SLC25A14, SLC2A11, SLC35G1, SLC41A2, SPATS2, SPIN1, STMN2, STRA6, STXBP5L, SYNE1, SYNJ2, SYT7, TAF6, TAFA2, TEX9, TGFB3, THUMPD3-AS1, TMEM175, TMEM189, TPRA1, TRAPPC12, TRIO, TRRAP, TTC39C-AS1, TTTY14, TXLNGY, UNC13A, USP10, WARS2, WASL, WDR19, WWOX, ZBTB18, ZCCHC4, ZFAT, ZNF202, ZNF236, ZNF382, ZNF420, ZNF423, ZNF429, ZNF527, ZNF571-AS1, ZNF583, ZNF598, ZNF81, ZNF814, ZNF826P, ZRANB3.

In some embodiments, the TDP-43 regulated cryptic exon is a cryptic exon within UNC13A, STMN2, INSR, ELALV3, G3BP1, AARS1, CELF5, CAMK2B or UNC13B, preferably wherein the antisense sequence is at least 90% complementary (or at least 95% complementary, or at least 100% complementary) to one of SEQ ID NO: 1-40 or SEQ ID 431-454, or SEQ ID NO: 11-40 or SEQ ID NO: 437-454.

In some embodiments, the TDP-43 regulated cryptic exon is a cryptic exon within UNC13A, STMN2 or INSR, preferably wherein the antisense sequence is at least 90% complementary (or at least 95% complementary, or at least 100% complementary) to one of SEQ ID NO: 1-40, or SEQ ID NO: 11-40.

In some embodiments, the TDP-43 regulated cryptic exon is selected from a UNC13A cryptic exon, a TDP-43 regulated STMN2 cryptic exon, a TDP-43 regulated INSR cryptic exon, a TDP-43 regulated ELAVL3 cryptic exon, a TDP-43 regulated G3BP1 cryptic exon, an AARS1 TDP-43 regulated cryptic exon, a TDP-43 regulated CELF5 cryptic exon, a TDP-43 regulated CAMK2B cryptic exon, or a TDP-43 UNC13B cryptic exon, preferably wherein the antisense sequence comprises a sequence that is at least 90%, or at least 95%, or at least 100% complementary to any one of SEQ ID NO: 1, 2, 3, 4, 7, 9, 431, 432, 433, 434, 435, 436. In some embodiments, the TDP-43 regulated cryptic exon is selected from a UNC13A cryptic exon, a TDP-43 regulated STMN2 cryptic exon, a TDP-43 regulated INSR cryptic exon, a TDP-43 regulated ELAVL3 cryptic exon, or a TDP-43 regulated CELF5 cryptic exon, preferably wherein the antisense sequence comprises a sequence that is at least 90%, or at least 95%, or at least 100% complementary to any one of SEQ ID NO: 1, 2, 3, 4, 7, 9, 431 or 434. In some examples described herein, the TDP-43 regulated cryptic exon is a TDP-43 regulated UNC13A cryptic exon, a TDP-43 regulated STMN2 cryptic exon or a TDP-43 regulated INSR cryptic exon. In some examples, the antisense sequence comprises a sequence that is at least 90%, or at least 95%, or at least 100% complementary to any one of SEQ ID NO: 1, 2, 3, 4, 7, 9.

As defined herein, the TDP-43 binding region is defined as a sequence that is capable of binding to TDP-43. This term may be used interchangeably with the term “TDP-43 binding domain” or “TDP-43 binding site” and may encompass a sequence with a “TDP-43 binding motif”. The TDP-43 binding region is typically characterised or encompasses a “UG rich” sequence or region. In some embodiments, the “UG” rich region may be defined, and the TDP-43 binding region may comprise a region of at least 6 nucleotides, or preferably at least 10 nucleotides, or at least 20 nucleotides, with a statistically significant enrichment of UG dinucleotides and/or UGNNUG hexanucleotides, wherein N is A, U, C or G. In some embodiments, the TDP-43 binding region comprises a region of at least 6 nucleotides (e.g., 6 to 1000 nucleotides, or 6 to 150 nucleotides), with a statistically significant enrichment of UG dinucleotides and/or UGNNUG hexanucleotides, wherein N is A, U, C or G, wherein statistically significant enrichment is defined as a probability of less than 0.2% that a random sequence of nucleotides of equal length would feature an equal number of UG dinucleotides and/or UGNNUG hexanucleotides. In some embodiments, the statistically significant enrichment is defined as a probability of less than or equal to 0.15% that a random sequence of nucleotides of equal length would feature an equal number of UG dinucleotides and/or UGNNUG hexanucleotides, or less than or equal to 0.1%, or less than or equal to 0.05%, or less than or equal to 0.01%, or less than or equal to 0.003%, or equal or less than 0.001%, or equal or less than 0.0003%, or equal or less than 0.0001%, or equal or less than 1×10⁻⁵, or of less than or equal to 1×10⁻⁶, or of less than or equal to 1×10⁻⁷, or of less than or equal to 1×10⁻⁸, or of less than or equal to 1×10⁻⁹, or less than or equal to 1×10⁻¹⁰. These definitions cover both short sequences or regions which are highly enriched for UG, and longer sequences which are broadly enriched for UG, both of which are shown to be preferentially bound by TDP-43. In some embodiments, the TDP-43 binding region comprises a sequence that is enriched with UG dinucleotides. In some embodiments, an enrichment of UG dinucleotides may be described as a TDP-binding motif and is defined as a sequence comprising at least 6 nucleotides with 100% UG dinucleotides (i.e., UGUGUG), or one or more region with at least 6 nucleotides with 100% UG dinucleotides. In some embodiments, an enrichment of UG dinucleotides is defined as a sequence comprising at least 8 nucleotides (or one or more region with at least 8 nucleotides) with at least 80% UG dinucleotides, or at least 85%, or at least 90%, or at least 95%, or 100% UG dinucleotides. In some embodiments, an enrichment of UG dinucleotides is defined as a sequence which comprises at least 10 nucleotides (or one or more region with at least 10 nucleotides) with at least 60% UG dinucleotides, or at least 65%, or at least 70%, or at least 75%, or at least 80%, or at least 85%, or at least 90%, or at least 95%, or at least 100% UG dinucleotides. In some embodiments, an enrichment of UG dinucleotides is defined as a sequence that comprises at least 15 nucleotides (or one or more region with at least 15 nucleotides) with at least 53% UG dinucleotides, or at least 60%, or at least 65%, or at least 70%, or at least 75%, or at least 80%, or at least 85%, or at least 90%, or at least 95%, or 100% UG dinucleotides). In some embodiments, the TDP-43 binding region comprises a sequence that comprises at least one UGUGUG motif, or at least one UGUGUGUG motif. However, while TDP-43 is capable of binding a large variety of different sequences that are UG-rich, the TDP-43 binding region does not have to bind a pure UG-repeat. This is in part due to the protein's lack of contact with some RNA residues within its binding footprint, and in part due to multivalent protein-protein interactions which enhance binding to large regions of UG-rich RNA. This means that in some embodiments, the TDP-43 binding region may not require any “pure” UG-repeats or motifs, such as the TDP-43 binding region in UNC13A. In some embodiments, the TDP-43 binding region may be a well-known, described annotated binding region. For example, the sequence characteristics which promote binding of TDP-43 are described in Lukavsky et al., 2013 (NSMB, 20, pages 1443-1449) which is incorporated herein by reference. The TDP-43 binding region may be or may have been previously identified by transcriptome mapping of TDP-43 on the human genome, for example, as determined by immunoprecipitation, for example, iCLIP (individual-nucleotide resolution UV Cross-Linking and Immunoprecipitation).

The antisense sequence described herein may comprise or consist of from 16 to 30 nucleotides. In some embodiments, the antisense sequence is between 16 and 26 nucleotides, or between 17 and 23 nucleotides, or between 18 and 22 nucleotides. In some embodiments, the antisense sequence comprises or consists of 16 nucleotides, or 17 nucleotides, or 18 nucleotides, or 19 nucleotides, or 20 nucleotides, or 21 nucleotides, or 22 nucleotides, or 23 nucleotides, or 24 nucleotides, or 25 nucleotides, or 26 nucleotides, or 27 nucleotides, or 28 nucleotides, or 29 nucleotides or 30 nucleotides. In some embodiments, the antisense sequence comprises at least 16 nucleotides, or at least 17 nucleotides, or at least 18 nucleotides, or at least 19 nucleotides, or at least 20 nucleotides, or at least 21 nucleotides, or at least 22 nucleotides, or at least 23 nucleotides, or at least 24 nucleotides, or at least 25 nucleotides, or at least 26 nucleotides, or at least 27 nucleotides, or at least 28 nucleotides, or at least 29 nucleotides. In some embodiments, the antisense sequence comprises less than 30 nucleotides, or less than 29 nucleotides, or less than 28 nucleotides, or less than 27 nucleotides, or less than 26 nucleotides, or less than 25 nucleotides, or less than 24 nucleotides, or less than 23 nucleotides, or less than 22 nucleotides, or less than 21 nucleotides, or less than 20 nucleotides, or less than 19 nucleotides, or less than 18 nucleotides, or less than 17 nucleotides. The longer the antisense sequence, the more efficiently the modified U7 snRNA construct is found to bind and the more effective the construct is as a steric block, however, this comes with a trade-off of an increased tendency for off-target binding.

In some embodiments, the construct may comprise more than one antisense sequence, for example, two or more antisense sequences, that are at least 90% complementary to a TDP-43 regulated cryptic exon or flanking regions thereof. These antisense sequences may be capable of binding to different splicing elements.

In some embodiments, the antisense sequence is capable of binding (i.e., at least partially) to a splicing element of the cryptic exon sequence, or two or more splicing elements of the cryptic exon sequence. In some embodiments, the splicing element is selected from a splice site, a TDP-43 binding region (e.g., a TDP-43 binding motif), or an exonic splice enhancer. In some embodiments, the antisense sequence is capable of binding, at least partially, to a splicing element of the cryptic exon sequence, but may also bind to a flanking region upstream or downstream of the splicing element. The flanking regions may include the 20 nucleotides upstream of downstream of the splicing element, optionally the 15 nucleotides upstream or downstream of the splicing element, or the 10 nucleotides upstream or downstream of the splicing element, or the 5 nucleotides upstream or downstream of the splicing element. In some embodiments, (e.g., for some embodiments where the antisense sequence is capable of binding to a TDP-43 binding region), the antisense sequence is capable of binding completely to the splicing element (i.e., within the TDP-43 binding region, or completely overlapping with the ESE).

In some embodiments, the portion of the antisense sequence that is capable of binding to the splicing element is closer to the 3′-end of the antisense sequence. In some embodiments, the portion of the antisense sequence that is capable of binding to the splicing element is within 7 nucleotides, or 6 nucleotides, or 5 nucleotides, or 4 nucleotides, or 3 nucleotides, or 2 nucleotides from the 3′ end of the antisense sequence. In some embodiments, the portion of the antisense sequence that is capable of binding to the splicing element is closer to the 5′-end of the antisense sequence. In some embodiments, the portion of the antisense sequence that is capable of binding to the splicing element is within 7 nucleotides, or 6 nucleotides, or 5 nucleotides, or 4 nucleotides, or 3 nucleotides, or 2 nucleotides from the 5′ end of the antisense sequence.

In some embodiments, the splicing element is a splice site, (i.e., the antisense sequence is capable of binding (in other words, overlaps with) a splice site of the cryptic exon, more particularly wherein the antisense sequence overlaps with at least one nucleotide upstream or downstream of the splice site). In some embodiments, the antisense sequence is capable of binding to at least 2 nucleotides, or at least 3 nucleotides, or at least 4 nucleotides, or at least 5 nucleotides, or at least 6 nucleotides, or at least 7 nucleotides, or at least 8 nucleotides upstream and/or downstream of the splice site.

In some embodiments, the antisense sequence is capable of binding to a splice site (i.e., and flanking regions thereof), preferably wherein the antisense sequence is at least 90% complementary, or at least 95% complementary, or at least 100% complementary to any one of SEQ ID NO 11, 19, 20, 21, 22, 31, 437, 441, 443, 446, 450 or 452.

In some embodiments, the splicing element may be a 3′-splice site (i.e., a splice acceptor site). In some embodiments, the antisense sequence is capable of binding (in other words, overlaps with) to the “ag” dinucleotide upstream of the splice acceptor site.

In some embodiments, the splicing element may be a 5′ splice site (i.e., a splice donor site). In some embodiments, the antisense sequence is capable of binding (in other words, overlaps with) the “gu” dinucleotide downstream of the splice donor site.

In some embodiments, the splicing element is a TDP-43 binding region, and the antisense sequence is capable of binding to at least a portion of the TDP-43 binding region. In some embodiments, the antisense sequence may bind to at least a portion of the TDP-43 binding region and a flanking region thereof (i.e., as defined as 20 nucleotides upstream or downstream of the TDP-43 binding region, optionally the 15 nucleotides upstream or downstream of the TDP-43 binding region, or the 10 nucleotides upstream or downstream of the TDP-43 binding region, or the 5 nucleotides upstream or downstream of the TDP-43 binding region). In some embodiments, the antisense sequence binds to at least 5 nucleotides, or least 7 nucleotides, or at least 10 nucleotides, or at least 15 nucleotides of the TDP-43 binding region, or completely overlaps with (i.e., is contained within) the TDP-43 binding region. In some embodiments, the TDP-43 binding region comprises a sequence of at least 6 nucleotides, or preferably at least 10 nucleotides, with a statistically significant enrichment of UG dinucleotides and/or UGNNUG hexanucleotides, wherein N is A, U, C or G, wherein statistically significant enrichment is defined as a probability of less than 0.2% that a random sequence of nucleotides of equal length would feature an equal number of UG dinucleotides and/or UGNNUG hexanucleotides, or preferably wherein statistically significant enrichment is defined as a probability of less than 0.05% that a random sequence of nucleotides of equal length would feature an equal number of UG dinucleotides and/or UGNNUG hexanucleotides. In other embodiments, the TDP-43 binding region may be as according to any other definition as described herein. In some embodiments, the TDP-43 binding region may comprise or is a TDP-43 binding motif as described herein. In some embodiments, the TDP-43 binding region may comprise or is a TDP-43 binding motif as described herein. In some embodiments, the TDP-43 binding region or TDP-43 binding region and flanking region thereof is defined by SEQ ID NO: 12, 13, 23, 23, 24, 25, 26, 32, 33, 438, 439, 440, 442, 444, 445, 447, 448, 449, 451, 453 or 454.

In some embodiments, the antisense sequence is capable of binding to one or more exonic splice enhancers (ESE) (i.e., and flanking regions thereof) as defined by ESE finder 3.0, e.g., using the SR Protein matrix library. In some embodiments, the following thresholds are used when using ESE finder 3.0, when selecting the SR Protein matrix library, SRSF1-1.956, SRSF2-2.383, SRSF5 2.67 and SRSF6-2.676. The ESEs defined by ESE finder 3.0 may be as described in the reference “An increased specificity score matrix for the prediction of SF2/ASF-specific exonic splicing enhancers. Hum. Mol. Genet. 15 (16): 2490-2508, which is incorporated herein by reference. The ESE may be an SR protein binding site, i.e., selected from SRSF1, SRSF2, SRSF5 or SRSF6.

In some embodiments, the ESE may be a SRSF1 binding site. In some embodiments, the SRSF1 binding site may comprise a motif selected from CACACGA, CACACGU, CACACGG, CAGACGA, CAGACGU, CAGACGG, CACAGGA, CACAGGU, CACAGGG, CAGAGGA, CAGAGGU, CAGAGGG, CGCACGA, CGCACGU, CGCACGG, CGGACGA, CGGACGU, CGGACGG, CGCAGGA, CGCAGGU, CGCAGGG, CGGAGGA, CGGAGGU, CGGAGGG, CUCACGA, CUCACGU, CUCACGG, CUGACGA, CUGACGU, CUGACGG, CUCAGGA, CUCAGGU, CUCAGGG, CUGAGGA, CUGAGGU, CUGAGGG, CACCCGA, CACCCGU, CACCCGG, CAGCCGA, CAGCCGU, CAGCCGG, CACCGGA, CACCGGU, CACCGGG, CAGCGGA, CAGCGGU, CAGCGGG, CGCCCGA, CGCCCGU, CGCCCGG, CGGCCGA, CGGCCGU, CGGCCGG, CGCCGGA, CGCCGGU, CGCCGGG, CGGCGGA, CGGCGGU, CGGCGGG, CUCCCGA, CUCCCGU, CUCCCGG, CUGCCGA, CUGCCGU, CUGCCGG, CUCCGGA, CUCCGGU, CUCCGGG, CUGCGGA, CUGCGGU, or CUGCGGG.

In some embodiments, the ESE may be a SRSF2 binding site. In some embodiments, the SRSF2 binding site may comprise a motif selected from GGWWNCWG, GAWWNCWG, GGWWNGWG, GAWWNGWG where N is A, U, C or G and Wis U or A, or wherein the SRSF2 binding site is GGCCNCUG, GACCNCUG, GGUCNCUG, GAUCNCUG, GGCUNCUG, GACUNCUG, GGUUNCUG, GAUUNCUG, GGCCNCUA, GACCNCUA, GGUCNCUA, GAUCNCUA, GGCUNCUA, GACUNCUA, GGUUNCUA, GAUUNCUA, GGCCNCCG, GACCNCCG, GGUCNCCG, GAUCNCCG, GGCUNCCG, GACUNCCG, GGUUNCCG, GAUUNCCG, GGCCNCCA, GACCNCCA, GGUCNCCA, GAUCNCCA, GGCUNCCA, GACUNCCA, GGUUNCCA, GAUUNCCA.

In some embodiments, the ESE may be a SRSF5 binding site. In some embodiments, the SRSF5 binding site may comprise a motif selected from UCWCWGG, CCWCWGG, UCWCWCG, CCWCWCG, UCWCWGC, CCWCWGC, UCWCWCC, CCWCWCC, UCWCWAG, CCWCWAG, UCWCWAG, CCWCWAG, UCWCWAC, CCWCWAC, UCWCWAC, CCWCWAC, where W is A or U.

In some embodiments, the ESE may be a SRSF6 binding site. In some embodiments, the SRSF6 binding site may comprise a motif selected from UGCGUC, CGCGUC, UACGUC, CACGUC, UGCAUC, CGCAUC, UACAUC, CACAUC, UGCGGC, CGCGGC, UACGGC, CACGGC, UGCAGC, CGCAGC, UACAGC, CACAGC, UGCGUA, CGCGUA, UACGUA, CACGUA, UGCAUA, CGCAUA, UACAUA, CACAUA, UGCGGA, CGCGGA, UACGGA, CACGGA, UGCAGA, CGCAGA, UACAGA, or CACAGA.

In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-352. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-352. In some embodiments, the nucleotide sequence may consist of 17, 18, 19, 20, 21, 22, 23, 24, 25, 26, 27 28, 29 or 30 nucleotides.

In some embodiments, the antisense sequence comprises a sequence that is at least 80% identical, or at least 85% identical, or at least 90% identical, or at least 95% identical or at least 100% identical to any one of SEQ ID NO 359, 362, 364, 366, 368, 370, 372, 374. 376, 378, 380, 382, 384, 386, 388, 390, 392, 394, 396, 398, 400 or 402, for a sequence with the same number of nucleotides. In some embodiments, the antisense sequence comprises sequence of at least 16 nucleotides (or 16 nucleotides) that is at least 80% identical, or at least 85% identical, or at least 90% identical, or at least 95% identical or at least 100% identical to at least a portion of any one of SEQ ID NO 359, 362, 364, 366, 368, 370, 372, 374. 376, 378, 380, 382, 384, 386, 388, 390, 392, 394, 396, 398, 400 or 402, i.e., for the same number of nucleotides. In some embodiments, the antisense sequence comprises sequence of 17 nucleotides that is at least 80% identical, or at least 85% identical, or at least 90% identical, or at least 95% identical or at least 100% identical to a 17 nucleotide sequence of any one of SEQ ID NO 359, 362, 364, 366, 368, 370, 372, 374. 376, 378, 380, 382, 384, 386, 388, 390, 392, 394, 396, 398, 400 or 402. In some embodiments, the antisense sequence comprises sequence of 18 nucleotides that is at least 80% identical, or at least 85% identical, or at least 90% identical, or at least 95% identical or at least 100% identical to an 18 nucleotide sequence of any one of SEQ ID NO 359, 362, 364, 366, 368, 370, 372, 374. 376, 378, 380, 382, 384, 386, 388, 390, 392, 394, 396, 398, 400 or 402. In some embodiments, the antisense sequence comprises sequence of 19 nucleotides that is at least 80% identical, or at least 85% identical, or at least 90% identical, or at least 95% identical or at least 100% identical to a 19 nucleotide sequence of any one of SEQ ID NO 359, 362, 364, 366, 368, 370, 372, 374. 376, 378, 380, 382, 384, 386, 388, 390, 392, 394, 396, 398, 400 or 402. In some embodiments, the antisense sequence comprises sequence of 20 nucleotides that is at least 80% identical, or at least 85% identical, or at least 90% identical, or at least 95% identical or at least 100% identical to a 20 nucleotide sequence of any one of SEQ ID NO 359, 362, 364, 366, 368, 370, 372, 374. 376, 378, 380, 382, 384, 386, 388, 390, 392, 394, 396, 398, 400 or 402. In some embodiments, the antisense sequence comprises sequence of 21 nucleotides that is at least 80% identical, or at least 85% identical, or at least 90% identical, or at least 95% identical or at least 100% identical to a 21 nucleotide sequence of any one of SEQ ID NO 359, 362, 364, 366, 368, 370, 372, 374. 376, 378, 380, 382, 384, 386, 388, 390, 392, 394, 396, 398, 400 or 402. In some embodiments, the antisense sequence comprises sequence of 22 nucleotides that is at least 80% identical, or at least 85% identical, or at least 90% identical, or at least 95% identical or at least 100% identical to a 22 nucleotide sequence of any one of SEQ ID NO: 359, 362, 364, 366, 368, 370, 372, 374. 376, 378, 380, 382, 384, 386, 388, 390, 392, 394, 396, 398, 400 or 402.

### UNC13A

In some embodiments, the TDP-43 regulated cryptic exon is an UNC13A cryptic exon. The TDP-43 regulated UNC13A cryptic exon is the cryptic exon between exons 20 and 21 in the human UNC13A gene. In some embodiments, the antisense sequence is at least 90% complementary (or at least 95%, or at least 100% complementary) to SEQ ID NO: 1 or SEQ ID NO: 2. In some embodiments, the antisense sequence is at least 90% complementary (or at least 95%, or at least 100% complementary) with SEQ ID NO: 3 or 4. In some embodiments, the antisense sequence is at least 90% complementary (or at least 95%, or at least 100% complementary) to SEQ ID NO: 5. In some embodiments, the antisense sequence is at least 90% complementary (or at least 95%, or at least 100% complementary) to SEQ ID NO: 6.

In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-260. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-260. In some embodiments, the nucleotide sequence may consist of 17, 18, 19, 20, 21, 22, 23, 24, 25, 26, 27 28, 29 or 30 nucleotides.

In some embodiments, the antisense sequence is capable of binding to a UNC13A splice site (i.e., and flanking regions thereof), preferably wherein the antisense sequence is at least 90% complementary, or at least 95% complementary, or at least 100% complementary to any one of SEQ ID NO 19, 20, 21 or 22. In some embodiments, the antisense sequence is a 16 nucleotide sequence which is at least 90% complementary, or at least 95% complementary, or at least 100% complementary to any one of SEQ ID NO 19-22, or a 17 nucleotide sequence which is at least 90% complementary, or at least 95% complementary, or at least 100% complementary to any one of SEQ ID NO 19-22, a 18 nucleotide sequence which is at least 90% complementary, or at least 95% complementary, or at least 100% complementary to any one of SEQ ID NO 19-22, a 19 nucleotide sequence which is at least 90% complementary, or at least 95% complementary, or at least 100% complementary to any one of SEQ ID NO 19-22, or a 20 nucleotide sequence which is at least 90% complementary, or at least 95% complementary, or at least 100% complementary to any one of SEQ ID NO 19-22, a 21 nucleotide sequence which is at least 90% complementary, or at least 95% complementary, or at least 100% complementary to any one of SEQ ID NO 19-22, or a 22 nucleotide sequence which is at least 90% complementary, or at least 95% complementary, or at least 100% complementary to any one of SEQ ID NO 19-22. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-152. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-152. In some embodiments, the nucleotide sequence may consist of 17, 18, 19, 20, 21, 22, 23, 24, 25, 26, 27 28, 29 or 30 nucleotides.

In some embodiments, the antisense sequence is capable of binding to a UNC13A splice donor site (i.e., a 5′ splice site), i.e., and flanking regions thereof, preferably wherein the antisense sequence is at least 90% complementary, or at least 90% complementary, or at least 100% complementary to SEQ ID NO: 21-22. In some embodiments, the antisense sequence is capable of binding to one of more of the motifs GAUGG/G, AUGG/GU, UGG/GUG, GG/GUGA, G/GUGAG of the UNC13A cryptic exon and flanking regions, wherein/represents the cryptic exon/intron boundary of the UNC13A cryptic exon. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 136-152. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 136-152. In some embodiments, the nucleotide sequence may consist of 17, 18, 19, 20, 21, 22, 23, 24, 25, 26, 27 28, 29 or 30 nucleotides. In some embodiments, the antisense sequence is capable of binding to a UNC13A splice acceptor site (i.e., a 3′-splice site), i.e., and flanking regions thereof, preferably wherein the antisense sequence is at least 95% complementary, or at least 90% complementary, or at least 100% complementary to one of SEQ ID NO: 19 or SEQ ID NO: 20. In some embodiments, the antisense sequence is capable of binding to one of more of the motifs UCCAG/C, CCAG/CC, CAG/CCC, AG/CCCU or G/CCCUA the UNC13A cryptic exon and flanking regions, wherein/represents the intron/cryptic exon boundary of the UNC13A cryptic exon. In some embodiments, the antisense sequence is capable of binding to one of more of the motifs UCCAG/C, CCAG/CU, CAG/CUG, AG/CUGC, G/CUGCC in the UNC13A cryptic exon and flanking regions, wherein/represents the intron/cryptic exon boundary of the UNC13A cryptic exon. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-135. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-135. In some embodiments, the nucleotide sequence may consist of 17, 18, 19, 20, 21, 22, 23, 24, 25, 26, 27 28, 29 or 30 nucleotides.

In some embodiments, the antisense sequence is capable of at least partially binding to the TDP-43 binding region of the UNC13A cryptic exon, i.e., and/or flanking regions thereof, preferably wherein the antisense sequence is at least 90% complementary, or at least 95% complementary, or at least 100% complementary to any one of SEQ ID NO 23, 24, 25 or 26. In some embodiments, the antisense sequence is a 16 nucleotide sequence which is at least 90% complementary, or at least 95% complementary, or at least 100% complementary to SEQ ID NO 23, 24, 25 or 26, or a 17 nucleotide sequence which is at least 90% complementary, or at least 95% complementary, or at least 100% complementary to any one of SEQ ID NO 23, 24, 25 or 26, a 18 nucleotide sequence which is at least 90% complementary, or at least 95% complementary, or at least 100% complementary to any one of SEQ ID NO 23, 24, 25 or 26, a 19 nucleotide sequence which is at least 90% complementary, or at least 95% complementary, or at least 100% complementary to any one of SEQ ID NO 23, 24, 25 or 26, or a 20 nucleotide sequence which is at least 90% complementary, or at least 95% complementary, or at least 100% complementary to any one of SEQ ID NO 23, 24, 25 or 26, a 21 nucleotide sequence which is at least 90% complementary, or at least 95% complementary, or at least 100% complementary to any one of SEQ ID NO 23, 24, 25 or 26, or a 22 nucleotide sequence which is at least 90% complementary, or at least 95% complementary, or at least 100% complementary to any one of SEQ ID NO 23, 24, 25 or 26. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 153-260. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 153-260. In some embodiments, the nucleotide sequence may consist of 17, 18, 19, 20, 21, 22, 23, 24, 25, 26, 27 28, 29 or 30 nucleotides.

In some embodiments, the antisense sequence is capable of at least partially binding to an exonic sequence enhancer in the UNC13A cryptic exon, defined by ESE finder 3.0 using the SR Protein matrix library, preferably wherein the antisense sequence is at least 90% complementary, or at least 95% complementary, or at least 100% complementary to any one of SEQ ID NO: 27, 28, 29 or 30. In some embodiments, the antisense sequence is a 16 nucleotide sequence which is at least 90% complementary, or at least 95% complementary, or at least 100% complementary to any one of SEQ ID NO 27, 28, 29 or 30, or a 17 nucleotide sequence which is at least 90% complementary, or at least 95% complementary, or at least 100% complementary to any one of SEQ ID NO 27, 28, 29 or 30, a 18 nucleotide sequence which is at least 90% complementary, or at least 95% complementary, or at least 100% complementary to any one of SEQ ID NO 27, 28, 29 or 30, a 19 nucleotide sequence which is at least 90% complementary, or at least 95% complementary, or at least 100% complementary to any one of SEQ ID NO 27, 28, 29 or 30, or a 20 nucleotide sequence which is at least 90% complementary, or at least 95% complementary, or at least 100% complementary to any one of SEQ ID NO 27, 28, 29 or 30, a 21 nucleotide sequence which is at least 90% complementary, or at least 95% complementary, or at least 100% complementary to any one of SEQ ID NO 27, 28, 29 or 30, or a 22 nucleotide sequence which is at least 90% complementary, or at least 95% complementary, or at least 100% complementary to any one of SEQ ID NO 27, 28, 29 or 30.

In some embodiments, the exonic sequence enhancer is an SRSF1 binding site and the antisense sequence is capable of binding (i.e., complementary to or overlapping with) the motif CUCAGGA within the UNC13A cryptic exon sequence, preferably wherein the antisense sequence is at least 90% complementary, or at least 95% complementary, or at least 100% complementary to SEQ ID NO: 27. In some embodiments, the exonic sequence enhancer is an SRSF2 binding site and the antisense sequence is capable of binding (i.e., i.e., complementary to or overlapping with) the motif GUUUCCUG within the UNC13A cryptic exon sequence, preferably wherein the antisense sequence is at least 90% complementary, or at least 95% complementary, or at least 100% complementary to SEQ ID NO: 28. In some embodiments, the exonic sequence enhancer is an SRSF5 binding site and the antisense sequence is capable of binding (i.e., complementary to or overlapping with) the motif ACUCAGG within the UNC13A cryptic exon sequence, preferably wherein the antisense sequence is at least 90% complementary, or at least 95% complementary, or at least 100% complementary to SEQ ID NO: 28. In some embodiments, the exonic sequence enhancer is an SRSF6 binding site and the antisense sequence is capable of binding (i.e., complementary to) the motif UGUGUC within the UNC13A cryptic exon sequence, preferably wherein the antisense sequence is at least 90% complementary, or at least 95% complementary, or at least 100% complementary to SEQ ID NO: 29. In some embodiments, the antisense sequence is capable of binding (i.e., complementary to or overlapping with) both the SRSF5 binding site (ACUCAGG), and the SRSF1 binding site (CUCAGGA) in the UNC13A cryptic exon, preferably wherein the antisense sequence is at least 90% complementary, or at least 95% complementary, or at least 100% complementary to SEQ ID NO: 28.

In some embodiments, the antisense sequence comprises a sequence that is at least 80% identical, or at least 85% identical, or at least 90% identical, or at least 95% identical or at least 100% identical to any one of SEQ ID NO 359, 362, 364, 366, 368, 370, 372, 374, 376 or 378, for a sequence with the same number of nucleotides.

### STMN2

In some embodiments, the TDP-43 regulated cryptic exon is a STMN2 cryptic exon. The TDP-43 regulated STMN2 cryptic exon corresponds to exon 2a in the human STMN2 gene. In some embodiments, the antisense sequence is at least 90% complementary (or at least 95%, or at least 100% complementary) to SEQ ID NO: 7. In some embodiments, the TDP-43 regulated cryptic exon is a STMN2 cryptic exon. The TDP-43 regulated STMN2 cryptic exon corresponds to exon 2a in the human STMN2 gene. In some embodiments, the antisense sequence is at least 90% complementary (or at least 95%, or at least 100% complementary) to SEQ ID NO: 8.

In some embodiments, the antisense sequence is capable of binding to the STMN2 3′ splice site (i.e., splice acceptor site), i.e., and flanking regions thereof, preferably wherein the antisense sequence is at least 90% complementary, or at least 90% complementary, or at least 100% complementary to SEQ ID NO 11. In some embodiments, the antisense sequence is a 16 nucleotide sequence which is at least 90% complementary, or at least 95% complementary, or at least 100% complementary to of SEQ ID NO 11, or a 17 nucleotide sequence which is at least 90% complementary, or at least 95% complementary, or at least 100% complementary to SEQ ID NO 11, a 18 nucleotide sequence which is at least 90% complementary, or at least 95% complementary, or at least 100% complementary to SEQ ID NO 11, a 19 nucleotide sequence which is at least 90% complementary, or at least 95% complementary, or at least 100% complementary to any one of SEQ ID NO 11, or a 20 nucleotide sequence which is at least 90% complementary, or at least 95% complementary, or at least 100% complementary to SEQ ID NO 11, a 21 nucleotide sequence which is at least 90% complementary, or at least 95% complementary, or at least 100% complementary to SEQ ID NO 11, or a 22 nucleotide sequence which is at least 90% complementary, or at least 95% complementary, or at least 100% complementary to SEQ ID NO 11. In some embodiments, the antisense sequence is capable of binding to one of more of the motifs UGCAG/G, GCAG/GA, CAG/GAC, AG/GACU or G/GACUC in the STMN2 cryptic exon and flanking regions, wherein/represents the intron/cryptic exon boundary of the STMN2 cryptic exon. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-59. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-59. In some embodiments, the nucleotide sequence may consist of 17, 18, 19, 20, 21, 22, 23, 24, 25, 26, 27 28, 29 or 30 nucleotides.

In some embodiments, the antisense sequence is capable of at least partially binding to the TDP-43 binding region, or TDP-43 binding motif, of the STMN2 cryptic exon, i.e., and flanking regions thereof, preferably wherein the antisense sequence is at least 90% complementary, or at least 95% complementary, or at least 100% complementary to any one of SEQ ID NO: 12 or 13. In some embodiments, the antisense sequence is a 16 nucleotide sequence which is at least 90% complementary, or at least 95% complementary, or at least 100% complementary to any one of SEQ ID NO 12 or 13, or a 17 nucleotide sequence which is at least 90% complementary, or at least 95% complementary, or at least 100% complementary to any one of SEQ ID NO 12 or 13, a 18 nucleotide sequence which is at least 90% complementary, or at least 95% complementary, or at least 100% complementary to any one of SEQ ID NO 12 or 13, a 19 nucleotide sequence which is at least 90% complementary, or at least 95% complementary, or at least 100% complementary to any one of SEQ ID NO 12 or 13, or a 20 nucleotide sequence which is at least 90% complementary, or at least 95% complementary, or at least 100% complementary to any one of SEQ ID NO 12 or 13, a 21 nucleotide sequence which is at least 90% complementary, or at least 95% complementary, or at least 100% complementary to any one of SEQ ID NO 12 or 13, or a 22 nucleotide sequence which is at least 90% complementary, or at least 95% complementary, or at least 100% complementary to any one of SEQ ID NO 12 or 13. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 60-102. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 60-102. In some embodiments, the nucleotide sequence may consist of 17, 18, 19, 20, 21, 22, 23, 24, 25, 26, 27 28, 29 or 30 nucleotides.

In some embodiments, the antisense sequence is capable of at least partially binding to an exonic sequence enhancer in the STMN2 cryptic exon, defined by ESE finder 3.0, i.e., and flanking regions thereof, preferably wherein the antisense sequence is at least 90% complementary, or at least 95% complementary, or at least 100% complementary to any one of SEQ ID NO: 14, 15, 16, 17, or 18. In some embodiments, the antisense sequence is a 16 nucleotide sequence which is at least 90% complementary, or at least 95% complementary, or at least 100% complementary to any one of SEQ ID NO 14, 15, 16, 17, or 18, or a 17 nucleotide sequence which is at least 90% complementary, or at least 95% complementary, or at least 100% complementary to any one of SEQ ID NO 14, 15, 16, 17, or 18, a 18 nucleotide sequence which is at least 90% complementary, or at least 95% complementary, or at least 100% complementary to any one of SEQ ID NO 14, 15, 16, 17, or 18, a 19 nucleotide sequence which is at least 90% complementary, or at least 95% complementary, or at least 100% complementary to any one of SEQ ID NO 14, 15, 16, 17, or 18, or a 20 nucleotide sequence which is at least 90% complementary, or at least 95% complementary, or at least 100% complementary to any one of SEQ ID NO 14, 15, 16, 17, or 18, a 21 nucleotide sequence which is at least 90% complementary, or at least 95% complementary, or at least 100% complementary to any one of SEQ ID NO 14, 15, 16, 17, or 18, or a 22 nucleotide sequence which is at least 90% complementary, or at least 95% complementary, or at least 100% complementary to any one of SEQ ID NO 14, 15, 16, 17, or 18.

In some embodiments, the exonic sequence enhancer is an SRSF1 binding site and the antisense sequence is capable of binding (i.e., complementary to or overlapping with) the motif CAGAAGA within the STMN2 cryptic exon sequence, preferably wherein the antisense sequence is at least 90% complementary, or at least 95% complementary, or at least 100% complementary to SEQ ID NO: 14. In some embodiments, the exonic sequence enhancer is an SRSF2 binding site and the antisense sequence is capable of binding (i.e., i.e., complementary to or overlapping with) the motif GGCUUGUG within the STMN2 cryptic exon sequence, preferably wherein the antisense sequence is at least 90% complementary, or at least 95% complementary, or at least 100% complementary to SEQ ID NO: 15. In some embodiments, the exonic sequence enhancer is an SRSF5 binding site and the antisense sequence is capable of binding (i.e., complementary to or overlapping with) the motif UGACAAG within the STMN2 cryptic exon sequence, preferably wherein the antisense sequence is at least 90% complementary, or at least 95% complementary, or at least 100% complementary to SEQ ID NO: 16. In some embodiments, the exonic sequence enhancer is an SRSF6 binding site and the antisense sequence is capable of binding (i.e., complementary to) the motif UGCGGC within the STMN2 cryptic exon sequence, preferably wherein the antisense sequence is at least 90% complementary, or at least 95% complementary, or at least 100% complementary to SEQ ID NO: 15. In some embodiments, the antisense sequence is capable of binding (i.e., complementary to or overlapping with) both the SRSF6 binding site (UGCGGC) and the SRSF2 binding site (GGCUUGUG) in the STMN2 cryptic exon, preferably wherein the antisense sequence is at least 90% complementary, or at least 95% complementary, or at least 100% complementary to SEQ ID NO: 15

In some embodiments, the antisense sequence comprises a sequence that is at least 80% identical, or at least 85% identical, or at least 90% identical, or at least 95% identical or at least 100% identical to any one of SEQ ID NO 380, 382, 384, 386, 388, 390, 392, 394, 396, 398, 400 or a 402, for a sequence with the same number of nucleotides.

### INSR

In some embodiments, the TDP-43 regulated cryptic exon is an INSR cryptic exon. The TDP-43 regulated INSR cryptic exon is between exon 6 and 7 in the human INSR gene. In some embodiments, the antisense sequence is at least 90% complementary (or at least 95%, or at least 100% complementary) to SEQ ID NO: 9 . . . . In some embodiments, the antisense sequence is at least 90% complementary (or at least 95%, or at least 100% complementary) to SEQ ID NO: 10.

In some embodiments, the antisense sequence is capable of binding to the INSR 3′ splice site, (i.e., the INSR splice acceptor site), i.e., and flanking regions thereof, preferably wherein the antisense sequence is at least 90% complementary, or at least 90% complementary, or at least 100% complementary to SEQ ID NO 31. In some embodiments, the antisense sequence is a 16 nucleotide sequence which is at least 90% complementary, or at least 95% complementary, or at least 100% complementary to of SEQ ID NO 31, or a 17 nucleotide sequence which is at least 90% complementary, or at least 95% complementary, or at least 100% complementary to SEQ ID NO 31, a 18 nucleotide sequence which is at least 90% complementary, or at least 95% complementary, or at least 100% complementary to SEQ ID NO 31, a 19 nucleotide sequence which is at least 90% complementary, or at least 95% complementary, or at least 100% complementary to any one of SEQ ID NO 31, or a 20 nucleotide sequence which is at least 90% complementary, or at least 95% complementary, or at least 100% complementary to SEQ ID NO 31, a 21 nucleotide sequence which is at least 90% complementary, or at least 95% complementary, or at least 100% complementary to SEQ ID NO 31, or a 22 nucleotide sequence which is at least 90% complementary, or at least 95% complementary, or at least 100% complementary to SEQ ID NO 31. In some embodiments, the antisense sequence is capable of binding to one of more of the motifs UAUAG/U, AUAG/UA, UAG/UAC, AG/UACC, G/UACCG in the INSR cryptic exon and flanking regions, wherein/represents the intron/cryptic exon boundary of the INSR cryptic exon. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 261-277. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 261-277. In some embodiments, the nucleotide sequence may consist of 17, 18, 19, 20, 21, 22, 23, 24, 25, 26, 27 28, 29 or 30 nucleotides.

In some embodiments, the antisense sequence is capable of at least partially binding to the TDP-43 binding region, or TDP-43 binding motif, of the INSR cryptic exon, i.e., or flanking regions thereof, preferably wherein the antisense sequence is at least 90% complementary, or at least 95% complementary, or at least 100% complementary to any one of SEQ ID NO: 32 or 33. In some embodiments, the antisense sequence is a 16 nucleotide sequence which is at least 90% complementary, or at least 95% complementary, or at least 100% complementary to any one of SEQ ID NO 32 or 33, or a 17 nucleotide sequence which is at least 90% complementary, or at least 95% complementary, or at least 100% complementary to any one of SEQ ID NO 32 or 33, a 18 nucleotide sequence which is at least 90% complementary, or at least 95% complementary, or at least 100% complementary to any one of SEQ ID NO 32 or 33, a 19 nucleotide sequence which is at least 90% complementary, or at least 95% complementary, or at least 100% complementary to any one of SEQ ID NO 32 or 33, or a 20 nucleotide sequence which is at least 90% complementary, or at least 95% complementary, or at least 100% complementary to any one of SEQ ID NO 32 or 33, a 21 nucleotide sequence which is at least 90% complementary, or at least 95% complementary, or at least 100% complementary to any one of SEQ ID NO 32 or 33, or a 22 nucleotide sequence which is at least 90% complementary, or at least 95% complementary, or at least 100% complementary to any one of SEQ ID NO 32 or 33.

In some embodiments, the antisense sequence is capable of at least partially binding to an exonic sequence enhancer in the INSR cryptic exon, defined by ESE finder 3.0, preferably wherein the antisense sequence is at least 90% complementary, or at least 95% complementary, or at least 100% complementary to any one of SEQ ID NO: 34, 35, 36, 37, 38, 39 or 40. In some embodiments, the antisense sequence is a 16 nucleotide sequence which is at least 90% complementary, or at least 95% complementary, or at least 100% complementary to any one of SEQ ID NO 34, 35, 36, 37, 38, 39 or 40, or a 17 nucleotide sequence which is at least 90% complementary, or at least 95% complementary, or at least 100% complementary to any one of SEQ ID NO 34, 35, 36, 37, 38, 39 or 40, a 18 nucleotide sequence which is at least 90% complementary, or at least 95% complementary, or at least 100% complementary to any one of SEQ ID NO 34, 35, 36, 37, 38, 39 or 40, a 19 nucleotide sequence which is at least 90% complementary, or at least 95% complementary, or at least 100% complementary to any one of SEQ ID NO 34, 35, 36, 37, 38, 39 or 40, or a 20 nucleotide sequence which is at least 90% complementary, or at least 95% complementary, or at least 100% complementary to any one of SEQ ID NO 34, 35, 36, 37, 38, 39 or 40, a 21 nucleotide sequence which is at least 90% complementary, or at least 95% complementary, or at least 100% complementary to any one of SEQ ID NO 34, 35, 36, 37, 38, 39 or 40, or a 22 nucleotide sequence which is at least 90% complementary, or at least 95% complementary, or at least 100% complementary to any one of SEQ ID NO 34, 35, 36, 37, 38, 39 or 40. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 278-352. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 278-352. In some embodiments, the nucleotide sequence may consist of 17, 18, 19, 20, 21, 22, 23, 24, 25, 26, 27 28, 29 or 30 nucleotides.

In some embodiments, the exonic sequence enhancer is an SRSF1 binding site and the antisense sequence is capable of binding (i.e., complementary to or overlapping with) the motif GACACCT or CTGAAGA within the INSR cryptic exon, preferably wherein the antisense sequence is at least 90% complementary, or at least 95% complementary, or at least 100% complementary to any one of SEQ ID NO: 34 or 35. In some embodiments, the exonic sequence enhancer is an SRSF2 binding site and the antisense sequence is capable of binding (i.e., i.e., complementary to or overlapping with) the motif GAAUGAUG or GGCUGAUG within the INSR cryptic exon sequence, preferably wherein the antisense sequence is at least 90% complementary, or at least 95% complementary, or at least 100% complementary to any one of SEQ ID NO: 36 or 37. In some embodiments, the exonic sequence enhancer is an SRSF5 binding site and the antisense sequence is capable of binding (i.e., complementary to or overlapping with) the motif AUACAAG within the INSR cryptic exon sequence, preferably wherein the antisense sequence is at least 90% complementary, or at least 95% complementary, or at least 100% complementary to SEQ ID NO: 38. In some embodiments, the exonic sequence enhancer is an SRSF6 binding site and the antisense sequence is capable of binding (i.e., complementary to) the motif UACGGG or UGUGUA within the INSR cryptic exon sequence, preferably wherein the antisense sequence is at least 90% complementary, or at least 95% complementary, or at least 100% complementary to any one of SEQ ID NO: 39 or 40.

### ELAVL3

In some embodiments, the TDP-43 regulated cryptic exon is an ELAVL3 cryptic exon. In some embodiments, the antisense sequence is at least 90% complementary (or at least 95%, or at least 100% complementary) to SEQ ID NO: 431.

In some embodiments, the antisense sequence is capable of binding to the ELALV3 3′ splice site, (i.e., the ELAVL3 splice acceptor site), i.e., and flanking regions thereof, preferably wherein the antisense sequence is at least 90% complementary, or at least 90% complementary, or at least 100% complementary to SEQ ID NO 437.

In some embodiments, the antisense sequence is capable of binding to a ELAVL3 TDP-43 binding region i.e., and flanking regions thereof, preferably wherein the antisense sequence is at least 90% complementary, or at least 90% complementary, or at least 100% complementary to SEQ ID NO 438, 439 or 440.

### G3BP1

In some embodiments, the TDP-43 regulated cryptic exon is an G3BP1 cryptic exon. In some embodiments, the antisense sequence is at least 90% complementary (or at least 95%, or at least 100% complementary) to SEQ ID NO: 432.

In some embodiments, the antisense sequence is capable of binding to the G3BP13 3′ splice site, (i.e., the G3BP1 splice acceptor site), i.e., and flanking regions thereof, preferably wherein the antisense sequence is at least 90% complementary, or at least 90% complementary, or at least 100% complementary to SEQ ID NO 441.

In some embodiments, the antisense sequence is capable of binding to a G3BP1 TDP-43 binding region i.e., and flanking regions thereof, preferably wherein the antisense sequence is at least 90% complementary, or at least 90% complementary, or at least 100% complementary to SEQ ID NO 442

### AARS1

In some embodiments, the TDP-43 regulated cryptic exon is an AARS1 cryptic exon. In some embodiments, the antisense sequence is at least 90% complementary (or at least 95%, or at least 100% complementary) to SEQ ID NO: 433.

In some embodiments, the antisense sequence is capable of binding to the AARS1 3′ splice site, (i.e., the AARS1 splice acceptor site), i.e., and flanking regions thereof, preferably wherein the antisense sequence is at least 90% complementary, or at least 90% complementary, or at least 100% complementary to SEQ ID NO 443.

In some embodiments, the antisense sequence is capable of binding to a AARS1 TDP-43 binding region i.e., and flanking regions thereof, preferably wherein the antisense sequence is at least 90% complementary, or at least 90% complementary, or at least 100% complementary to SEQ ID NO 444 or 445.

### CELF5

In some embodiments, the TDP-43 regulated cryptic exon is an CELF5 cryptic exon. In some embodiments, the antisense sequence is at least 90% complementary (or at least 95%, or at least 100% complementary) to SEQ ID NO: 434.

In some embodiments, the antisense sequence is capable of binding to the CELF5 5′ splice site, (i.e., the CELF5 splice donor site), i.e., and flanking regions thereof, preferably wherein the antisense sequence is at least 90% complementary, or at least 90% complementary, or at least 100% complementary to SEQ ID NO 446.

In some embodiments, the antisense sequence is capable of binding to a CELF5 TDP-43 binding region i.e., and flanking regions thereof, preferably wherein the antisense sequence is at least 90% complementary, or at least 90% complementary, or at least 100% complementary to SEQ ID NO 447, 448 or 449.

### CAMK2B

In some embodiments, the TDP-43 regulated cryptic exon is an CAMK2B cryptic exon. In some embodiments, the antisense sequence is at least 90% complementary (or at least 95%, or at least 100% complementary) to SEQ ID NO: 435.

In some embodiments, the antisense sequence is capable of binding to the CAMK2B 5′ splice site, (i.e., the CAMK2B splice donor site), i.e., and flanking regions thereof, preferably wherein the antisense sequence is at least 90% complementary, or at least 90% complementary, or at least 100% complementary to SEQ ID NO 450.

In some embodiments, the antisense sequence is capable of binding to a CAMK2B TDP-43 binding region i.e., and flanking regions thereof, preferably wherein the antisense sequence is at least 90% complementary, or at least 90% complementary, or at least 100% complementary to SEQ ID NO 451.

### UNC13B

In some embodiments, the TDP-43 regulated cryptic exon is an UNC13B cryptic exon. In some embodiments, the antisense sequence is at least 90% complementary (or at least 95%, or at least 100% complementary) to SEQ ID NO: 436.

In some embodiments, the antisense sequence is capable of binding to the UNC13B 5′ splice site, (i.e., the UNC13B splice donor site), i.e., and flanking regions thereof, preferably wherein the antisense sequence is at least 90% complementary, or at least 90% complementary, or at least 100% complementary to SEQ ID NO 452.

In some embodiments, the antisense sequence is capable of binding to a UNC13B TDP-43 binding region i.e., and flanking regions thereof, preferably wherein the antisense sequence is at least 90% complementary, or at least 90% complementary, or at least 100% complementary to SEQ ID NO 453 or 454.

### Function of Construct

The construct described herein is capable of modulating splicing of the TDP-43 regulated cryptic exon. In other words, the construct described herein is capable of correcting splicing and/or at least partially preventing inclusion of the TDP-43 regulated cryptic exon in mature RNA, such that a functional protein is produced.

In some embodiments, the construct described herein completely rescue splicing (i.e., defined as 0% cryptic exon present in the mature mRNA product of the cell).

In some embodiments, the construct described herein partially rescue splicing i.e., defined as less than 90% cryptic exon present in mature mRNA products of the cell, or less than 80%, or less than 70%, or less than 60%, or less than 50%, or less than 40%, or less than 30%, or less than 20%, or less than 10%, or less than 5% present in mature mRNA products of the cell. This may be determined by RNA-sequencing, RT-qPCR, or RT-PCR. Even partial rescue of correct splicing has some therapeutic benefit and can be used to further understand the role of cryptic exons in TDP-43 pathology.

As defined herein, the cell may be any suitable cell. In preferred embodiments, the cell is a mammalian cell, more preferably a human cell. In preferred embodiments, the cell has nuclear depletion of TDP-43. In some embodiments, the cell is a brain cell. In some embodiments, the cell is a neuron or neuronal cell. In some embodiments, the cell is a microglial cell or astrocyte cell. In some embodiments, the cell is a muscle cell.

### Vector

Also described herein is a vector comprising the modified U7 snRNA construct described herein, or encoding for the modified U7 snRNA construct described herein. The vector encoding for the modified U7 snRNA construct described herein comprises a sequence that is the reverse complement of any modified U7 snRNA construct described herein. The vector typically comprises a promoter upstream of the modified U7 snRNA construct sequence, or upstream of the sequence encoding for the construct, wherein the promoter is a UsnRNA promoter. In some examples described herein, the vector additionally comprises one more expression cassette consisting of a CMV promoter, a blasticidin S deaminase cDNA and an SV40 polyadenylation signal downstream of the U7 snRNA expression cassette. This allows for the selection of cells that have taken up the vector.

The vectors described herein also comprise a 3′ box sequence downstream of the construct sequence or sequence encoding for the construct sequence. Any suitable 3′ box sequence may be used. In some examples, the vectors comprise a 3′ box sequence that has at least 80% sequence identity, or at least 85%, or at least 90%, or at least 95%, or at least 100% sequence identity to SEQ ID NO: 357

In some embodiments, the vector comprises an expression cassette, e.g., an inverted terminal repeat (ITR) cassette. In such systems the vector comprises a sequence encoding for the construct described herein and one or more inverted terminal repeat (ITR) sequences flanking the construct sequence. In some embodiments, the vector is a viral vector. The viral vector may be a human viral vector or a non-human viral vector (e.g., a primate vector). In some embodiments, the vector is a viral vector, such as an adeno-associated (AAV) vector, a retrovirus vector, a lentivirus vector or an adenovirus vector. The viral vector may be an RNA vector or a DNA vector.

### Pharmaceutical Composition

Also disclosed herein is a pharmaceutical composition comprising one or more of the modified U7 snRNA constructs disclosed herein and/or one or more of the vectors disclosed herein. In some embodiments, the pharmaceutical composition comprises two or more, modified U7 snRNA constructs or vectors as defined herein. The two or more constructs or vectors may be capable of binding to the same or different TDP-43 regulated cryptic exons. The two or more constructs or vectors may comprise different antisense sequences. In some embodiments, the different antisense sequences may be capable of binding different splicing elements. The pharmaceutical composition may further comprise a pharmaceutical excipient.

### Therapy and Medicaments

The modified U7 snRNA constructs of the present disclosure, the vectors of the present disclosure, or the pharmaceutical compositions of the present disclosure may be for use in therapy (i.e., as therapeutic agents for disease treatment). The therapeutic use of the constructs of the present disclosure may involve in modulation of splicing of endogenously existing pre-RNAs to at least partially prevent inclusion of a TDP-43 regulated cryptic exon in the mature RNA of the cell transcript. This provides protection from the disease since the absence of the TDP-43 regulated cryptic exon in the mature RNA transcript leads to the production of fully functional protein.

The modified U7 snRNA constructs of the present disclosure, the vectors of the present disclosure, or the pharmaceutical compositions of the present disclosure may be for use, or used, as a medicament, for example, in therapy.

The modified U7 snRNA constructs of the present disclosure, the vectors of the present disclosure, or the pharmaceutical compositions of the present disclosure may be for use in the treatment of a disease associated with TDP-43 pathology or dysfunction. In some embodiments, the disease is a neurodegenerative disease or a neuromuscular disease. In an embodiment, the neurodegenerative disorder is associated with reduced nuclear TDP-43. In an embodiment, the neurodegenerative disorder is caused by nucleus-cytoplasmic mislocalization of TDP-43. In an embodiment, the neurodegenerative disorder is associated with TDP-43 pathology (e.g., pathological TDP-43).

In an embodiment, the construct, vector, or pharmaceutical composition for use or the method of treating comprises first diagnosing a subject with a neurodegenerative disorder associated with TDP-43 pathology. In an embodiment, this is determined using a biomarker of TDP-43 pathology. In an embodiment, this may be determined by genetics, for example, a genetic mutation. In an embodiment, TDP-43 pathology associated with ALS may be determined if FUS and SOD1 mutations are not found in the subject. In an embodiment, TDP-pathology associated with FTD may be determined if C9orf72 or PGRN mutations are not found in the subject. In an embodiment, the biomarker of TDP-43 pathology may include mutant TDP-43. In some embodiments, TDP-43 pathology may be determined with TDP-43 phosphorylation. In some embodiments, TDP-43 pathology may be determined by expression of the STMN2 cryptic exon, which may be determined by RNA-seq. In an embodiment, the construct, vector, or pharmaceutical composition for use or the method of treating comprises first identifying in a subject whether they possess a SNP variant associated with rs12973192 and/or rs12608932 ahead of the method of treating. This may be determined by genomics.

In an embodiment, the disorder (i.e., neurodegenerative disorder) may be selected from ALS, frontotemporal dementia, Alzheimer's disease, disease

Inclusion body myositis/myopathy (IBM), FOSMNN (Facial onset sensory and motor neuronopathy), Perry Syndrome, Limbic-Predominant Age-Related TDP-43 Encephalopathy (LATE) or a combination thereof.

In an embodiment, the neurodegenerative disorder is ALS (amyotrophic lateral sclerosis). ALS is a chronic and fatal form of motor neuron disease (MND) and may otherwise be referred to as MND, Charcot disease or Lou Gehrig's disease. In some embodiments, the ALS may be ALS is familial ALS or sporadic (idiopathic) ALS. Familial ALS (FALS) is ALS that runs in the family, and accounts for about 10% of ALS cases. Sporadic ALS is non-familial ALS. In an embodiment, the ALS may not be an ALS-FUS and ALS-SOD1 which are genetically-defined forms of ALS. The construct, vectors, or pharmaceutical compositions for use, or the method of treatment described herein, may ameliorate one or more symptoms associated with ALS. Symptoms of ALS may include fasciculation (muscle twitches); muscle cramps; tight and stiff muscles (spasticity), muscle weakness, slurred and nasal speech and a difficulty chewing or swallowing. ALS leads to progressive deterioration of muscle function and ultimately often leads to death due to respiratory failure. In an embodiment, the TDP-43 regulated cryptic exon is a UNC13A TDP-43 regulated cryptic exon and the neurodegenerative disorder is ALS. In another embodiment, the TDP-43 regulated cryptic exon is STMN2 TDP-43 regulated cryptic exon and the neurodegenerative disorder is ALS.

In an embodiment, the neurodegenerative disorder is frontotemporal dementia (FTD). Frontotemporal dementia is a type of dementia that affects the frontal and temporal lobes of the brain. The constructs, vectors, or pharmaceutical composition for use, or the method of treatment described herein, may ameliorate one or more symptoms associated with FTD. Symptoms of FTD may include personality and behavior changes, language problems, problems with mental abilities, memory problems and physical problems (e.g., difficulties with movement). The FTD may be characterized by frontotemporal lobar degeneration (FTLD). The FTLD may be FTLD-TDP, which is an FTLD associated with TDP-43 pathology. This may be characterized by ubiquitin and TDP-43 positive, tau negative, FUS negative inclusion bodies. The FTLD-TDP may be of Type A, Type B, Type C or Type D. Type A is a type of FTLD-TDP that presents with small neurites and neuronal cytoplasmic inclusion bodies in the upper (superficial) cortical layers. Bar-like neuronal intranuclear inclusions may also be seen, although comparatively fewer in number. Type B is a type of FTLD-TDP that presents with neuronal and glial cytoplasmic inclusions in both the upper (superficial) and lower (deep) cortical layers, and lower motor neurons. Neuronal intranuclear inclusions may be absent or are in comparatively small number. Type B may be associated with ALS and C9ORF92 mutations. Type C is a type of FTLD-TDP that presents long neuritic profiles found in the superficial cortical laminae. There may be comparatively few or no neuronal cytoplasmic inclusions, neuronal intranuclear inclusions or glial cytoplasmic inclusions. FTLD-TDP is often associated with semantic dementia. Type D is a type of FTLD-TDP that presents with neuronal intranuclear inclusion and dystrophic neurites. There may be no inclusions in the granule cell layer of the hippocampus. Type D may be associated with VCP mutations. In an embodiment, the FTLD may not be of type FTLD-FUS or FTLD-tau. In an embodiment, the TDP-43 regulated cryptic exon is a UNC13A TDP-43 regulated cryptic exon and the neurodegenerative disorder is FTD. In another embodiment, the TDP-43 regulated cryptic exon is STMN2 TDP-43 regulated cryptic exon and the neurodegenerative disorder is FTD.

Also disclosed herein is a method of treating a disease associated with TDP-43 dysfunction (e.g., a neurodegenerative disorder or a muscular disorder) the method comprising administering to a subject in need thereof a therapeutically effective amount of the construct, vector, or pharmaceutical composition disclosed herein.

The construct, vector or pharmaceutical composition described for use or in the methods of treatment herein can be used to prevent loss of and/or restore functionality of certain proteins that are regulated by TDP-43 splicing. In some embodiments, the construct, vector or pharmaceutical composition described for use or in the methods of treatment herein can be used to prevent loss of and/or restore functionality of genes containing a TDP-43 regulated cryptic exon. This may be any gene described herein and includes, for example, UNC13A, STMN2 or INSR.

The construct, vector or pharmaceutical composition for use, or when used as a medicament or used in a method of treatment as described herein may be administered to any suitable subject. In a preferred embodiment, the subject is human. In an embodiment, the subject possesses a SNP variant associated with rs12973192 and/or rs12608932. The human subject is any suitable age, for example, an infant (less than 1 year of age) a child (younger than 18 years of age) including adolescents (10 to 18 years of age inclusive), or adults (older than 18 years of age) including elderly subjects (older than 65 years of age).

The construct, vector or pharmaceutical composition for use, or when used as a medicament or used in a method of treatment as described herein may be administered using any suitable mode of administration.

### Methods of Use

The present disclosure provides methods for use of the constructs, vectors and pharmaceutical compositions of the present disclosure. These methods may be in vivo or in vitro methods.

the constructs, vectors and pharmaceutical compositions re may be used for regulating gene expression at multiple levels. Some aspects of the present disclosure provide methods for regulation gene expression in a cell comprising administering to the cell the construct, vector or pharmaceutical compositions described herein. In some embodiments, the gene expression is regulated at the transcription level, or post-transcription level, or translational level, or post-translational level.

Disclosed herein is a method of modulating splicing of a TDP-43 regulated cryptic exon, the method comprising delivering to a cell the construct described herein, the vector of described herein, or the pharmaceutical composition of described herein, wherein the method comprises contacting the construct with a cell to modulate splicing of the TDP-43 regulated cryptic exon.

Disclosed herein is a method of modulating splicing of the UNC13A cryptic exon, the method comprising delivering to a cell a construct described herein, the vector of described herein, or the pharmaceutical composition of described herein, each comprising an antisense sequence that is at least 90% complementary with SEQ ID NO: 1 or 2 wherein the method comprises contacting the construct with a cell to modulate splicing of the UNC13A cryptic exon. In some embodiments, the antisense sequence is at least 90% complementary with SEQ ID NO: 3 or 4.

Disclosed herein is a method of modulating splicing of the STMN2 cryptic exon 2a, the method comprising delivering to a cell a construct described herein, the vector of described herein, or the pharmaceutical composition of described herein, each comprising an antisense sequence that is at least 90% complementary with SEQ ID NO: 7, wherein the method comprises contacting the construct with a cell to modulate splicing of the STMN2 cryptic exon 2a.

Disclosed herein is a method of modulating splicing of the INSR cryptic exon, the method comprising delivering to a cell a construct described herein, the vector of described herein, or the pharmaceutical composition of described herein, each comprising an antisense sequence that is at least 90% complementary with SEQ ID NO: 9, wherein the method comprises contacting the construct with a cell to modulate splicing of the INSR cryptic exon 2a.

Disclosed herein is a method of preventing inclusion of the TDP-43 regulated cryptic exon in the mature mRNA of a cell transcript, the method comprising delivering to a cell the construct described herein, the vector of described herein, or the pharmaceutical composition of described herein, wherein the method comprises contacting the construct with a cell to prevent inclusion of the TDP-43 regulated cryptic exon in the mature mRNA of a cell transcript.

### Combined Vector

According to a seventh aspect of the present invention, is a combined vector comprising two or more of the constructs described herein or of the first aspect (i.e., in tandem, or one downstream of another, such that the construct comprises more than one antisense sequence as defined herein). In preferred embodiments, the two or more modified U7 snRNA constructs comprise different antisense sequences that are capable of binding to (i.e., at least 90%, or at least 95%, or 100% complementary to) different TDP-43 regulated cryptic exons described herein. For example, the combined vector may comprise a first construct comprising an antisense sequence which is at least 90% complementary to (or at least 95%, or 100% complementary to) a first TDP-43 regulated cryptic exon, and a second construct comprising an antisense sequence which is at least 90% complementary to (or at least 95%, or at least 100% complementary to) a second TDP-43 regulated cryptic exon, wherein the second TDP-43 regulated cryptic exon is different from the first. In some embodiments, the combined vector may comprise three or more constructs as defined herein. The TDP-43 regulated cryptic exon may be any TDP-43 regulated cryptic exon as described herein. In some embodiments, the antisense sequence(s) are sequence(s) which are at least 90% complementary (or at least 95%, or 100% complementary) to SEQ ID NO: 1, 2, 3, 4, 7, 9 or 431-436). In some embodiments, at least one of the antisense sequences, or each antisense sequences, is complementary to a TDP-43 binding region of the TDP-43 regulated cryptic exon, preferably wherein at least one of the antisense sequences, or each antisense sequence, is complementary (i.e., 90%, 95% or 100% complementary) to SEQ ID NO: 12, 23-26 or 32. In some embodiments, the combined vector comprises a construct as defined herein comprising an antisense sequence which is at least 90% (or at least 95%, or 100%) complementary to a UNC13A TDP-43 regulated cryptic exon or flanking region thereof and a construct as defined herein comprising an antisense sequence which is at least 90% (or at least 95%, or 100%) complementary to a STMN2 TDP-43 regulated cryptic exon or flanking region thereof. In some embodiments, the combined vector comprises a construct as defined herein comprising an antisense sequence which is at least 90% (or at least 95%, or 100%) complementary to UNC13A TDP-43 regulated cryptic exon or flanking region thereof and a construct as defined herein comprising an antisense sequence which is at least 90% (or at least 95%, or 100%) complementary to a INSR TDP-43 regulated cryptic exon or flanking region thereof. In some embodiments, the combined vector comprises a construct as defined herein comprising an antisense sequence which is at least 90% (or at least 95%, or 100%) complementary to a STMN2 TDP-43 regulated cryptic exon or flanking region thereof and a construct as defined herein comprising an antisense sequence which is at least 90% (or at least 95%, or 100%) complementary to a INSR TDP-43 regulated cryptic exon or flanking region thereof. In some embodiments, the combined vector comprises a construct comprising an antisense sequence which is at least 90% (or at least 95%, or 100%) complementary to a UNC13A TDP-43 regulated cryptic exon or flanking region thereof, a construct comprising an antisense sequence which is at least 90% (or at least 95%, or 100%) complementary to a STMN2 TDP-43 regulated cryptic exon or flanking region thereof, and a construct comprising an antisense sequence which is at least 90% (or at least 95%, or 100%) complementary to a INSR TDP-43 regulated cryptic exon or flanking region thereof. In some embodiments, at least one of the two or more constructs in the combined vector further comprises a binding domain sequence for a hnRNP protein, for example, for a hnRNP A or hnRNP H protein. In some embodiments, the combined vector comprises more than one binding domain sequence for a hnRNP protein. In some embodiments, each construct (i.e., constituent construct) comprises a binding domain sequence for a hnRNP proteins such that the combined vector comprises the same number of binding domain sequences for hnRNP proteins as there are antisense sequences.

In some embodiments, the combined vector comprises at least one construct, wherein the construct further comprises a binding domain sequence for a hnRNP protein, for example, a hnRNP A or hnRNP H protein. In some embodiments, each construct (i.e., constituent construct) comprises a binding domain sequence for a hnRNP proteins (i.e., such that the combined vector comprises the same number of binding domain sequences for hnRNP proteins as there are antisense sequences). The hnRNP binding domain serves to recruit endogenous hnRNP protein which helps take over the repressive function that TDP-43 normally has in “healthy” cells. The constructs described herein comprise a sequence comprising a binding domain for a hnRNP protein. The term binding domain may be used interchangeably with hnRNP binding site, hnRNP binding sequence or hnRNP binding domain. The sequence comprising a binding domain for a hnRNP protein may also be described as a hnRNP tail herein. A hnRNP protein defined herein and as known in the art is a heterogeneous nuclear ribonucleoprotein. These are a family of RNA-binding proteins that participate in pre-mRNA processing. The definition of a hnRNP protein described herein is not intended to include or encompass the protein TDP-43. In preferred embodiments, the hnRNP protein is a hnRNP protein comprising at least 2 RNA recognition motifs or quasi-RNA recognition motifs. In preferred embodiments, the hnRNP protein is a protein that is highly endogenously expressed in a human cell, more particularly a human cell nucleus. In some embodiments, highly endogenous expressed refers to any protein with a “high” protein expression or a “high” protein expression score in neuronal and/or glial cells in any part of the brain as defined by human protein atlas. (i.e., https://www.proteinatlas.org/). In some embodiments, the part of the brain may be selected from the basal ganglia, hippocampus, cerebellum, cerebral cortex, or a combination thereof. In some embodiments, the protein expression score in any part of the brain may be at least 100, or at least 150, or at least 200, or at least 250, or at least 300, or at least 350 nTPM in the brain, which may be determined in accordance with the consensus data set on human protein atlas, wherein nTPM refers to normalised transcript expression values per million. In some embodiments, the hnRNP protein is not a hnRNP protein that has to form a tetramer (e.g., hnRNP C) and/or a hnRNP that functions by binding on both sides of the cryptic exon in order to have a repressive effect on splicing. In some embodiments, the hnRNP protein is selected from a hnRNP A and hnRNP H protein, and the sequence comprising a binding domain comprises at least one binding motif for hnRNP A and hnRNP H respectively. These are found to more effectively correct splicing compared to other hnRNP proteins tested (e.g., hnRNP C or hnRNP L). In some embodiments, the hnRNP A protein is hnRNP A1 or hnRNP A2 . . . . In some examples, the hnRNP A protein in hnRNP A1. hnRNP A1 and hnRNP A2 are the most abundant hnRNPs with nearly identical functions, and play important roles in regulating gene expression at multiple levels. In some embodiments, the sequence comprising a binding domain for a hnRNP protein is from about 8 to 24 nucleotides, preferably from about 16 nucleotides to 22 nucleotides, or about 20 nucleotides. In some embodiments, the binding sequence for a hnRNP protein comprises at least 8, preferably at least 16 nucleotides, or at least 17 nucleotides (or 17 nucleotides), or at least 18 nucleotides (or 18 nucleotides), or at least 19 nucleotides (or 19 nucleotides), or at least 20 nucleotides (or 20 nucleotides). In some embodiments, the binding sequence comprises one, two or three binding motifs for a hnRNP protein. Example binding motifs are described below. In some embodiments, the binding sequence is a binding sequence for hnRNP A (e.g. hnRNP A1 or hnRNP A2). The binding sequence for hnRNP A may be any sequence or comprise any motif known in the art to bind hnRNP A (e.g. hnRNP A1 or hnRNP A2). In some embodiments the binding sequence for hnRNP A (e.g. hnRNP A1 or hnRNP A2) may have been determined by immunoprecipitation of hnRNP A1 to the human transcriptome, e.g., using CLIP. In some embodiments, the binding sequence for a hnRNP A protein (e.g., hnRNP A1 or hnRNP A2) comprises at least one or two motifs comprising UAGGG. In some embodiments, the binding sequence for hnRNP A1 comprises at least one motif according to WUAGGGWS, where Wis A or U, and wherein S is C or G, and preferably wherein the binding sequence for hnRNP A1 comprises at least two motifs according to WUAGGGWS, where W is A or U, and wherein S is C or G. In some embodiments, the binding sequence for hnRNP A1 comprises at least one or two motifs selected from UAGGG (more preferably UUAGGG, more preferably UAGGGU or UAGGGA, and furthermore preferably UUAGGGUG), or ATAGGGA (more preferably ATAGGGAC). In some embodiments, the binding sequence is at least 80% identical, or at least 85% identical, or at least 90% identical, or at least 100% identical to UAUGAUAGGGACUUAGGGUG (SEQ ID NO: 455) or UAUGUUAGGGACUUAGGGAG (SEQ ID NO: 456). In some embodiments, the binding sequence for hnRNP A2 comprises at last one or two motifs selected from UAGGG, GGUAGUAG, or AGGAUAGA. In some embodiments, the binding sequence is a binding sequence for hnRNP H. hnRNP H may encompass both hnRNP H1 and hnRNP H2. The binding sequence for hnRNP H may be any sequence or motif known in the art to bind hnRNP H. In some embodiments the binding sequence for hnRNP H may have been determined by immunoprecipitation of hnRNP H to the human transcriptome, e.g., using CLIP. In some embodiments, the binding sequence for hnRNP H comprises one or two binding motifs comprising the motif GGGGA. In some embodiments, the binding sequence is at least 80% identical, or at least 85% identical, or at least 90% identical, or at least 100% identical to UGUGGGGACCUUGUGGGGACC (SEQ ID NO: 457) or UGUGGGGACCAGGGGGA (SEQ ID NO: 458).

In some embodiments, the combined vector comprises two or more promoter sequences, wherein the two or more promoter sequences are upstream of each construct. The promoters may be any promoter sequence used in the art. In some embodiments, each of the two or more promoter sequences are the same. In some embodiments, the combined vector comprises two or more 3′ box sequences, wherein the two or more 3′ box sequences are downstream of each construct. The 3′ box sequences may be the same or different and may be any 3′ box sequence used in the art.

In some embodiments, the combined vector comprises two or more U7 cassettes, wherein each cassette comprises a promoter, a modified U7 snRNA construct as defined herein, and a 3′ box sequence, wherein the promoter is upstream of the modified U7 snRNA construct and the 3′ box sequence is downstream of the modified U7 snRNA construct. In some embodiments, the combined vector comprises a stuffer sequence between each of the two or more U7 cassettes. The stuffer sequences serve to space out the at least two promoters. The stuffer sequence may be any suitable stuffer sequence used in the art.

### EXAMPLES

The modified U7 snRNA constructs described in the Examples are all U7 smOPT constructs designed to target TDP-43 regulated cryptic exon sequences to restore correct splicing in TDP-43 depleted cells. The U7 smOPT constructs comprise (i) an antisense sequence designed to target a TDP-43 regulated cryptic exon and flanking regions thereof, and (ii) a modified Sm sequence (smOPT sequence).

### UNC13A

One such TDP-43 regulated cryptic exon is in the gene UNC13A, which is located between exons 20 and 21. SEQ ID NO 1-shows a portion of UNC13A transcribed pre mRNA intronic sequence including the cryptic exon sequence and flanking regions thereof, including the TDP-43 binding region in the proximity of the cryptic exon as determined by iCliP. The shorter cryptic exon sequence is in italics and the longer cryptic exon sequence is underlined. The lower-case bases denote the bases immediately flanking the splice donor site (gu) and the splice acceptor sites (ag). The ESE targets identified by ESE finder 3.0 are shown in bold.

UGGGAAGCCCACCUUGGCCUCCAGGUUGACUCUCACUACUCAUCAUCAG
GUUCUUCCUUCUAUUCCagCCCUAACCACUCAGGAUUGGGCCGUUUGUG
UCUGGGUAUGUCUCUUCCagCUGCCUGGGUUUCCUGGAAAGAACUCUUA
UCCCCAGGAACUAGUUUGUUGAAUAAAUGCUGGUGAAUGAAUGAAUGAU
UGAACAGAUGAAUGAGUGAUGAGUAGAUAAAAGGAUGGAUGGAGAGAUG
GguGAGUACAUGGAUGGAUAGAUGGAUGAGUUGGUGGGUAGAUUCGUGG
CUAGAUGGAUGAUGGAUGGAUGGACAGAUGGAUGGAUAUAUGAUUGAAC
UAUUGAAAGUAUAGAUGUAUGGAUGGGUGAAUUUGGGGGUAAUUGUUAG
AUGAUGGAUGAGUAUAGAUGAAUGAUGGAUGGAUAACUUGAUGAGUGGA
UAGAUAGAUUGCUGGAUAGAUGAUUGACUGGGUGGAUAGAUGAAAUGUU
GGAUGAGCAGAUUAAGUUGUAUUGGAUGGGAUGGAUGGAAGUGUGGUUG
AGUUAUUAGAAGGAAGAUUGAGUAGAUAGGUGAAUUUGUUGAUAGUCAG
AUGGGUAGAUAGGUAGAUGGAUGGAUGGAUGGAUGGAUGUAUAGGCAGA
UGGACAAAUGGAUGAAUGGGUGGGUGGAUGAAUGGAAGGAUGUGUGGUU
GAACUAUUGCAAGUAUUGAUAAUUGGGUUCAUAAUUUCUGAAUAUUUAG
AUGGAUGGUUGUGAGUGGCUGGUGGACAGACGAAAAAUGGAUGGUUGGA
UAAAUUGAUGGGUGGAUGGAUGGUUGGUUGUAUGAAAGAAUGAAUGAUU
GGGUAGGUGGAUUAAGUUGCGGAUCAAUGUAUGGGAUGGAUGAAUGGAU
GGAUGGAUGGAUGUGUGGUUGAAUUACUGAAAGGUUGGAAGAGUGGAUG
GGUGAAAUUUGGGGUAGUUAGAUGGGUGGGUGUGUGGAUGGAUAAAAGA
GUAGAUGAAUG

The ESE targets correspond to binding sites for SR proteins, these motifs are as follows:

SRSF5
(ACUCAGG),
SRSF1
(CUCAGGA),
SRSF6
(UGUGUC)
and
SRSF2
(GUUUCCUG).

SEQ ID NO: 1 is reproduced again below. Here, the SNPs are located at position rs12973192 (i.e., within the UNC13A CE sequence), and rs12608932 (i.e., within the intronic region) are shown underlined and the TDP-43 binding region is shown in bold (i.e., as determined by iCLIP data).

UGGGAAGCCCACCUUGGCCUCCAGGUUGACUCUCACUACUCAUCAUCAG
GUUCUUCCUUCUAUUCCagCCCUAACCACUCAGGAUUGGGCCGUUUGUG
UCUGGGUAUGUCUCUUCCagCUGCCUGGGUUUCCUGGAAAGAACUCUUA
UCCCCAGGAACUAGUUUGUUGAAUAAAUGCUGGUGAAUGAAUGAAUGAU
UGAACAGAUGAAUGAGUGAUGAGUAGAUAAAAGGAUGGAUGGAGAGAUG
GguGAGUACAUGGAUGGAUAGAUGGAUGAGUUGGUGGGUAGAUUCGUGG
CUAGAUGGAUGAUGGAUGGAUGGACAGAUGGAUGGAUAUAUGAUUGAAC
UAUUGAAAGUAUAGAUGUAUGGAUGGGUGAAUUUGGGGGUAAUUGUUAG
AUGAUGGAUGAGUAUAGAUGAAUGAUGGAUGGAUAACUUGAUGAGUGGA
UAGAUAGAUUGCUGGAUAGAUGAUUGACUGGGUGGAUAGAUGAAAUGUU
GGAUGAGCAGAUUAAGUUGUAUUGGAUGGGAUGGAUGGAAGUGUGGUUG
AGUUAUUAGAAGGAAGAUUGAGUAGAUAGGUGAAUUUGUUGAUAGUCAG
AUGGGUAGAUAGGUAGAUGGAUGGAUGGAUGGAUGGAUGUAUAGGCAGA
UGGACAAAUGGAUGAAUGGGUGGGUGGAUGAAUGGAAGGAUGUGUGGUU
GAACUAUUGCAAGUAUUGAUAAUUGGGUUCAUAAUUUCUGAAUAUUUAG
AUGGAUGGUUGUGAGUGGCUGGUGGACAGACGAAAAAUGGAUGGUUGGA
UAAAUUGAUGGGUGGAUGGAUGGUUGGUUGUAUGAAAGAAUGAAUGAUU
GGGUAGGUGGAUUAAGUUGCGGAUCAAUGUAUGGGAUGGAUGAAUGGAU
GGAUGGAUGGAUGUGUGGUUGAAUUACUGAAAGGUUGGAAGAGUGGAUG
GGUGAAAUUUGGGGUAGUUAGAUGGGUGGGUGUGUGGAUGGAUAAAAGA
GUAGAUGAAUG

The splice sites are defined as follows: Long cryptic acceptor is the phosphodiester bond between chr19: 17,642,591-17,642,592; the Short cryptic acceptor is the phosphodiester bond between chr19: 17,642,541-17,642,542 and the Cryptic donor is the phosphodiester bond between chr19: 17,642,413-17,642,414.

SEQ ID NO: 1 encompasses the minor allele of the SNP (i.e., the risk variant) or the major allele at rs12973192 and/or rs12608932, therefore SEQ ID NO: 1 also encompasses the sequence with SNP at these positions (e.g., wherein the G at rs12973192 is replaced with a C, defined by SEQ ID NO: 2).

SEQ ID NO: 3 shows a shorter portion of UNC13A transcribed pre mRNA intronic sequence including the cryptic exon sequence and flanking regions thereof.

UGGGAAGCCCACCUUGGCCUCCAGGUUGACUCUCACUACUCAUCAUCAG
GUUCUUCCUUCUAUUCCagCCCUAACCACUCAGGAUUGGGCCGUUUGUG
UCUGGGUAUGUCUCUUCCagCUGCCUGGGUUUCCUGGAAAGAACUCUUA
UCCCCAGGAACUAGUUUGUUGAAUAAAUGCUGGUGAAUGAAUGAAUGAU
UGAACAGAUGAAUGAGUGAUGAGUAGAUAAAAGGAUGGAUGGAGAGAUG
GguGAGUACAUGGAUGGAUAGAUGGAUGAGUUGGUGGGUAGAUUCGUGG
CUAGAUGGAUGAUGGAUGGAUGGACA

SEQ ID NO: 3 encompasses the minor allele of the SNP (i.e., the risk variant) or the major allele at rs12973192 and/or rs12608932, therefore SEQ ID NO: 3 also encompasses the sequence wherein the G at rs12973192 is replaced with a C. This is defined by SEQ ID NO: 4.

SEQ ID NO: 5 corresponds to the shorter UNC13A cryptic exon sequence transcribed UNC13A mRNA-cords chr19: 17642414-17,642,541.

SEQ ID NO: 5 has the sequence:

CUGCCUGGGUUUCCUGGAAAGAACUCUUAUCCCCAGGAACUAGUUUGUU
GAAUAAAUGCUGGUGAAUGAAUGAAUGAUUGAACAGAUGAAUGAGUGAU
GAGUAGAUAAAAGGAUGGAUGGAGAGAUGG).

SEQ ID NO: 5 encompasses minor allele of the SNP (i.e., the risk variant), or the major allele at rs12973192, therefore SEQ ID NO: 5 also encompasses the sequence wherein the G at rs12973192 is replaced with a C.

SEQ ID NO 6 corresponds to the longer UNC13A cryptic exon sequence in transcribed UNC13A mRNA-cords chr19: 17642414-17642591.

SEQ ID NO 6 has the sequence

CCCUAACCACUCAGGAUUGGGCCGUUUGUGUCUGGGUAUGUCUCUUCCA
GCUGCCUGGGUUUCCUGGAAAGAACUCUUAUCCCCAGGAACUAGUUUGU
UGAAUAAAUGCUGGUGAAUGAAUGAAUGAUUGAACAGAUGAAUGAGUGA
UGAGUAGAUAAAAGGAUGGAUGGAGAGAUGG.

SEQ ID NO: 6 may encompass the risk variant of the SNP (i.e., minor allele), or the major allele at rs12973192, therefore SEQ ID NO: 6 also encompasses the sequence wherein the G at rs12973192 is replaced with a C.

### STMN2

Another TDP-43 regulated cryptic exon is in the gene STMN2, corresponding to exon 2a of the STMN2 gene.

SEQ ID NO 7-shows a portion of STMN2 transcribed pre mRNA intronic sequence and part of the cryptic exon sequence 2a. The lower-case bases denote the bases immediately flanking the splice acceptor site (ag). The polyA site is shown underlined. The ESE targets identified by ESE finder 3.0 are shown in bold. The TDP-43 binding motif is shown underlined.

UGCCCCAUCACUCUCUCUUAAUUGGAUUUUUAAAAUUAUAUUCAUAUUG
CagGACUCGGCAGAAGACCUUCGAGAGAAAGGUAGAAAAUAAGAAUUUG
GCUCUCUGUGUGAGCAUGUGUGCGUGUGUGCGAGAGAGAGAGACAGACA
GCCUGCCUAAGAAGAAAUGAAUGUGAAUGCGGCUUGUGGCACAGUUGAC
AAGGAUGAUAAAUCAAUAAUGCAAGCUUACUAUCAUUUAUGAAUAGCAA
UACUGAAGAAAUUAAAACAAAAGAUUGCUGUCUC

The ESE targets in the STMN2 cryptic exon and flanking regions thereof correspond to binding sites for SRSF1 (CAGAAGA), SRSF2 (GGCUUGUG), SRSF5 (UGACAAG) and SRSF6 (UGCGGC).

SEQ ID NO: 8 shows the STMN2 cryptic exon 2a. This has the genomic position chr8: 79,616,822-79,617,048

GACUCGGCAGAAGACCUUCGAGAGAAAGGUAGAAAAUAAGAAUUUGGCU
CUCUGUGUGAGCAUGUGUGCGUGUGUGCGAGAGAGAGAGACAGACAGCC
UGCCUAAGAAGAAAUGAAUGUGAAUGCGGCUUGUGGCACAGUUGACAAG
GAUGAUAAAUCAAUAAUGCAAGCUUACUAUCAUUUAUGAAUAGCAAUAC
UGAAGAAAUUAAAACAAAAGAUUGCUGUCUC

### INSR

SEQ ID NO: 9 shows a portion of INSR transcribed pre mRNA intronic sequence with cryptic exon corresponding to the genomic position chr19, complement: 7169720-716983. The lower-case bases denote the bases immediately flanking the splice acceptor site (ag). The ESE targets identified by ESE finder 3.0 are shown in bold. The TDP-43 binding motif is shown underlined.

UAUUAAUAUUAUCACUAUGCUUACUGUGCCAUAUagUACCGGAUACGGG
AUGAAGUCAUACAAGCACUGAAUGAAUGGAUGAAUGAAUGAUGGAUGAA
UGGAUGACACCUUCUUAUAUGUGUAUCAGGCUGAUGCUGAAGACUUCAA
AGUUGAGUAAAAUACCUAUGUCAGUC

The ESE targets correspond to binding sites for SRSF1 (GACACCT and CTGAAGA), SRSF2 (GAAUGAUG and GGCUGAUG), SRSF5 (AUACAAG) and SRSF6 (UACGGG and UGUGUA).

SEQ ID NO: 10 shows the INSR cryptic exon

UACCGGAUACGGGAUGAAGUCAUACAAGCACUGAAUGAAUGGAUGAAUG
AAUGAUGGAUGAAUGGAUGACACCUUCUUAUAUGUGUAUCAGGCUGAUG
CUGAAGACUUCAAA

### ELALV3

SEQ ID NO: 431 shows a portion of ELAVL3 transcribed pre mRNA intronic sequence with cryptic exon corresponding to the genomic position chr19, complement: 11463496-11463662 The lower-case bases denote the bases immediately flanking the splice acceptor site (ag). The TDP-43 binding region is shown underlined.

CCCGGCCCAGGAUGACGUGCUUAUUAUUUGACagGUGCAUGUGACACUG
UGACUCCGGCUGUGACCUGAUGGGGCCUCAGGGAUGCGUCUGGCUCUGG
CAGGAUGUUUGUGUGUCACCGCGAUGUUGUGUGGGUGUGUCUACCUGUG
CCCUGCUCUGAGGGAUUGAGUGUGAUAUCGUGUGUUUGUGCUGCGCUGU
GAUGG

### G3BP1

SEQ ID NO: 432 shows a portion of G3BP1 transcribed pre mRNA intronic sequence with cryptic exon corresponding to the genomic position chr5, complement: 151787765-151787794

The lower-case bases denote the bases immediately flanking the splice acceptor site (ag). The TDP-43 bindina region is shown underlined.

GACCAGAACUAUUUUUUCCCUUACACCUUGACCagCUUGCAUAUUGGAU
ACCACAUGAUUAUCAG

### AARS1

SEQ ID NO: 433 shows a portion of AARS1 transcribed pre mRNA intronic sequence with cryptic exon corresponding to the genomic position chr16, complement: 70272796-70272882 The lower-case bases denote the bases immediately flanking the splice acceptor site (ag). The TDP-43 binding region is shown underlined.

AUCUUUUGUGUGUGUGUGUGUGUGUGUGUGUGUGUGUGUGUCACCCagG
CUGGAGUGCAGUGGCAUGAUCACAGCUCACUGCAGCCUCAACUUCCUGG
GCUCAAGUGAUCCUCUCCCGAGUAGCUGGGACUACAG

### CELF5

SEQ ID NO: 434 shows a portion of CELF5 transcribed pre mRNA intronic sequence with cryptic exon corresponding to the genomic position chr19, complement: 3278209-3278316.

The lower-case bases denote the bases immediately flanking the splice donor site (gu). The TDP-43 binding region is shown underlined.

GUAGCCCCUGGCUGUCCUUCAGAGGGGGCACAGGUGGAGAAAGAGGCGC
AGUCCCUGGCUGUGGUCCCUGGAGUGGGUAUACACGUGUGAGUGUGUGC
AGAUGUGGAGguGAGUAGGCAAGCGAAGUGUAUGUGUGUGCAUGGAUGU
AUUACAAGUGUGUGCGUGUGGGUGAGUGUGCAUGUCUGGGUGUGAGUGU
GCCCGAGACUGCAUGCAUGUGUGUGUGUGAGU

### CAMK2B

SEQ ID NO: 435 shows a portion of CAMK2B transcribed pre mRNA intronic sequence with cryptic exon corresponding to the genomic position chr7, complement: 44258490-44258514

The lower-case bases denote the bases immediately flanking the splice donor site (gu). The TDP-43 binding region is shown underlined.

CUGGACUGGGGCACCACUGGCUGGCguGAGUGCACAUGUGUGUGUUUAU
GAGUGUGGCUGUGAAAUGUGAGCACGUGCACCUGUAUGUAUGUGUGUGG
GUGUUUGCACGUGGGGGCGCGUGAGCACAUGAAAUCAGGGUCCAUAUGG
GUGGGGAUGUGCAUACAUGUGCAUGUGUAUUGUGUGAAUGUAUGAGUGA
GCGUGUGGAGGUGUGUGCAUGUGG

### UNC13B

SEQ ID NO: 436 shows a portion of UNC13B transcribed pre mRNA intronic sequence with cryptic exon corresponding to the genomic position chr9, complement: 35,364,545-35,364,567. The lower-case bases denote the bases immediately flanking the splice donor site (gu). The TDP-43 binding region is shown underlined.

AAGAAAAGCGAGGAGCCCUUCAGguUGUGCCUAUGACCCUUUGGGUCGU
CCUUUUUUGUCUAUUUCCCUCCCCUCCUUCCCAAGCACUGUAUGUGUGU
GUGUAUGUGUGUGUGUGUGUGUACAUGCACAUGUGCGUGCAUGAUCUGU
GCCUCUGAGCUUUGGCUCAUGCAGUCUAUUUUUCUGAAAAGCAGUUUGU
GUGCAUGC

### Example Target Sequences for Splicing Elements in TDP-43 Regulated Cryptic Exons

The following sequences comprising target sequences. The antisense sequences used in the constructs of the present invention may comprise sequences which are at least 90%, or at least 95%, or at least 100% complementary to these target sequences.

### Example Antisense Sequences for Splicing Elements in TDP-43 Regulated Cryptic Exons

| SEQ ID | Target Sequence | Target site |
|---|---|---|
| SEQ ID NO: 11 | UAAAAUUAUAUUCAUAUUGCagGACUCGGCAGAA | STMN2 2a 3′ splice site |
| | GACCUU | and flanking regions |
| SEQ ID NO: 12 | GUAGAAAAUAAGAAUUUGGCUCUCUGUGUGAGC | STMN2 2a TDP-43 |
| | AUGUGUGCGUGUGUGCGAGAGAGAGAGACAGAC | binding motif and |
| | A | flanking regions |
| SEQ ID NO: 13 | UGUGUGAGCAUGUGUGCGUGUGU | STMN2 2a TDP-43 |
| | | binding motif |
| SEQ ID NO: 14 | AUUCAUAUUGCAGGACUCGGCAGAAGACCUUCG | STMN2 2a ESE and |
| | AGAGAAAGGUAGAA | flanking region (SRSF1) |
| SEQ ID NO: 15 | UAAGAAGAAAUGAAUGUGAAUGCGGCUUGUGGC | STMN2 2a ESE and |
| | ACAGUUGACAAGGAUGAU | flanking region (SRSF2 |
| | | and SRSF6) |
| SEQ ID NO: 16 | AAUGCGGCUUGUGGCACAGUUGACAAGGAUGAU | STMN2 2a ESE and |
| | AAAUCAAUAAUGCA | flanking region (SRSF5) |
| SEQ ID NO: 17 | UGCGGCUUGUGGCACAGUUGACAA | STMN2 ESE target |
| | | region (i.e., comprising |
| | | one or more ESE sites |
| | | identified by ESE finder |
| | | 3.0) |
| SEQ ID NO: 18 | UAAGAAGAAAUGAAUGUGAAUGCGGCUUGUGGC | STMN2 ESE target |
| | ACAGUUGACAAGGAUGAUAAAUCAAUAAUGCA | region and flanking |
| | | region thereof |
| SEQ ID NO: 19 | GGUUCUUCCUUCUAUUCCAGCCCUAACCACUCA | UNC13A 1st 3′ splice |
| | GGAUUGG | site and flanking regions |
| SEQ ID NO: 20 | UGUCUGGGUAUGUCUCUUCCAGCUGCCUGGGUU | UNC13A 3′ splice site |
| | UCCUGGA | and flanking regions |
| SEQ ID NO: 21 | AAGGAUGGAUGGAGAGAUGGGUGAGUACAUGGA | UNC13A 5′ splice site |
| | UGGAUAG | and flanking regions |
| SEQ ID NO: 22 | AAGCAUGGAUGGAGAGAUGGGUGAGUACAUGGA | UNC13A 5′ splice site |
| | UGGAUAG | and flanking regions |
| | | with CE SNP |
| SEQ ID NO: 23 | UGAAUGAAUGAAUGAUUGAACAGAUGAAUGAGUG | UNC13A TDP-43 |
| | AUGAGUAGAUAAAAGGAUGGAUGGAGAGAUGGG | binding region as |
| | UGAGUACAUGGAUGGAUAGAUGGAUGAGUUGGU | determined by iClip |
| | GGGUAGAUUCGUGGCUAGAUGGAUGAUGGAUGG | |
| | AUGGACAGAUGGAUGGAUAUAUGAUUGAACUAU | |
| | UGAAAGUAUAGAUGUAUGGAUGGGUGAAUUUGG | |
| | GGGUAAUUGUUAGAUGAUGGAUGAGUAUAGAUG | |
| | AAUGAUGGAUGGAUAACUUGAUGAGUGGAUAGA | |
| | UAGAUUGCUGGAUAGAUGAUUGACUGGGUGGAU | |
| | AGAUGAAAUGUUGGAUGAGCAGAUUAAGUUGUA | |
| | UUGGAUGGGAUGGAUGGAAGUGUGGUUGAGUUA | |
| | UUAGAAGGAAGAUUGAGUAGAUAGGUGAAUUUG | |
| | UUGAUAGUCAGAUGGGUAGAUAGGUAGAUGGAU | |
| | GGAUGGAUGGAUGGAUGUAUAGGCAGAUGGACA | |
| | AAUGGAUGAAUGGGUGGGUGGAUGAAUGGAAGG | |
| | AUGUGUGGUUGAACUAUUGCAAGUAUUGAUAAU | |
| | UGGGUUCAUAAUUUCUGAAUAUUUAGAUGGAUG | |
| | GUUGUGAGUGGCUGGUGGACAGACGAAAAAUGG | |
| | AUGGUUGGAUAAAUUGAUGGGUGGAUGGAUGGU | |
| | UGGUUGUAUGAAAGAAUGAAUGAUUGGGUAGGU | |
| | GGAUUAAGUUGCGGAUCAAUGUAUGGGAUGGAU | |
| | GAAUGGAUGGAUGGAUGGAUGUGUGGUUGAAUU | |
| | ACUGAAAGGUUGGAAGAGUGGAUGGGUGAAAUU | |
| | UGGGGUAGUUAGAUGGGUGGGUGUGUGGAUGG | |
| | AUAAAAGAGUAGAUGAAUG | |
| SEQ ID NO: 24 | GUUUGUUGAAUAAAUGCUGGUGAAUGAAUGAAU | Shorter UNC13A TDP- |
| | GAUUGAACAGAUGAAUGAGUGAUGAGUAGAUAAA | 43 binding region and |
| | AGGAUGGAUGGAGAGAUGGguGAGUACAUGGAU | flanking regions without |
| | GGAUAGAU | CE SNP |
| SEQ ID NO: 25 | GUUUGUUGAAUAAAUGCUGGUGAAUGAAUGAAU | Shorter UNC13A TDP- |
| | GAUUGAACAGAUGAAUGAGUGAUGAGUAGAUAAA | 43 binding region and |
| | AGCAUGGAUGGAGAGAUGGGUGAGUACAUGGAU | flanking regions with CE |
| | GGAUAGAU | SNP |
| SEQ ID NO: 26 | GGAUGGAUGGAGAGAUGGGUGA | UNC13A shorter TDP- |
| | | 43 binding region |
| | | comprising UG-rich |
| | | motifs |
| SEQ ID NO: 27 | CUUCUAUUCCAGCCCUAACCACUCAGGAUUGGG | UNC13A ESE and |
| | CCGUUUGUGUCUGGG | flanking region (SRSF1 |
| | | and SRSF5) |
| SEQ ID NO: 28 | UGUCUCUUCCAGCUGCCUGGGUUUCCUGGAAAG | UNC13A ESE and |
| | AACUCUUAUCCCCAG | flanking region (SRSF2) |
| SEQ ID NO: 29 | CCACUCAGGAUUGGGCCGUUUGUGUCUGGGUAU | UNC13A ESE and |
| | GUCUCUUCCAGCU | flanking region (SRSF6) |
| SEQ ID NO: 30 | ACUCAGGAUUGGGCCGUUUGUGUCUGGGUAUGU | UNC13A target ESE |
| | CUCUUCCAGCUGCCUGGGUUUCCUG | region (i.e., comprising |
| | | one or more ESE sites |
| | | identified by ESE finder |
| | | 3.0) |
| SEQ ID NO: 31 | CUAUGCUUACUGUGCCAUAUAGUACCGGAUACG | INSR 3′ splice site and |
| | GGAUGAA | flanking regions |
| SEQ ID NO: 32 | GGAUACGGGAUGAAGUCAUACAAGCACUGAAUG | INSR TDP-43 binding |
| | AAUGGAUGAAUGAAUGAUGGAUGAAUGGAUGAC | motif and flanking |
| | ACCUUCUUAUAUGUAUCAGGCUGAUGCUGAAGA | regions |
| | C | |
| SEQ ID NO: 33 | UGAAUGAAUGGAUGAAUGAAUGAUGGAUGAAUG | INSR TDP-43 binding |
| | GAUGACACCUUCUUAUAUGU | motif |
| SEQ ID NO: 34 | UGAAUGAUGGAUGAAUGGAUGACACCUUCUUAU | INSR ESE and flanking |
| | AUGUGUAUCAGGCU | region (SRSF1) |
| SEQ ID NO: 35 | AUAUGUGUAUCAGGCUGAUGCUGAAGACUUCAA | INSR ESE and flanking |
| | AGUUGAGUAAAAUA | region (SRSF1) |
| SEQ ID NO: 36 | CACUGAAUGAAUGGAUGAAUGAAUGAUGGAUGAA | INSR ESE and flanking |
| | UGGAUGACACCUUC | region (SRSF2) |
| SEQ ID NO: 37 | ACCUUCUUAUAUGUGUAUCAGGCUGAUGCUGAA | INSR ESE and flanking |
| | GACUUCAAAGUUGAGUAAAAUA | region (SRSF2) |
| SEQ ID NO: 38 | GUACCGGAUACGGGAUGAAGUCAUACAAGCACU | INSR ESE and flanking |
| | GAAUGAAUGGAUGAAU | region (SRSF5) |
| SEQ ID NO: 39 | CUGUGCCAUAUagUACCGGAUACGGGAUGAAGUC | INSR ESE and flanking |
| | AUACAAGCACUG | region (SRSF6) |
| SEQ ID NO: 40 | AUGGAUGACACCUUCUUAUAUGUGUAUCAGGCU | INSR ESE and flanking |
| | GAUGCUGAAGACU | region (SRSF6) |
| SEQ ID NO: 437 | GACGUGCUUAUUAUUUGACagGUGCAUGUGACA | ELALV3 3′ splice site |
| | CUGUGACU | and flanking region |
| SEQ ID NO: 438 | ACGUGCUUAUUAUUUGACagGUGCAUGUGACACU | ELALV3 TDP-43 |
| | GUGACUCCGGCUGUGACCUGAUGGG | binding region and |
| | | flanking region thereof |
| SEQ ID NO: 439 | GGCCUCAGGGAUGCGUCUGGCUCUGGCAGGAU | ELALV3 TDP-43 |
| | GUUUGUGUGUCACCGCGAUGUUGUGUGGG | binding region and |
| | | flanking region thereof. |
| SEQ ID NO: 440 | UGUGUCACCGCGAUGUUGUGUGGGUGUGUCUA | ELALV3 TDP-43 |
| | CCUGUGCCCUGCUCUGAGGGAUUGAGUGUG | binding region and |
| | | flanking region thereof. |
| SEQ ID NO: 441 | UUUUCCCUUACACCUUGACCagCUUGCAUAUUGG | G3BP1 3′ splice site |
| | AUACCACA | and flanking region |
| | | thereof |
| SEQ ID NO: 442 | ACACCUUGACCagCUUGCAUAUUGGAUACCAC | G3BP1 TDP-43 binding |
| | | region |
| SEQ ID NO: 443 | UGUGUGUGUGUGUGUCACCCagGCUGGAGUGCA | AARS1 3′ splice site |
| | GUGGCAUGA | and flanking region |
| | | thereof. |
| SEQ ID NO: 444 | AUCUUUUGUGUGUGUGUGUGUGUGUGUGUGUG | AARS1 TDP-43 binding |
| | UGUGUGUGUCACCCagGCUGGAGUGCAGUGGCA | region |
| | UGAUCACAGCUCACUGCAGCCUCAACUUCCU | |
| SEQ ID NO: 445 | AGUGAUCCUCUCCCGAGUAGCUGGGACUACAG | AARS1 TDP-43 binding |
| | | region |
| SEQ ID NO: 446 | GUGUGUGCAGAUGUGGAGguGAGUAGGCAAGCG | CELF5 5′ splice site |
| | AAGUGUA | and flanking region |
| | | thereof. |
| SEQ ID NO: 447 | AGGCAAGCGAAGUGUAUGUGUGUGCAUGGAUGU | CELF5 TDP-43 binding |
| | AUUACAAGUGUGUGCGUGUGGGUGAGUG | region and flanking |
| | | regions thereof |
| SEQ ID NO: 448 | CAAGUGUGUGCGUGUGGGUGAGUGUGCAUGUC | CELF5 TDP-43 binding |
| | UGGGUGUGAGUGUGCCCGAGA | region and flanking |
| | | regions thereof |
| SEQ ID NO: 449 | AGUGUGCCCGAGACUGCAUGCAUGUGUGUGUGU | CELF5 TDP-43 binding |
| | GAGU | region and flanking |
| | | regions thereof |
| SEQ ID NO: 450 | GGGGCACCACUGGCUGGCguGAGUGCACAUGUG | CAMK2B 5′ splice site |
| | UGUGUUU | and flanking regions |
| | | thereof |
| SEQ ID NO: 451 | ACAUGUGCAUGUGUAUUGUGUGAAUGUAUGAGU | CAMK2B TDP-43 |
| | GAGCGUGUGGAGGUGUGUGCAUGUGG | binding region and |
| | | flanking regions thereof |
| SEQ ID NO: 452 | AAGAAAAGCGAGGAGCCCUUCAGguUGUGCCUAU | UNC13B 5′ splice site |
| | GACCCUUUGGG | and flanking regions |
| | | thereof |
| SEQ ID NO: 453 | UCCCAAGCACUGUAUGUGUGUGUGUAUGUGUGU | UNC13B TDP-43 |
| | GUGUGUGUGUACAUGCACAUGUGCGUGCAUGAU | binding region and |
| | CUGUGCCUCUGAGCUUUGGCUCAUGC | flanking regions thereof |
| SEQ ID NO: 454 | CAUGCAGUCUAUUUUUCUGAAAAGCAGUUUGUG | UNC13B TDP-43 |
| | UGCAUGC | binding region and |
| | | flanking regions thereof |

Example Antisense Sequences that Target the STMN2 3′ Splice Site/Splice Acceptor Site

| | |
|---|---|
| SEQ ID NO: 42 | GUCUUCUGCCGAGUCC |
| SEQ ID NO: 43 | GUCUUCUGCCGAGUCc |
| SEQ ID NO: 44 | UCUUCUGCCGAGUCcU |
| SEQ ID NO: 45 | CUUCUGCCGAGUCcUG |
| SEQ ID NO: 46 | UUCUGCCGAGUCcUGC |
| SEQ ID NO: 47 | UCUGCCGAGUCcUGCA |
| SEQ ID NO: 48 | CUGCCGAGUCcUGCAA |
| SEQ ID NO: 49 | UGCCGAGUCcUGCAAU |
| SEQ ID NO: 50 | GCCGAGUCcUGCAAUA |
| SEQ ID NO: 51 | CCGAGUCcUGCAAUAU |
| SEQ ID NO: 52 | CGAGUCcUGCAAUAUG |
| SEQ ID NO: 53 | GAGUCcUGCAAUAUGA |
| SEQ ID NO: 54 | AGUCcUGCAAUAUGAA |
| SEQ ID NO: 55 | GCCcUGCAAUAUGAAU |
| SEQ ID NO: 56 | CCCUGCAAUAUGAAUA |
| SEQ ID NO: 57 | CcUGCAAUAUGAAUAU |
| SEQ ID NO: 58 | cUGCAAUAUGAAUAUA |
| SEQ ID NO: 59 | UGCAAUAUGAAUAUAA |

Example Antisense Sequences that Target the STMN2 TDP-43 Binding Motif and Flanking Regions Thereof

| | |
|---|---|
| SEQ ID NO: 60 | UGUCUGUCUCUCUCUC |
| SEQ ID NO: 61 | GUCUGUCUCUCUCUCU |
| SEQ ID NO: 62 | UCUGUCUCUCUCUCUC |
| SEQ ID NO: 63 | CUGUCUCUCUCUCUCG |
| SEQ ID NO: 64 | UGUCUCUCUCUCUCGC |
| SEQ ID NO: 65 | GUCUCUCUCUCUCGCa |
| SEQ ID NO: 66 | UCUCUCUCUCUCGCac |
| SEQ ID NO: 67 | CUCUCUCUCUCGCaca |
| SEQ ID NO: 68 | UCUCUCUCUCGCacac |
| SEQ ID NO: 69 | CUCUCUCUCGCacaca |
| SEQ ID NO: 70 | UCUCUCUCGCacacac |
| SEQ ID NO: 71 | CUCUCUCGCacacacg |
| SEQ ID NO: 72 | UCUCUCGCacacacgc |
| SEQ ID NO: 73 | CUCUCGCacacacgca |
| SEQ ID NO: 74 | UCUCGCacacacgcac |
| SEQ ID NO: 75 | CUCGCacacacgcaca |
| SEQ ID NO: 76 | UCGCacacacgcacac |
| SEQ ID NO: 77 | CGCacacacgcacaca |
| SEQ ID NO: 78 | GCacacacgcacacaU |
| SEQ ID NO: 79 | CacacacgcacacaUg |
| SEQ ID NO: 80 | acacacgcacacaUgc |
| SEQ ID NO: 81 | cacacgcacacaUgcU |
| SEQ ID NO: 82 | acacgcacacaUgcUc |
| SEQ ID NO: 83 | cacgcacacaUgcUca |
| SEQ ID NO: 84 | acgcacacaUgcUcac |
| SEQ ID NO: 85 | cgcacacaUgcUcaca |
| SEQ ID NO: 86 | gcacacaUgcUcacac |
| SEQ ID NO: 87 | cacacaUgcUcacaca |
| SEQ ID NO: 88 | acacaUgcUcacacaG |
| SEQ ID NO: 89 | cacaUgcUcacacaGA |
| SEQ ID NO: 90 | acaUgcUcacacaGAG |
| SEQ ID NO: 91 | caUgcUcacacaGAGA |
| SEQ ID NO: 92 | aUgcUcacacaGAGAG |
| SEQ ID NO: 93 | UgcUcacacaGAGAGC |
| SEQ ID NO: 94 | gcUcacacaGAGAGCC |
| SEQ ID NO: 95 | cUcacacaGAGAGCCA |
| SEQ ID NO: 96 | UcacacaGAGAGCCAA |
| SEQ ID NO: 97 | cacacaGAGAGCCAAA |
| SEQ ID NO: 98 | acacaGAGAGCCAAAU |
| SEQ ID NO: 99 | cacaGAGAGCCAAAUU |
| SEQ ID NO: 100 | acaGAGAGCCAAAUUC |
| SEQ ID NO: 101 | caGAGAGCCAAAUUCU |
| SEQ ID NO: 102 | aGAGAGCCAAAUUCUU |

Example Antisense Sequences that Target the First UNC13A 3′ Splice Site/Splice Acceptor Site

| | |
|---|---|
| SEQ ID NO: 103 | CCUGAGUGGUUAGGGc |
| SEQ ID NO: 104 | CUGAGUGGUUAGGGcU |
| SEQ ID NO: 105 | UAGUGGUUAGGGcUG |
| SEQ ID NO: 106 | AGUGGUUAGGGcUGG |
| SEQ ID NO: 107 | GUGGUUAGGGcUGGA |
| SEQ ID NO: 108 | UGGUUAGGGcUGGAA |
| SEQ ID NO: 109 | GGUUAGGGcUGGAAU |
| SEQ ID NO: 110 | GUUAGGGcUGGAAUA |
| SEQ ID NO: 111 | UUAGGGcUGGAAUAG |
| SEQ ID NO: 112 | UAGGGcUGGAAUAGA |
| SEQ ID NO: 113 | AGGGcUGGAAUAGAA |
| SEQ ID NO: 114 | GGGcUGGAAUAGAAG |
| SEQ ID NO: 115 | GGcUGGAAUAGAAGG |
| SEQ ID NO: 116 | GcUGGAAUAGAAGGAA |
| SEQ ID NO: 117 | cUGGAAUAGAAGGAAU |
| SEQ ID NO: 118 | cUGGAAUAGAAGGAAUA |

Example Antisense Sequences that Target the Second UNC13A 3′ Splice Site/Splice Acceptor Site

| | |
|---|---|
| SEQ ID NO: 119 | AGGAAACCCAGGCAGc |
| SEQ ID NO: 120 | GGAAACCCAGGCAGCU |
| SEQ ID NO: 121 | GAAACCCAGGCAGcUG |
| SEQ ID NO: 122 | AAACCCAGGCAGcUGG |
| SEQ ID NO: 123 | AACCCAGGCAGcUGGA |
| SEQ ID NO: 124 | ACCCAGGCAGcUGGAA |
| SEQ ID NO: 125 | CCCAGGCAGcUGGAAG |
| SEQ ID NO: 126 | CCAGGCAGcUGGAAGA |
| SEQ ID NO: 127 | CAGGCAGcUGGAAGAG |
| SEQ ID NO: 128 | AGGCAGcUGGAAGAGA |
| SEQ ID NO: 129 | GGCAGcUGGAAGAGAC |
| SEQ ID NO: 130 | GCAGcUGGAAGAGACA |
| SEQ ID NO: 131 | CAGcUGGAAGAGACAU |
| SEQ ID NO: 132 | AGcUGGAAGAGACAUA |
| SEQ ID NO: 133 | GcUGGAAGAGACAUAC |
| SEQ ID NO: 134 | cUGGAAGAGACAUACC |
| SEQ ID NO: 135 | UGGAAGAGACAUACCC |

Example Antisense Sequences that Target the UNC13A 5′ Splice Site/Splice Donor Site

| | |
|---|---|
| SEQ ID NO: 136 | UCCAUCCAUGUACUCa |
| SEQ ID NO: 137 | CCAUCCAUGUACUCac |
| SEQ ID NO: 138 | CAUCCAUGUACUCacC |
| SEQ ID NO: 139 | AUCCAUGUACUCacCC |
| SEQ ID NO: 140 | UCCAUGUACUCacCCA |
| SEQ ID NO: 141 | CCAUGUACUCacCCAU |
| SEQ ID NO: 142 | CAUGUACUCacCCAUC |
| SEQ ID NO: 143 | AUGUACUCacCCAUCU |
| SEQ ID NO: 144 | UGUACUCacCCAUCUC |
| SEQ ID NO: 145 | GUACUCacCCAUCUCU |
| SEQ ID NO: 146 | UACUCacCCAUCUCUC |
| SEQ ID NO: 147 | ACUCacCCAUCUCUCC |
| SEQ ID NO: 148 | CUCacCCAUCUCUCCA |
| SEQ ID NO: 149 | UCacCCAUCUCUCCAU |
| SEQ ID NO: 150 | CacCCAUCUCUCCAUC |
| SEQ ID NO: 151 | acCCAUCUCUCCAUCC |
| SEQ ID NO: 152 | cCCAUCUCUCCAUCCA |

Example Antisense Sequences that Target the UNC13A TDP-43 Binding Region.

| | |
|---|---|
| SEQ ID NO: 153 | AUCUAUCCAUCCAUGU |
| SEQ ID NO: 154 | UCUAUCCAUCCAUGUA |
| SEQ ID NO: 155 | CUAUCCAUCCAUGUAC |
| SEQ ID NO: 156 | UAUCCAUCCAUGUACU |
| SEQ ID NO: 157 | AUCCAUCCAUGUACUC |
| SEQ ID NO: 158 | UCCAUCCAUGUACUCA |
| SEQ ID NO: 159 | CCAUCCAUGUACUCAC |
| SEQ ID NO: 160 | CAUCCAUGUACUCACC |
| SEQ ID NO: 161 | AUCCAUGUACUCAcCC |
| SEQ ID NO: 162 | UCCAUGUACUCAcCCA |
| SEQ ID NO: 163 | CCAUGUACUCAcCCAU |
| SEQ ID NO: 164 | AUGUACUCAcCCAUC |
| SEQ ID NO: 165 | UGUACUCAcCCAUCU |
| SEQ ID NO: 166 | GUACUCAcCCAUCUC |
| SEQ ID NO: 167 | UACUCAcCCAUCUCU |
| SEQ ID NO: 168 | ACUCAcCCAUCUCUC |
| SEQ ID NO: 169 | CUCAcCCAUCUCUCC |
| SEQ ID NO: 170 | UCAcCCAUCUCUCCA |
| SEQ ID NO: 171 | CAcCCAUCUCUCCAU |
| SEQ ID NO: 172 | AcCCAUCUCUCCAUC |
| SEQ ID NO: 173 | cCCAUCUCUCCAUCC |
| SEQ ID NO: 174 | CCAUCUCUCCAUCCA |
| SEQ ID NO: 175 | CAUCUCUCCAUCCAU |
| SEQ ID NO: 176 | AUCUCUCCAUCCAUC |
| SEQ ID NO: 177 | AUCUCUCCAUCCAUG |
| SEQ ID NO: 178 | UCUCUCCAUCCAUCC |
| SEQ ID NO: 179 | UCUCUCCAUCCAUGC |
| SEQ ID NO: 180 | CUCUCCAUCCAUCCU |
| SEQ ID NO: 181 | CUCUCCAUCCAUGCU |
| SEQ ID NO: 182 | UCUCCAUCCAUCCUU |
| SEQ ID NO: 183 | UCUCCAUCCAUGCUU |
| SEQ ID NO: 184 | CUCCAUCCAUCCUUU |
| SEQ ID NO: 185 | CUCCAUCCAUGCUUU |
| SEQ ID NO: 186 | UCCAUCCAUCCUUUU |
| SEQ ID NO: 187 | UCCAUCCAUGCUUUU |
| SEQ ID NO: 188 | CCAUCCAUCCUUUUA |
| SEQ ID NO: 189 | CCAUCCAUGCUUUUA |
| SEQ ID NO: 190 | CAUCCAUCCUUUUAU |
| SEQ ID NO: 191 | CAUCCAUGCUUUUAU |
| SEQ ID NO: 192 | AUCCAUCCUUUUAUC |
| SEQ ID NO: 193 | AUCCAUGCUUUUAUC |
| SEQ ID NO: 194 | UCCAUCCUUUUAUCU |
| SEQ ID NO: 195 | UCCAUGCUUUUAUCU |
| SEQ ID NO: 196 | CCAUCCUUUUAUCUA |
| SEQ ID NO: 197 | CCAUGCUUUUAUCUA |
| SEQ ID NO: 198 | CAUCCUUUUAUCUAC |
| SEQ ID NO: 199 | CAUGCUUUUAUCUAC |
| SEQ ID NO: 200 | AUCCUUUUAUCUACU |
| SEQ ID NO: 201 | AUGCUUUUAUCUACU |
| SEQ ID NO: 202 | UCCUUUUAUCUACUC |
| SEQ ID NO: 203 | UGCUUUUAUCUACUC |
| SEQ ID NO: 204 | CCUUUUAUCUACUCA |
| SEQ ID NO: 205 | GCUUUUAUCUACUCA |
| SEQ ID NO: 206 | CUUUUAUCUACUCAU |
| SEQ ID NO: 207 | UUUUAUCUACUCAUC |
| SEQ ID NO: 208 | UUUAUCUACUCAUCA |
| SEQ ID NO: 209 | UUAUCUACUCAUCAC |
| SEQ ID NO: 210 | UAUCUACUCAUCACU |
| SEQ ID NO: 211 | AUCUACUCAUCACUC |
| SEQ ID NO: 212 | UCUACUCAUCACUCA |
| SEQ ID NO: 213 | CUACUCAUCACUCAU |
| SEQ ID NO: 214 | UACUCAUCACUCAUU |
| SEQ ID NO: 215 | ACUCAUCACUCAUUC |
| SEQ ID NO: 216 | CUCAUCACUCAUUCA |
| SEQ ID NO: 217 | UCAUCACUCAUUCAU |
| SEQ ID NO: 218 | CAUCACUCAUUCAUC |
| SEQ ID NO: 219 | AUCACUCAUUCAUCU |
| SEQ ID NO: 220 | UCACUCAUUCAUCUG |
| SEQ ID NO: 221 | CACUCAUUCAUCUGU |
| SEQ ID NO: 222 | ACUCAUUCAUCUGUU |
| SEQ ID NO: 223 | CUCAUUCAUCUGUUC |
| SEQ ID NO: 224 | UCAUUCAUCUGUUCA |
| SEQ ID NO: 225 | CAUUCAUCUGUUCAA |
| SEQ ID NO: 226 | AUUCAUCUGUUCAAU |
| SEQ ID NO: 227 | UUCAUCUGUUCAAUC |
| SEQ ID NO: 228 | UCAUCUGUUCAAUCA |
| SEQ ID NO: 229 | CAUCUGUUCAAUCAU |
| SEQ ID NO: 230 | AUCUGUUCAAUCAUU |
| SEQ ID NO: 231 | UCUGUUCAAUCAUUC |
| SEQ ID NO: 232 | CUGUUCAAUCAUUCA |
| SEQ ID NO: 233 | UGUUCAAUCAUUCAU |
| SEQ ID NO: 234 | GUUCAAUCAUUCAUU |
| SEQ ID NO: 235 | UUCAAUCAUUCAUUC |
| SEQ ID NO: 236 | UCAAUCAUUCAUUCA |
| SEQ ID NO: 237 | CAAUCAUUCAUUCAU |
| SEQ ID NO: 238 | AAUCAUUCAUUCAUU |
| SEQ ID NO: 239 | AUCAUUCAUUCAUUC |
| SEQ ID NO: 240 | UCAUUCAUUCAUUCA |
| SEQ ID NO: 241 | CAUUCAUUCAUUCAC |
| SEQ ID NO: 242 | AUUCAUUCAUUCACC |
| SEQ ID NO: 243 | UUCAUUCAUUCACCA |
| SEQ ID NO: 244 | UCAUUCAUUCACCAG |
| SEQ ID NO: 245 | CAUUCAUUCACCAGC |
| SEQ ID NO: 246 | AUUCAUUCACCAGCA |
| SEQ ID NO: 247 | UUCAUUCACCAGCAU |
| SEQ ID NO: 248 | UCAUUCACCAGCAUU |
| SEQ ID NO: 249 | CAUUCACCAGCAUUU |
| SEQ ID NO: 250 | AUUCACCAGCAUUUA |
| SEQ ID NO: 251 | UUCACCAGCAUUUAU |
| SEQ ID NO: 252 | UCACCAGCAUUUAUU |
| SEQ ID NO: 253 | CACCAGCAUUUAUUC |
| SEQ ID NO: 254 | ACCAGCAUUUAUUCA |
| SEQ ID NO: 255 | CCAGCAUUUAUUCAA |
| SEQ ID NO: 256 | CAGCAUUUAUUCAAC |
| SEQ ID NO: 257 | AGCAUUUAUUCAACA |
| SEQ ID NO: 258 | GCAUUUAUUCAACAA |
| SEQ ID NO: 259 | CAUUUAUUCAACAAA |
| SEQ ID NO: 260 | AUUUAUUCAACAAAC |

Example Antisense Sequences that Target the INSR 3′ Splice Site/Splice Acceptor Site

| | |
|---|---|
| SEQ ID NO: 261 | AUCCCGUAUCCGGUAc |
| SEQ ID NO: 262 | UCCCGUAUCCGGUACU |
| SEQ ID NO: 263 | CCCGUAUCCGGUACUA |
| SEQ ID NO: 264 | CCGUAUCCGGUACUAU |
| SEQ ID NO: 265 | CGUAUCCGGUAcUAUA |
| SEQ ID NO: 266 | GUAUCCGGUACUAUAU |
| SEQ ID NO: 267 | UAUCCGGUACUAUAUG |
| SEQ ID NO: 268 | AUCCGGUAcUAUAUGG |
| SEQ ID NO: 269 | UCCGGUACUAUAUGGC |
| SEQ ID NO: 270 | CCGGUAUAUAUGGCA |
| SEQ ID NO: 271 | CGGUAcUAUAUGGCAC |
| SEQ ID NO: 272 | GGUAcUAUAUGGCACA |
| SEQ ID NO: 273 | AUACUAUAUGGCACAG |
| SEQ ID NO: 274 | UAUAUAUGGCACAGU |
| SEQ ID NO: 275 | AcUAUAUGGCACAGUA |
| SEQ ID NO: 276 | CUAUAUGGCACAGUAA |
| SEQ ID NO: 277 | UAUAUGGCACAGUAAG |

Example Antisense Sequences that Target the INSR TDP-43 Binding Region and Flanking Regions Thereof.

| | |
|---|---|
| SEQ ID NO: 278 | GUCUUCAGCAUCAGCC |
| SEQ ID NO: 279 | UCUUCAGCAUCAGCCU |
| SEQ ID NO: 280 | CUUCAGCAUCAGCCUG |
| SEQ ID NO: 281 | UUCAGCAUCAGCCUGA |
| SEQ ID NO: 282 | UCAGCAUCAGCCUGAU |
| SEQ ID NO: 283 | CAGCAUCAGCCUGAUA |
| SEQ ID NO: 284 | AGCAUCAGCCUGAUAC |
| SEQ ID NO: 285 | GCAUCAGCCUGAUACA |
| SEQ ID NO: 286 | CAUCAGCCUGAUACAU |
| SEQ ID NO: 287 | AUCAGCCUGAUACAUA |
| SEQ ID NO: 288 | UCAGCCUGAUACAUAU |
| SEQ ID NO: 289 | CAGCCUGAUACAUAUA |
| SEQ ID NO: 290 | AGCCUGAUACAUAUAA |
| SEQ ID NO: 291 | GCCUGAUACAUAUAAG |
| SEQ ID NO: 292 | CCUGAUACAUAUAAGA |
| SEQ ID NO: 293 | CUGAUACAUAUAAGAA |
| SEQ ID NO: 294 | UGAUACAUAUAAGAAG |
| SEQ ID NO: 295 | GAUACAUAUAAGAAGG |
| SEQ ID NO: 296 | AUACAUAUAAGAAGGU |
| SEQ ID NO: 297 | UACAUAUAAGAAGGUG |
| SEQ ID NO: 298 | ACAUAUAAGAAGGUG |
| SEQ ID NO: 299 | CAUAUAAGAAGGUGU |
| SEQ ID NO: 300 | ACAUAUAAGAAGGUGU |
| SEQ ID NO: 301 | CAUAUAAGAAGGUGUC |
| SEQ ID NO: 302 | AUAUAAGAAGGUGUCA |
| SEQ ID NO: 303 | UAUAAGAAGGUGUCAU |
| SEQ ID NO: 304 | AUAAGAAGGUGUCAUC |
| SEQ ID NO: 305 | UAAGAAGGUGUCAUCC |
| SEQ ID NO: 306 | AAGAAGGUGUCAUCCA |
| SEQ ID NO: 307 | AGAAGGUGUCAUCCAU |
| SEQ ID NO: 308 | GAAGGUGUCAUCCAUU |
| SEQ ID NO: 309 | AAGGUGUCAUCCAUUC |
| SEQ ID NO: 310 | AGGUGUCAUCCAUUCA |
| SEQ ID NO: 311 | GGUGUCAUCCAUUCAU |
| SEQ ID NO: 312 | GUGUCAUCCAUUCAUC |
| SEQ ID NO: 313 | UGUCAUCCAUUCAUCC |
| SEQ ID NO: 314 | GUCAUCCAUUCAUCCA |
| SEQ ID NO: 315 | UCAUCCAUUCAUCCAU |
| SEQ ID NO: 316 | CAUCCAUUCAUCCAUC |
| SEQ ID NO: 317 | AUCCAUUCAUCCAUCA |
| SEQ ID NO: 318 | UCCAUUCAUCCAUCAU |
| SEQ ID NO: 319 | CCAUUCAUCCAUCAUU |
| SEQ ID NO: 320 | CAUUCAUCCAUCAUUC |
| SEQ ID NO: 321 | AUUCAUCCAUCAUUCA |
| SEQ ID NO: 322 | UUCAUCCAUCAUUCAU |
| SEQ ID NO: 323 | UCAUCCAUCAUUCAUU |
| SEQ ID NO: 324 | CAUCCAUCAUUCAUUC |
| SEQ ID NO: 325 | AUCCAUCAUUCAUUCA |
| SEQ ID NO: 326 | UCCAUCAUUCAUUCAU |
| SEQ ID NO: 327 | CCAUCAUUCAUUCAUC |
| SEQ ID NO: 328 | CAUCAUUCAUUCAUCC |
| SEQ ID NO: 329 | AUCAUUCAUUCAUCCA |
| SEQ ID NO: 330 | UCAUUCAUUCAUCCAU |
| SEQ ID NO: 331 | CAUUCAUUCAUCCAUU |
| SEQ ID NO: 332 | AUUCAUUCAUCCAUUC |
| SEQ ID NO: 333 | UUCAUUCAUCCAUUCA |
| SEQ ID NO: 334 | UCAUUCAUCCAUUCAU |
| SEQ ID NO: 235 | CAUUCAUCCAUUCAUU |
| SEQ ID NO: 236 | AUUCAUCCAUUCAUUC |
| SEQ ID NO: 237 | UUCAUCCAUUCAUUCA |
| SEQ ID NO: 238 | UCAUCCAUUCAUUCAG |
| SEQ ID NO: 239 | CAUCCAUUCAUUCAGU |
| SEQ ID NO: 340 | AUCCAUUCAUUCAGUG |
| SEQ ID NO: 341 | UCCAUUCAUUCAGUGC |
| SEQ ID NO: 342 | CCAUUCAUUCAGUGCU |
| SEQ ID NO: 343 | CAUUCAUUCAGUGCUU |
| SEQ ID NO: 344 | AUUCAUUCAGUGCUUG |
| SEQ ID NO: 345 | UUCAUUCAGUGCUUGU |
| SEQ ID NO: 346 | UCAUUCAGUGCUUGUA |
| SEQ ID NO: 347 | CAUUCAGUGCUUGUAU |
| SEQ ID NO: 348 | AUUCAGUGCUUGUAUG |
| SEQ ID NO: 349 | UUCAGUGCUUGUAUGA |
| SEQ ID NO: 350 | UCAGUGCUUGUAUGAC |
| SEQ ID NO: 351 | CAGUGCUUGUAUGACU |
| SEQ ID NO: 352 | AGUGCUUGUAUGACUU |

### Example 1: Constructs Targeting the UNC13A Cryptic Exon

Example constructs targeting UNC13A cryptic splicing were designed to all comprise an antisense sequence that binds to a splicing element of the TDP-43 regulated UNC13A cryptic exon.

### Example 1A

SEQ ID NO 358:
AACUAUCCAUCCAUGUACUCACCCAAUUUUUGGAGCAGGUUUUCUGAC
UUCGGUCGGAAAACCCCU

This sequence comprises the following antisense sequence SEQ ID NO 359: CUAUCCAUCCAUGUACUCACCC (shown above in bold). This sequence targets the 5′ splice site (donor site) of the TDP-43 regulated UNC13A cryptic exon.

An U7 SmOPT core expression cassette was generated by gene synthesis cloned either in pUC-Simple (General Biosystems) or in a pMK vector followed by a f1 origin and a CMV promoter driving a Blasticidin resistance cDNA followed by an SV40 polyadylation signal (GeneArt, Life technologies):

The complete U7 SmOPT Cassette for Example 1A is as follows: (SEQ ID NO: 360)

GAAUUCCAACAUAGGAGCUGUGAUUGGCUGUUUUCAGCCAAUCAGCAC
UGACUCAUUUGCAUAGCCUUUACAAGCGGUCACAAACUCAAGAAACGA
GCGGUUUUAAUAGUCUUUUAGAAUAUUGUUUAUCGAACCGAAUAAGGA
ACUGUGCUUUGUGAUUCACAUAUCAGUGGAGGGGUGUGGAAAUGGCAC
CUUGAUCUCACCCUCAUCGAAAGUGGAGUUGAUGUCCUUCCCUGGCUC
GCUACAGAGGCCUUUCCGCAACUAUCCAUCCAUGUACUCACCCAAUUU
UUGGAGCAGGUUUUCUGACUUCGGUCGGAAAACCCCUCCCAAGUUAAC
UGGUCUACAAUGAAAGCAAAACAGUUCUCUUCCCCGCUCCCCGGUGUG
UGAGAGGGGCUUUGAUCCUUCUCUGGUUUCCUAGGAAACGCGUAAGCU
U

The construct includes the following components:

Mouse U7 promoter (this initiates transcription; note only UsnRNA promoters can drive expression of U snRNAs but other promoters different to the one described below can be used—SEQ ID NO: 41

AACAUAGGAGCUGUGAUUGGCUGUUUUCAGCCAAUCAGCACUGACUCA
UUUGCAUAGCCUUUACAAGCGGUCACAAACUCAAGAAACGAGCGGUUU
UAAUAGUCUUUUAGAAUAUUGUUUAUCGAACCGAAUAAGGAACUGUGC
UUUGUGAUUCACAUAUCAGUGGAGGGGUGUGGAAAUGGCACCUUGAUC
UCACCCUCAUCGAAAGUGGAGUUGAUGUCCUUCCCUGGCUCGCUACAG
AGGCCUUUCCGC

### Transcription Start Site

### A (Shown Underlined Above)

Antisense Sequence
SEQ ID NO: 359
CUAUCCAUCCAUGUACUCACCC
SmOPT site
SEQ ID NO: 355
AAUUUUUGGAG
3′ Hairpin
SEQ ID NO: 356
CAGGUUUUCUGACUUCGGUCGGAAAACCCCU
3′box (for 3′end formation of the snRNA)
SEQ ID NO: 357
GUCUACAAUGAAAG

Everything in this expression cassette except the antisense sequence is the same for all other example constructs.

Example 1B
SEQ ID NO 361
AAUACUCACCCAUCUCUCCAUCCAAUUUUUGGAGCAGGUUUUCUGACUUCGGUCGG
AAAACCCCU
This sequence comprises a different antisense sequence
(shown above in bold) which targets the 5′ splice site
(donor site) of the TDP-43 regulated UNC13A cryptic exon.
SEQ ID NO 362:
UACUCACCCAUCUCUCCAUCC
Example 1C
SEQ ID NO 363:
AAUGUACUCACCCAUCUCUCCAAUUUUUGGAGCAGGUUUUCUGACUUCGGUCGGAA
AACCCCU
This sequence comprises the antisense sequence (shown above in
bold)which targets the 5′ splice site (donor site) of the
TDP-43 regulated UNC13A cryptic exon.
SEQ ID NO 364:
AUGUACUCACCCAUCUCUCC
Example 1D
SEQ ID NO 365:
AACAUCCAUGUACUCACCCAUCUCAAUUUUUGGAGCAGGUUUUCUGACUUCGGUCG
GAAAACCCCU
This sequence comprises the antisense sequence (shown above in
bold)which targets the 5′ splice site (donor site) of the TDP-43
regulated UNC13A cryptic exon.
SEQ ID NO 366:
CAUCCAUGUACUCACCCAUCUC
Example 1E
SEQ ID NO 367:
AAUCCAUCCAUGUACUCACCCAUCUCAAUUUUUGGAGCAGGUUUUCUGACUUCGGU
CGGAAAACCCCU
This sequence comprises the antisense sequence (shown above in
bold) which targets the 5′ splice site (donor site) of the
TDP-43 regulated UNC13A cryptic exon.
SEQ ID NO 368:
UCCAUCCAUGUACUCACCCAUCUC
Example 1F
SEQ ID NO 369:
AAUCCAUGUACUCACCCAUCUCAAUUUUUGGAGCAGGUUUUCUGACUUCGGUCGGA
AAACCCCU
This sequence comprises the antisense sequence (shown above
in bold) which targets the 5′ splice site (donor site) of the
TDP-43 regulated UNC13A cryptic exon.
SEQ ID NO 370:
UCCAUGUACUCACCCAUCUC
Example 1G
SEQ ID NO 371:
AAAUCUGUUCAAUCAUUCAUUCAAUUUUUGGAGCAGGUUUUCUGACUUCGGUCGGA
AAACCCCU
This sequence comprises the antisense sequence (shown above
in bold) which targets the TDP-43 binding region of the TDP-43
regulated UNC13A cryptic exon.
SEQ ID NO 372:
AUCUGUUCAAUCAUUCAUUC
Example 1H
SEQ ID NO 373:
AACUCAUUCAUCUGUUCAAUCAAUUUUUGGAGCAGGUUUUCUGACUUCGGUCGGAA
AACCCCU
This sequence comprises the antisense sequence (shown above
in bold) which targets the TDP-43 binding region of the TDP-43
regulated UNC13A cryptic exon.
SEQ ID NO 374:
ACUCAUUCAUCUGUUCAAUC
Example 11
SEQ ID NO 375:
AAUCUACUCAUCACUCAUUCAUCAAUUUUUGGAGCAGGUUUUCUGACUUCGGUCGG
AAAACCCCU
This sequence comprises the antisense sequence (shown above
in bold) which targets the TDP-43 binding region of the TDP-43
regulated UNC13A cryptic exon.
SEQ ID NO 376:
UCUACUCAUCACUCAUUCAUC
Example 1K
SEQ ID NO 377:
AACCCAGGCAGCUGGAAGAGACAAUUUUUGGAGCAGGUUUUCUGACUUCGGUCGGA
AAACCCCU
This sequence comprises the antisense sequence (shown above
in bold) which targets a 3′ splice site (acceptor site) of
the TDP-43 regulated UNC13A cryptic exon.
SEQ ID NO 378:
CCCAGGCAGCUGGAAGAGAC
Example 2: Constructs targeting STMN2
Example constructs targeting the STMN2 cryptic splicing of
exon 2a were designed and have the following U7 snRNA sequences
and antisense sequences:
Example 2A
SEQ ID NO 379
AACUGUGCCACAAGCCGCAUUCAAUUUUUGGAGCAGGUUUUCUGACUUCGGUCGGA
AAACCCCU
This sequence comprises the antisense sequence (shown above in
bold) which targets an ESE in the STMN2 2a cryptic exon
SEQ ID NO 380:
CUGUGCCACAAGCCGCAUUC
Example 2B
SEQ ID NO 381:
AAGUCAACUGUGCCACAAGCCGAAUUUUUGGAGCAGGUUUUCUGACUUCGGUCGGA
AAACCCCU
This sequence comprises the antisense sequence (shown above in
bold) which targets an ESE in the STMN2 2a cryptic exon
SEQ ID NO 382:
GUCAACUGUGCCACAAGCCG
Example 2C
SEQ ID NO: 383:
AAUCCUUGUCAACUGUGCCACAAAUUUUUGGAGCAGGUUUUCUGACUUCGGUCGGA
AAACCCCU
This sequence comprises the antisense sequence (shown above in
bold) which targets an ESE in the STMN2 2a cryptic exon
SEQ ID NO 384:
UCCUUGUCAACUGUGCCACA
Example 2D
SEQ ID NO 385:
AAUAUCAUCCUUGUCAACUGUGAAUUUUUGGAGCAGGUUUUCUGACUUCGGUCGGA
AAACCCCU
This sequence comprises the antisense sequence (shown above in
bold) which targets an ESE in the STMN2 2a cryptic exon
SEQ ID NO 386:
UAUCAUCCUUGUCAACUGUG
Example 2E
SEQ ID NO: 387
AAUCCUUGUCAACUGUGCCACAAGCCAAUUUUUGGAGCAGGUUUUCUGACUUCGGU
CGGAAAACCCCU
This sequence comprises the antisense sequence (shown above in
bold) which targets an ESE in the STMN2 2a cryptic exon
SEQ ID NO 388:
UCCUUGUCAACUGUGCCACAAGCC
Example 2F
SEQ ID NO 389:
AAGUCCUGCAAUAUGAAUAUAAUUUAAUUUUUGGAGCAGGUUUUCUGACUUCGGUC
GGAAAACCCCU
This sequence comprises the antisense sequence (shown above in
bold) which targets the 3′ splice site (acceptor site) of the
STMN2 2a cryptic exon
SEQ ID NO 390:
AGUCCUGCAAUAUGAAUAUAAUUU
Example 2G
SEQ ID NO: 391
AAUGCCGAGUCCUGCAAUAUGAAUAUAAUUUUUGGAGCAGGUUUUCUGACUUCGGU
CGGAAAACCCCU
This sequence comprises the antisense sequence (shown above in
bold) which targets the 3′ splice site (acceptor site) of the
STMN2 2a cryptic exon
SEQ ID NO 392:
UGCCGAGUCCUGCAAUAUGAAUAU
Example 2H
SEQ ID NO: 393
AAUCUUCUGCCGAGUCCUGCAAUAUGAAUUUUUGGAGCAGGUUUUCUGACUUCGGU
CGGAAAACCCCU
This sequence comprises the antisense sequence (shown above in
bold) which targets the 3′ splice site (acceptor site) of the
STMN2 2a cryptic exon
SEQ ID NO 394:
UCUUCUGCCGAGUCCUGCAAUAUG
Example 21
SEQ ID NO: 395
AAGGUCUUCUGCCGAGUCCUGCAAUAAAUUUUUGGAGCAGGUUUUCUGACUUCGGU
CGGAAAACCCCU
This sequence comprises the antisense sequence (shown above in
bold) which targets the 3′ splice site (acceptor site) of the
STMN2 2a cryptic exon
SEQ ID NO 396:
GGUCUUCUGCCGAGUCCUGCAAUA
Example 2J
SEQ ID NO: 397
AACGAAGGUCUUCUGCCGAGUCCUGCAAUUUUUGGAGCAGGUUUUCUGACUUCGGU
CGGAAAACCCCU
This sequence comprises the antisense sequence (shown above in
bold) which targets the 3′ splice site (acceptor site) of the
STMN2 2a cryptic exon
SEQ ID NO 398:
CGAAGGUCUUCUGCCGAGUCCUGC
Example 2K
SEQ ID NO: 399
AACUUCUGCCGAGUCCUGCAAUAUGAAUAAUUUUUGGAGCAGGUUUUCUGACUUCG
GUCGGAAAACCCCU
This sequence comprises the antisense sequence (shown above in
bold) which targets the 3′ splice site (acceptor site) of the
STMN2 2a cryptic exon
SEQ ID NO 400:
CUUCUGCCGAGUCCUGCAAUAUGAAU
Example 2J
SEQ ID NO: 401
AAUCCUUGUCAACUGUGCCACAAGCCGCAUAAUUUUUGGAGCAGGUUUUCUGACUU
CGGUCGGAAAACCCCU.
This sequence comprises the antisense sequence (shown above in
bold) which targets the 3′ splice site (acceptor site) of the
STMN2 2a cryptic exon
SEQ ID NO 402:
UCCUUGUCAACUGUGCCACAAGCCGCAU

### Combined U7 Vector Construct Example

A combined U7 vector construct was designed with contains three U7 construct cassettes: i) one which comprises an antisense sequence complementary to UNC13A targeting the 5′ splice site; ii) one which comprises an antisense sequence complementary to STMN2 which targets a TDP-43 binding site and iii) one which comprises an antisense sequence complementary to INSR which targets a TDP-43 binding region of INSR. Each U7 construct cassette is spaced by stuffer sequences (shown below in bold). Additionally, in this combined construct, each construct comprises a hnRNP A1 protein binding domain sequence upstream of the antisense sequence.

| SEQ ID NO: | | Sequence |
|---|---|---|
| 459 | Combined U7 | GAAUUCCAACAUAGGAGCUGUGAUUGGCUGUUUUCAGCCAAUC |
| | Construct | AGCACUGACUCAUUUGCAUAGCCUUUACAAGCGGUCACAAACUC |
| | Vector | AAGAAACGAGCGGUUUUAAUAGUCUUUUAGAAUAUUGUUUAUCG |
| | | AACCGAAUAAGGAACUGUGCUUUGUGAUUCACAUAUCAGUGGA |
| | | GGGGUGUGGAAAUGGCACCUUGAUCUCACCCUCAUCGAAAGUG |
| | | GAGUUGAUGUCCUUCCCUGGCUCGCUACAGAGGCCUUUCCGCA |
| | | AUAUGAUAGGGACUUAGGGUGUCCAUGUACUCACCCAUCUCAA |
| | | UUUUUGGAGCAGGUUUUCUGACUUCGGUCGGAAAACCCCUCCC |
| | | AAGUUAACUGGUCUACAAUGAAAGCAAAACAGUUCUCUUCCCCG |
| | | CUCCCCGGUGUGUGAGAGGGGCUUUGAUCCUUCUCUGGUUUC |
| | | CUAGGAAACGCGUACGCGCCCUGUAGCGGCGCAUUAAGCGCG |
| | | GCGGGUGUGGUGGUUACGCGCAGCGUGACCGCUACACUUGCC |
| | | AGCGCCCUAGCGCCCGCUCCUUUCGCUUUCUUCCCUUCCUUUC |
| | | UCGCCACGUUCGCCGGCUUUCCCCGUCAAGCUCUAAAUCGGG |
| | | GGCUCCCUUUAGGGUUCCGAUUUAGUGCUUUACGGCACCUCG |
| | | ACCCCAAAAAACUUGAUUAGGGUGAUGGUUCACGUAGUGGGC |
| | | CAUCGCCCUGUCGACGAAUUCCAACAUAGGAGCUGUGAUUGGC |
| | | UGUUUUCAGCCAAUCAGCACUGACUCAUUUGCAUAGCCUUUACA |
| | | AGCGGUCACAAACUCAAGAAACGAGCGGUUUUAAUAGUCUUUUA |
| | | GAAUAUUGUUUAUCGAACCGAAUAAGGAACUGUGCUUUGUGAU |
| | | UCACAUAUCAGUGGAGGGGUGUGGAAAUGGCACCUUGAUCUCA |
| | | CCCUCAUCGAAAGUGGAGUUGAUGUCCUUCCCUGGCUCGCUAC |
| | | AGAGGCCUUUCCGCAAUAUGAUAGGGACUUAGGGUGCACAGAG |
| | | AGCCAAAUUCUUAAAUUUUUGGAGCAGGUUUUCUGACUUCGGU |
| | | CGGAAAACCCCUCCCAAGUUAACUGGUCUACAAUGAAAGCAAAA |
| | | CAGUUCUCUUCCCCGCUCCCCGGUGUGUGAGAGGGGCUUUGA |
| | | UCCUUCUCUGGUUUCCUAGGAAGCUUUACAAUCAACAGCAUCC |
| | | CCAUCUCUGAAGACUACAGCGUCGCCAGCGCAGCUCUCUCUA |
| | | GCGACGGCCGCAUCUUCACUGGUGUCAAUGUAUAUCAUUUUA |
| | | CUGGGGGACCUUGUGCAGAACUCGUGGUGCUGGGCACUGCUG |
| | | CUGCUGCGGCAGCUGGCAACCUGACUUGUAUCGUCGCGAUCG |
| | | GAAAUGAGAACAGGGGCAUCUUGAGCCCCUGCGGACGGUGCC |
| | | GACAGGUGCUUCUCGAUCUGCAGGAUCCGAAUUCCAACAUAGG |
| | | AGCUGUGAUUGGCUGUUUUCAGCCAAUCAGCACUGACUCAUUU |
| | | GCAUAGCCUUUACAAGCGGUCACAAACUCAAGAAACGAGCGGUU |
| | | UUAAUAGUCUUUUAGAAUAUUGUUUAUCGAACCGAAUAAGGAAC |
| | | UGUGCUUUGUGAUUCACAUAUCAGUGGAGGGGUGUGGAAAUGG |
| | | CACCUUGAUCUCACCCUCAUCGAAAGUGGAGUUGAUGUCCUUC |
| | | CCUGGCUCGCUACAGAGGCCUUUCCGCAAUAUGAUAGGGACUU |
| | | AGGGUGCCCGUAUCCGGUACUAUAUGAAUUUUUGGAGCAGGUU |
| | | UUCUGACUUCGGUCGGAAAACCCCUCCCAAGUUAACUGGUCUA |
| | | CAAUGAAAGCAAAACAGUUCUCUUCCCCGCUCCCCGGUGUGUG |
| | | AGAGGGGCUUUGAUCCUUCUCUGGUUUCCUAGGAAGAUCUAUG |
| | | GCAGCGGCCGCAUGAUAUCUAGACUGGCCUGGGCCUCAUGGGC |
| | | CUUCCGCUC |
| 460 | UNC13A | GAAUUCCAACAUAGGAGCUGUGAUUGGCUGUUUUCAGCCAAUC |
| | cassette | AGCACUGACUCAUUUGCAUAGCCUUUACAAGCGGUCACAAACUC |
| | (antisense | AAGAAACGAGCGGUUUUAAUAGUCUUUUAGAAUAUUGUUUAUCG |
| | sequence is | AACCGAAUAAGGAACUGUGCUUUGUGAUUCACAUAUCAGUGGA |
| | shown in bold, | GGGGUGUGGAAAUGGCACCUUGAUCUCACCCUCAUCGAAAGUG |
| | and hnRNP A1 | GAGUUGAUGUCCUUCCCUGGCUCGCUACAGAGGCCUUUCCGCA |
| | binding | AUAUGAUAGGGACUUAGGGUGUCCAUGUACUCACCCAUCUCAA |
| | sequence is | UUUUUGGAGCAGGUUUUCUGACUUCGGUCGGAAAACCCCUCCC |
| | shown in italics) | AAGUUAACUGGUCUACAAUGAAAGCAAAACAGUUCUCUUCCCCG |
| | | CUCCCCGGUGUGUGAGAGGGGCUUUGAUCCUUCUCUGGUUUC |
| | | CUAGGAA |
| 461 | UNC13A | UCCAUGUACUCACCCAUCUC |
| | antisense | |
| | sequence | |
| 462 | STMN2″ | GAAUUCCAACAUAGGAGCUGUGAUUGGCUGUUUUCAGCCAAUC |
| | cassette | AGCACUGACUCAUUUGCAUAGCCUUUACAAGCGGUCACAAACUC |
| | (antisense | AAGAAACGAGCGGUUUUAAUAGUCUUUUAGAAUAUUGUUUAUCG |
| | sequence is | AACCGAAUAAGGAACUGUGCUUUGUGAUUCACAUAUCAGUGGA |
| | shown in bold, | GGGGUGUGGAAAUGGCACCUUGAUCUCACCCUCAUCGAAAGUG |
| | and hnRNP A1 | GAGUUGAUGUCCUUCCCUGGCUCGCUACAGAGGCCUUUCCGCA |
| | binding | AUAUGAUAGGGACUUAGGGUGCACAGAGAGCCAAAUUCUUAAA |
| | sequence is | UUUUUGGAGCAGGUUUUCUGACUUCGGUCGGAAAACCCCUCCC |
| | shown in italics) | AAGUUAACUGGUCUACAAUGAAAGCAAAACAGUUCUCUUCCCCG |
| | | CUCCCCGGUGUGUGAGAGGGGCUUUGAUCCUUCUCUGGUUUC |
| | | CUAGGAA |
| 463 | STMN2 | CACAGAGAGCCAAAUUCUUA |
| | antisense | |
| | sequence | |
| 464 | INSR cassette | GAAUUCCAACAUAGGAGCUGUGAUUGGCUGUUUUCAGCCAAUC |
| | (antisense | AGCACUGACUCAUUUGCAUAGCCUUUACAAGCGGUCACAAACUC |
| | sequence is | AAGAAACGAGCGGUUUUAAUAGUCUUUUAGAAUAUUGUUUAUCG |
| | shown in bold, | AACCGAAUAAGGAACUGUGCUUUGUGAUUCACAUAUCAGUGGA |
| | and hnRNP A1 | GGGGUGUGGAAAUGGCACCUUGAUCUCACCCUCAUCGAAAGUG |
| | binding | GAGUUGAUGUCCUUCCCUGGCUCGCUACAGAGGCCUUUCCGCA |
| | sequence is | AUAUGAUAGGGACUUAGGGUGCCCGUAUCCGGUACUAUAUGAA |
| | shown in italics) | UUUUUGGAGCAGGUUUUCUGACUUCGGUCGGAAAACCCCUCCC |
| | | AAGUUAACUGGUCUACAAUGAAAGCAAAACAGUUCUCUUCCCCG |
| | | CUCCCCGGUGUGUGAGAGGGGCUUUGAUCCUUCUCUGGUUUC |
| | | CUAGGAAGAUCUAUGGCAGCGGCCGCAUGAUAUCUAGACUGGC |
| | | CUGGGCCUCAUGGGCCUUCCGCUC |
| 465 | INSR antisense | CCCGUAUCCGGUACUAUAUG |
| | sequence | |
| 466 | hnRNP A1 | UAUGAUAGGGACUUAGGGUG |
| | binding domain | |
| | sequence | |

### Results and Discussion

U7 smOPT Constructs Targeting UNC13A Cryptic Exon

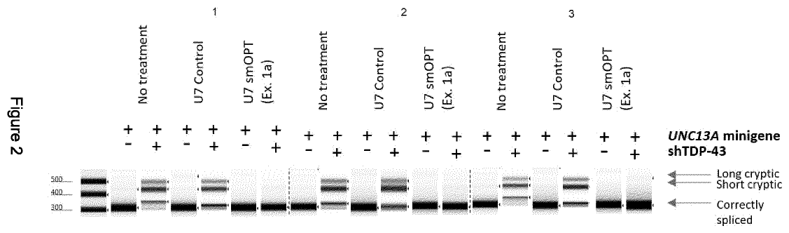

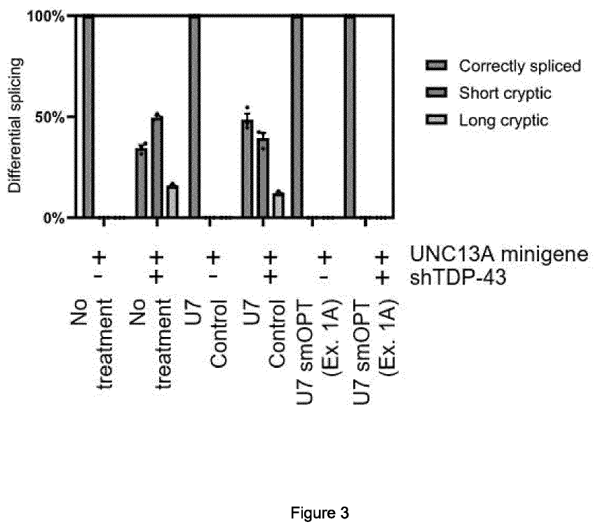

A first example construct (corresponding to Example 1A) was found to almost completely rescue splicing in SK-N-DZ cells with TDP-43 knockdown and transfected with a UNC13A minigene, as shown by RT-PCR (see FIG. 2). This was demonstrated by the presence of a band corresponding to the correctly spliced product. FIG. 3 shows the % differential splicing of the correctly spliced mature RNA (far left bar), mature RNA comprising the short UNC13A cryptic exon (middle bar) and mature RNA comprising the long UNC13A cryptic exon (far right bar) in these cells. This construct was designed to bind to the 5′-splice donor site, and provides proof of concept that targeting the splice sites of TDP-43 regulated cryptic exons can repress and restore “normal” splicing in cells depleted of TDP-43.

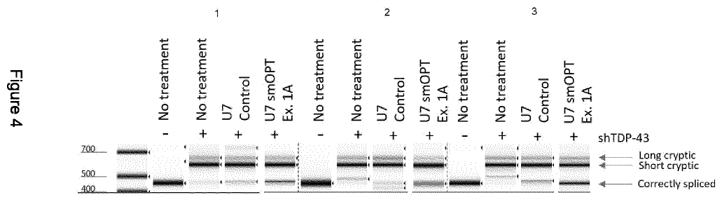

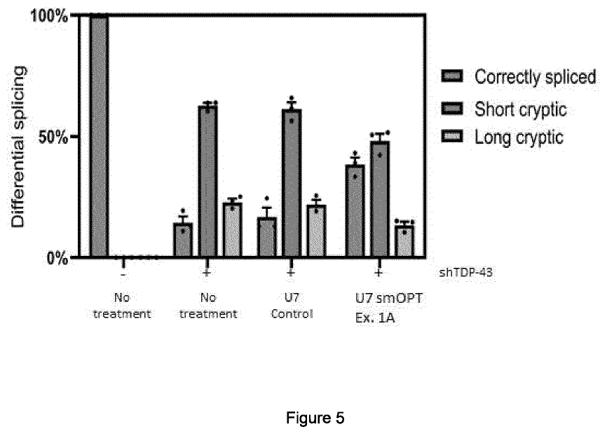

Next, experiments were performed in SH-SY5Y cells with TDP-43 knockdown with mature RNA derived from endogenous UNC13A, in cells electroporated with construct Example 1A of the invention. The construct was also found to successfully function on the endogenous RNA and rescue splicing in this system, as demonstrated by the band corresponding to the correctly spliced product in FIG. 4. FIG. 5 shows the % differential splicing of the correctly spliced mature RNA (far left bar), mature RNA comprising the short UNC13A cryptic exon (middle bar) and mature RNA comprising the long UNC13A cryptic exon (far right bar) in these cells. Differently form the minigene experiments, where the construct and minigene were delivered together with likely co-expression in cells, in these experiments we investigated the effect of our construct on endogenous transcripts, and therefore incomplete transfection leads to underestimating the splicing rescue.

Next, further example constructs (Examples 1B-1G), along with Example 1A, having different antisense sequences that targeted different splicing elements of the UNC13A cryptic exon and flanking regions thereof were tested to see if they could also rescue UNC13A cryptic exon splicing. The experiment was performed by looking at splicing of the UNC13A minigene in 293T cells with TDP-43 inducible knockdown. Correction of splicing was calculated by taking the ratios of cryptic exon containing to correctly spliced RNAs relative to control treated TDP-43 knockdown normalized to GAPDH mRNA in 293T cells.

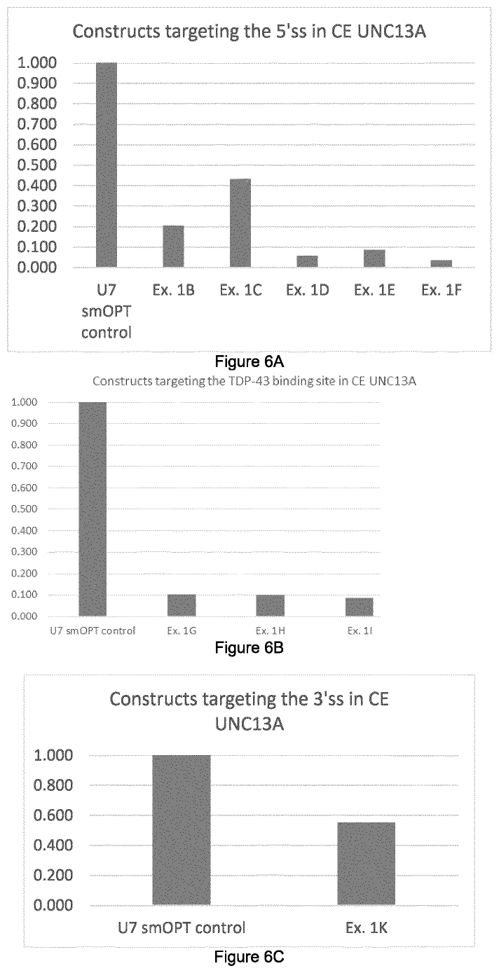

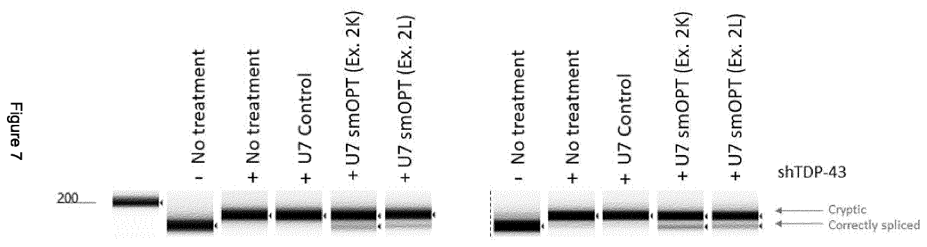

FIG. 6A demonstrates that all tested constructs that targeted the 5′-splice site of UNC13A at least partially rescued splicing; with some sequences showing more efficacy than others. Of those tested, Examples 1D-1F were found to show the best results. Interestingly, these constructs bind more of the intronic region downstream of the 5′ splice site donor and less of the exonic region upstream of the 5′ splice site donor compared to Ex. 1B and Ex1C, and hence are most likely being more efficient in interfering with the binding of the U1 snRNA to the 5′ splice site.

FIG. 6B also demonstrates that all tested constructs that targeted the TDP-43 binding site and flanking regions thereof at least partially rescued splicing.

Finally, FIG. 6C demonstrates that a construct that targets the 3′ splice site of UNC13A at least partially rescued splicing. However, the efficiency of splicing rescue was more pronounced for sequences that bound to the 5′-splice site as compared with the 3′ splice site. This may be explained by the fact that UNC13A has two 3′ splice acceptor sites. As a result, sterically blocking only one of these acceptor sites may not completely repress splicing.

U7 smOPT Constructs Targeting STMN2 2a Cryptic Exon

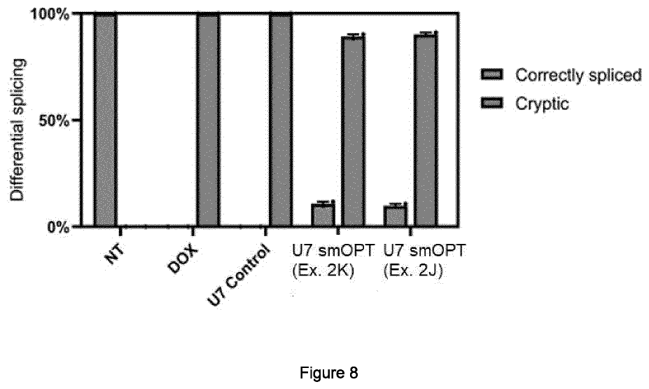

Treatment of TDP-43 depleted SH-SY5Y cells (i.e., treated with TDP-43 shRNA) was next performed with example constructs of the invention corresponding to Example 2K and 2J. These constructs comprised further antisense sequences that either targeted the 3′ splice site or STMN2a or the ESE of STMN2a. These constructs were also found to partly rescue aberrant splicing of the STMN2 cryptic exon in mature mRNA derived from endogenous STMN2. This is demonstrated in FIG. 7 by gel electrophoresis and quantification in FIG. 8 shows the differential splicing of the correctly spliced mature RNA (left bar) compared with mature RNA containing the STMN2 cryptic exon as compared with no treatment, Dox or U7 control.

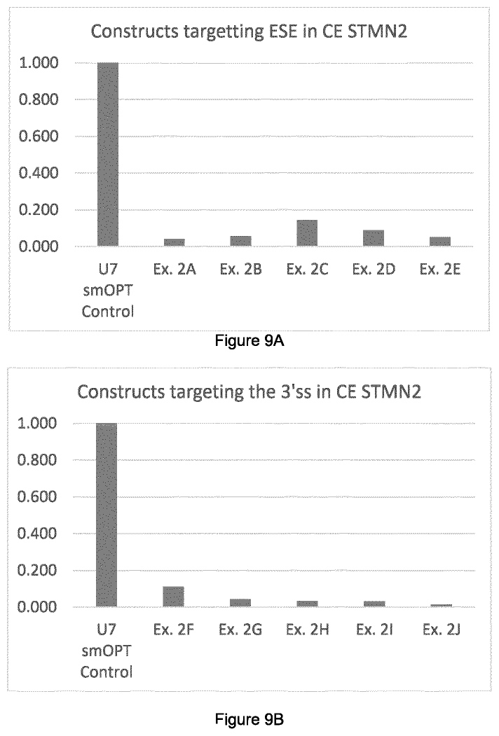

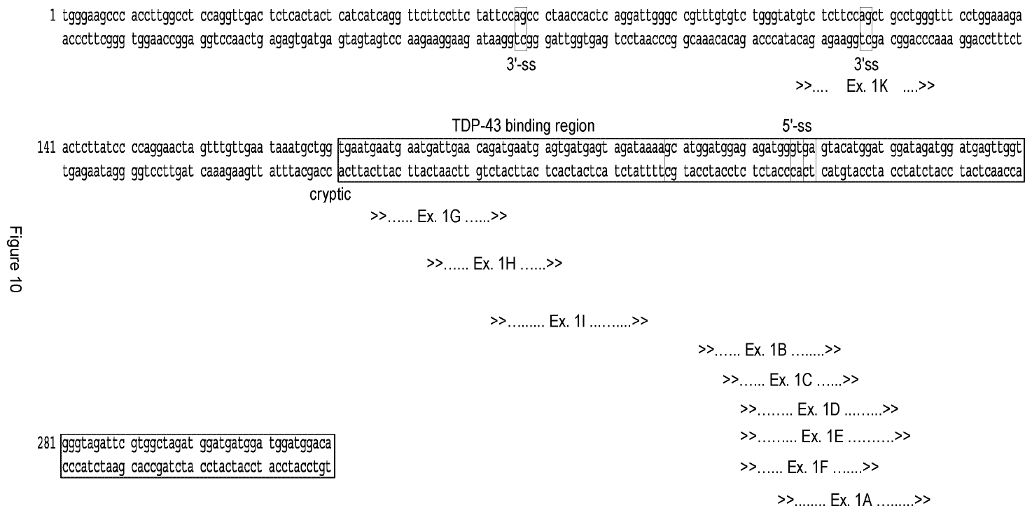

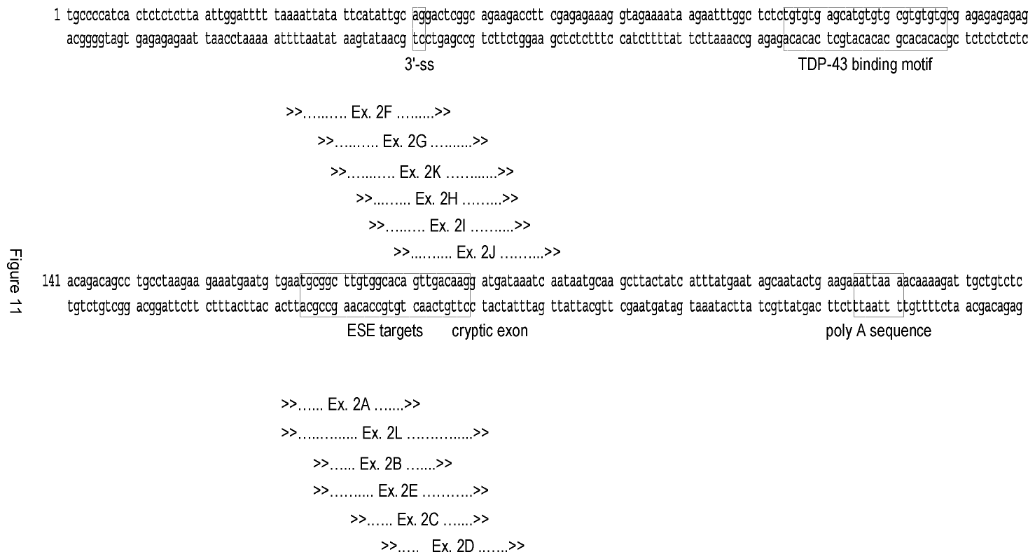

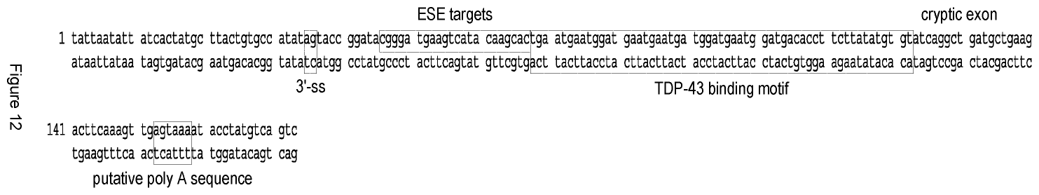

Next, further example constructs (Examples 2B-2H), along with Example 2A, having different antisense sequences that targeted different splicing elements of the STMN2 2a cryptic exon and flanking regions thereof were tested to see if they could rescue or correct splicing. The experiment was performed by looking at splicing of the STMN2 minigene in 293T cells with TDP-43 inducible knockdown. FIG. 9A demonstrates that all tested constructs that targeted exonic splice enhancers in the STMN2 cryptic exon, identified by ESE finder 3.0 in the form of SRSF binding sites, at least partially rescued splicing. Of the targets tested, Examples 2A, 2B and 2E showed the best results. Such antisense sequences are those which either completely or more effectively overlap with the ESE corresponding to the SRSF2 binding site in the STMN2 cryptic exon.

FIG. 9B further demonstrates that all tested constructs that targeted the 3′ splice site of the STMN2 cryptic exon also at least partially rescued splicing. Correction of splicing was calculated taking the ratios of cryptic exon containing to correctly spliced RNAs relative to control treated TDP-43 knockdown normalized to GAPDH mRNA in 293T cells. Notably, for constructs targeting the 3′ splice site, there was a clear trend with better efficacy shown when the part of the antisense sequence which binds to the 3′ splice site (i.e., “ag”) was closer to the 3′ end of the antisense sequence with more exonic binding of the antisense sequence, i.e., with Example 2J showing the best results.

### Further Exemplification

### Data for Endogenous Testing in SH-SY5Y Cells

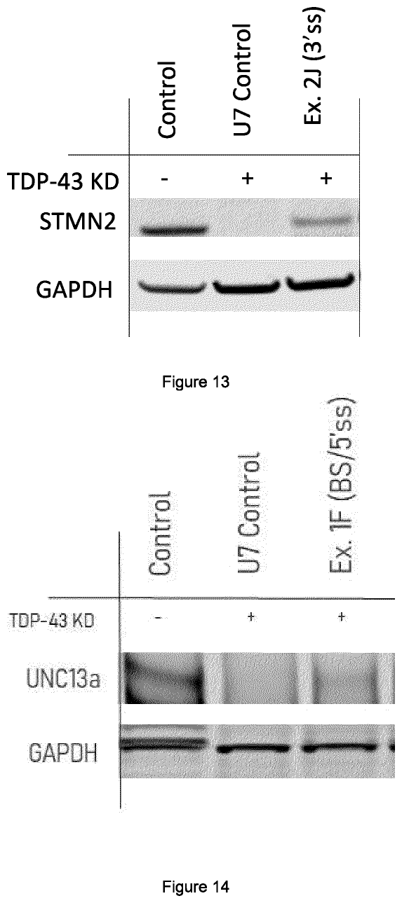

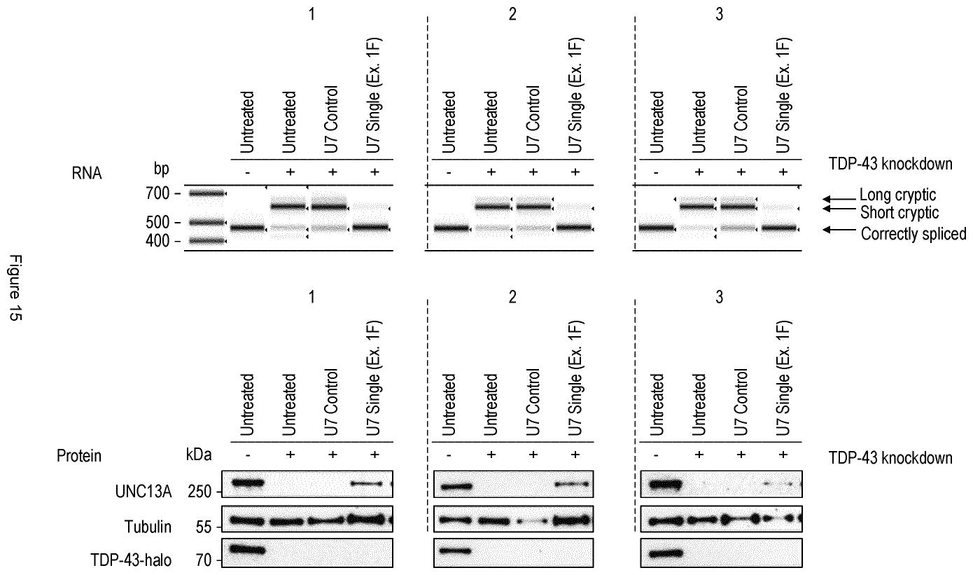

FIG. 13 shows that STMN2 levels are rescued using a construct of the invention (Example 2J) targeting the STMN2 cryptic exon in SH-SY5Y cells, while FIG. 14 analogously shows that UNC13A levels are rescued using constructs of the invention (Example 1F) to target the UNC13A cryptic exon in SH-SY57 cells. This shows that constructs of the invention work endogenously in cells.

### 13 Neuron Data

Successful RNA and protein rescue was also demonstrated in i3Neurons using a U7 constructs of the disclosure to correct mis-splicing of the TDP-43 regulated cryptic exons UNC13A, STMN2 and INSR. Human iPSC-derived cortical neurons (i3Neurons) expressing U7 constructs of the disclosure were cultured. TDP-43 knockdown was achieved by treating the cells with Halo-Protac (300 nM). RNA and protein were harvested on day 11.

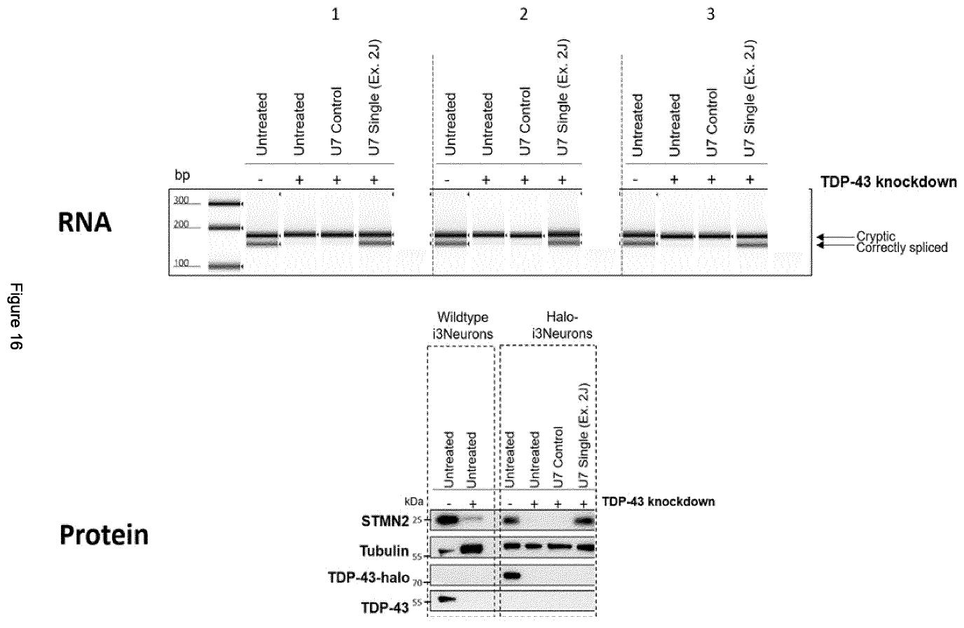

FIG. 15 Top) shows RT-PCR analysis of UNC13A splicing between exons 19 and 22 shows a rescue in splicing with Example 1F. FIG. 15 Bottom) shows western blot analysis of UNC13A levels following treatment with Example 1F. FIG. 16 Top) shows the three-primer RT-PCR analysis of STMN2 splicing at between exons 1 and 2 shows a rescue in splicing with Example 2J. FIG. 16 Bottom) shows Western blot analysis of STMN2 levels following treatment with Example 2J.

### Combined Construct Vector

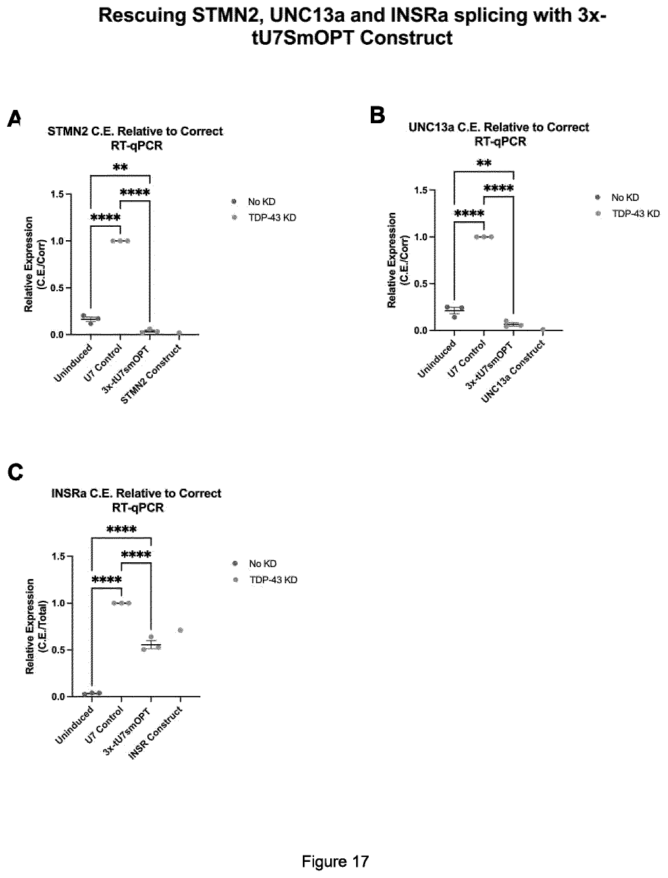

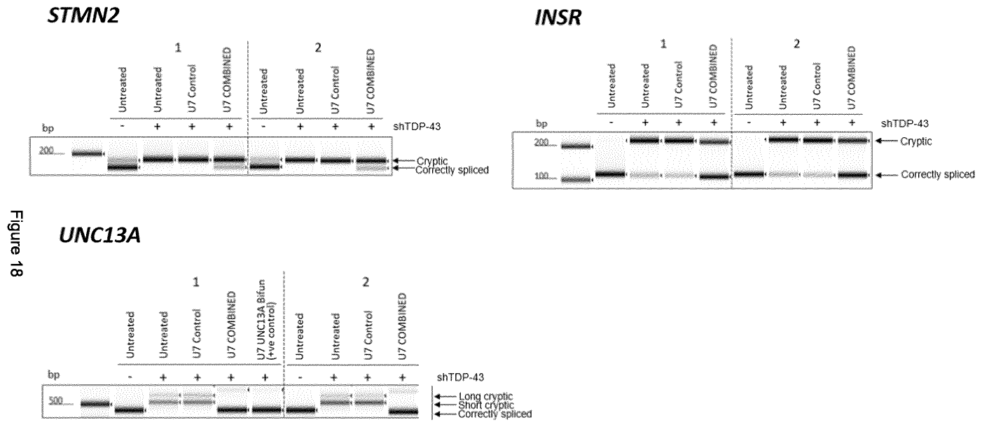

An example “multiple” construct was designed, targeting 3 different TDP-43 regulated exons: UNC13A, STMN2 and INSR. This construct is referred to herein as “3x-U7SmOPT” or “U7 Combined”. FIG. 17 shows the ratio of cryptic exon included to correctly spliced or total RT-qPCR levels of STMN2 (A), UNC13a (B) and INSR (C) mRNA in 293T-2xTDP-shRNA cells transfected with an STMN2 and an UNC13a minigene upon transfection with non-targeting control (Uninduced and U7 Control) or the combined vector pMA-3x-U7SmOPT (3x-tU7SmOPT). The combined vector 3x-tU7SmOPT construct contains three U7s in tandem (see details of sequence above), which target UNC13A, STMN2 and INSR, and is compared to CE/Correct ratios obtained upon transfection with individual constructs targeting UNC13A, STMN2 and INSR alone. Data is presented as mean ±SD relative to the ratio in non-targeting control and analyzed using ordinary one-way ANOVA with Tukey's multiple comparison test (*p<0.05, ** p<0.01, *** p<0.001, **** p<0.0001). FIG. 18 shows RNA rescue of STMN2, and INSR mis-splicing using the U7 combined construct vector in SH-SY5Y neuronal cells. TDP-43 inducible shRNA knockdown SH-SY5Y cells were left untreated or treated with doxycycline 0.025 μg/mL for 5 days. The cells were then electroporated with 2 μg of U7 DNA constructs with Ingenio Electroporation Kit (Mirus) using the A-023 setting on an Amaxa II nucleofector (Lonza). The cells were then left untreated or treated with 1 μg/mL doxycycline for 5 further days before RNA extraction on day 10. RT-PCR analysis of STMN2, INSR, and UNC13A splicing shows a rescue in splicing of all three genes using the combined triple U7 construct. The positive control demonstrated good electroporation efficiency. PCR products were resolved on a TapeStation 4200 (Agilent). The combined construct showed similar suppression of 3 TDP-43 regulated exons, UNC13A, INSR and STMN2, as compared to individual construct transfection (e.g., corresponding to constructs having SEQ ID NO: 460, 462 and 464.

Different from any prior approach, this combined construct comprises multiple antisense sequences which targets multiple cryptic exons in different genes. The result is unexpected considering the combined construct comprises multiple (and identical) promoters. This approach would not be expected to work so efficiently due to promoter competition and promoter interference. Indeed, it would be expected from previous literature that multiple promoters on one plasmid would have a different outcome to multiple plasmids with one promoter. While transcriptional interference can be prevented by cloning them in divergent orientation, this is not possible with three promoters where one promoter set will be in convergent position resulting in potential transcriptional interference.

### CONCLUSIONS

Crucially, it has been demonstrated that replacing the natural antisense sequence of a modified U7 snRNA (U7 SmOPT) with antisense sequences against splicing elements of TDP-43 regulated cryptic exons, can at least partially and effectively rescue normal splicing in TDP-43 depleted cells. This approach works by sterically blocking splicing elements that splice the cryptic exons in cells depleted with TDP-43. This approach has been demonstrated for a number of TDP-43 regulated cryptic exons, (i.e., UNC13A and STMN2), using a wide range of antisense sequences which target different splicing elements of TDP-43 regulated cryptic exons.

The constructs of the present invention are improved over alternative gene therapy approaches, such as antisense oligonucleotides, since ASOs are sensitive to degradation. As a result, ASO approaches would be less suitable as a therapy since they would need to be repeatedly delivered intrathecally and their distribution within the CNS is suboptimal. In contrast, U7 smOPT snRNA constructs can be delivered in vivo with vectorisation precluding the requirement for continuous oligo injections.

The present invention can therefore be used to further probe and understand the role of TDP-43 regulated cryptic exons in disease, while also providing promising therapeutics for diseases associated with TDP-43 pathology. Further advantages of the present invention are summarised in the statement of invention section.

### Materials and Methods

### Cloning of U7 Constructs Targeting Cryptic Exons

The U7 SmOPT expression cassettes containing the antisense sequence to the histone downstream element were ordered as gene synthesis either in pUC Simple (General Biosystems) or pMK (GeneArt, Life Technologies). To generate the constructs targeting cryptic exons, these constructs were digested with StuI and HindIII (New England Biolabs). DNA strings with 15 bp overhangs upstream and downstream of the StuI, HindIII cleavage sites containing the U7 SmOPT sequence with the antisense sequence was designed as described below and cloned into the StuI and HindIII digested U7SmOPT plasmid using InFusion Snap Assembly EcoDry Master Mix (Takara) according to the manufacturer's instructions.

Design principle of strings cloned into StuI-HindIII digested U7 SmOPT cassettes: 15 bp overhangs required for the InFusion Snap Assembly reaction are underlined, StuI and HindIII sites are shown in bold. y=antisense sequence (e.g. SEQ ID NO: 359, CUAUCCAUCCAUGUACUCACCC), SmOPT sequence is indicated in italics.

GGCUCGCUACAGAGGCCUUUCCGCAA-Y-AAUUUUUGGAGCAGGUUUUCUGA
CUUCGGUCGGAAAACCCCUCCCAAGUUAACUGGUCUACAAUGAAAGCAAAAC
AGUUCUCUUCCCCGCUCCCCGGUGUGUGAGAGGGGCUUUGAUCCUUCUCUGG
UUUCCUAGGAAACGCGUAAGCUUGUAUUCAGAU
(e.g., SEQ ID NO-403, shown with example antisense
sequence according to Example 1A).

### UNC13A and STMN Minigenes

The UNC13A minigene is described in Brown A.-L. et al, Nature, volume 603, pages 131-137 (2022). The STMN2 minigene was generated by gene synthesis. A fragment containing exon 1 and the first 300 bp of intronic sequence followed by the cryptic exon 2a preceded by 300 bp intron 1 sequence and followed by 200 bp intronic sequence followed by exon 2 preceded by 200 bp intronic sequence and followed by 200 bp intronic sequence, followed by exon 3 preceded by 200 bp intronic sequence was synthesized by GeneArt (Life Technologies). This fragment was cloned between the BamHI and XhoI sites of pcDNA3.1 (+).

Generation of inducible TDP-43 knockdown 293T cells 293T cells were cultured in DMEM/F12 medium (Gibco) with 10% tetracyclein-free FBS and 1% Penicillin/Streptomycin. Inducible 293T TDP-43 knockdown cells were generated by transfecting 80% confluent cells in a well of a 6-well plate with AAVS1-SA-puro-EF1-hspCas9 (System Biosciences) targeting the AAVS1 locus (SEQ ID NO: 404 ggggccactagggacaggat) and pAAVS1-puro 2×TDP-43 shRNA in a 1:3 ratio. pAAVS1-puro 2×TDP-43 shRNA was generated by cloning a gene synthetised fragment containing two tet-operator containing 7SK/H1 hybrid promoters expressing each one TDP-43 shRNA (target 1: SEQ ID NO: 405 GAGACTTGGTGGTGCATAA, target 2: SEQ ID NO: 406 GGAGAGGACTTGATCATTA) into the BstB1 and SaII sites of pAAV-Puro_siKD (Bertero A., et al. Current Protocols in Stem Cell Biology, 44, 5C.4.1-5C.4.48. doi: 10.1002/cpsc.45). 24 hours post transfection, cells were split into a T150 plate and subjected to selection with 0.75 ug/ml Puromycin (Gibco) for seven days, followed by a 4-day selection with 1.5 ug/ml puromycin. Single colonies were picked, expanded. The inducible TDP-43 knockdown clone was identified by qRT-PCR. Cells were induced with 1 μg/ml doxycycline for 2 days, followed by RNA isolation using the Direct-zol RNA Miniprep Plus kit (Zymo Research) and TDP-43 knockdown was validated by assessment of TDP-43 mRNA levels by comparing induced and uninduced cells by qRT-PCR with Mesa Green qPCR MasterMix (Eurogentec) according to the manufacturer's instructions using 40 ng of cDNA and 0.6 uM f.c. primers sybr TDP-43 fwd: SEQ ID NO: 407 AACCGAACAGGACCTGAAAGAG and sybr TDP-43 rev: SEQ ID NO: 408 CAGTCACACCATCGTCCATCTATC and sybr beta-actin fwd: SEQ ID NO: 409 TCCATCATGAAGTGTGACGT and sybr beta-actin rev SEQ ID NO: 429 TACTCCTGCTTGCTGATCCAC in a total volume of 20 ul using a RotorGene Q (Qiagen).

### Testing of U7SmOPT Constructs on UNC13A and STMN2 Minigenes in TDP-43 Inducible Knockdown 293T Cells

To examine the efficiency of the U7 constructs on cryptic exon splicing for STMN2 and UNC13A, 80% confluent 293T-2xTDP-shRNA cells in 6-well plates were transfected with 200 ng of STMN2 or UNC13A minigenes and 1800 ng U7SmOPT-CMV-BSD plasmids using Mirus TransIT-LT1 (Mirus Bio) according to the manufacturer's instructions. 24 hours post-transfection, cells were split 1:1 and induced with 1 ug/ml doxycycline (Sigma Aldrich). 72 hours post transfection cells were harvested and RNA was isolated using the Absolutely RNA Miniprep Kit (Agilent technologies) according to the manufacturer's instructions. RNA was reverse transcribed to cDNA using the High-capacity RNA-to-cDNA kit (Applied Biosystems). TDP-43 mRNA levels as well as the ratio of cryptic to correctly spliced levels of STMN2 and UNC13A were assessed by RT-qPCR using 20 ul final volume of PowerUp™ SYBR™ Green Master Mix (ThermoFisher) with 40 ng of cDNA, 0.3 uM f.c. primers
hTDP-43 qPCR f: TCATCCCCAAGCCATTCAGG (SEQ ID NO: 410),
hTDP-43 qPCR r: TGCTTAGGTTCGGCATTGGA (SEQ ID NO: 411),
GADPH fwd: CCAGAACATCATCCCTGCCT (SEQ ID NO: 412),
GAPDH rev: SEQ ID NO: 413 GGTCAGGTCCACCACTGACA,
UNC13A Corr f: SEQ ID NO: 414 ACCTGTCTGCATGAGAACCT,
UNC13A Cryptic f: SEQ ID NO: 415 ATGGATGGAGAGATGGAACCT,
UNC13A r: SEQ ID NO: 416 GGGCTGTCTCATCGTAGTAAAC,
STMN2 Corr f: SEQ ID NO: 417 GCTAAAACAGCAATGGCCTAC,
STMN2 Corr r: SEQ ID NO: 418 TTGCTTCACTTCCATATCATCG,
STMN2 Cryptic f: SEQ ID NO: 419 GCTAAAACAGCAATGGGACTC,
STMN2 Cryptic r: SEQ ID NO: 420 GCAGGCTGTCTGTCTCTCTC on a Rotor-Gene Q using
the fast cycling mode according to the manufacturer's instruction.

### Generation of Inducible TDP-43 Knockdown in SH-SY5Y and SK-N-DZ Cells

SH-SY5Y and SK-N-DZ cells were transduced with SmartVector lentivirus (V3IHSHEG_6494503) containing a doxycycline-inducible shRNA cassette for TDP-43. Transduced cells were selected with puromycin (1786 μg/mL) for one week.

### Testing of U7SmOPT Constructs on UNC13A and INSR Minigenes in TDP-43 Inducible Knockdown SK-N-DZ Cells

TDP-43 inducible knockdown SK-N-DZ cells were left untreated or treated with doxycycline 1 μg/mL for 3 days. The cells were then transfected with total 1 μg of DNA with a ratio of minigene to U7smOPT of 1:3 using Lipofectamine3000 (Thermofisher Scientific) and then left untreated or treated with doxycycline for 3 further days before RNA extraction on day 6. Reverse transcription was performed with RervertAid (Thermo Scientific) and cDNA was amplified by PCR with minigene-specific primers 5′-TCCTCACTCTCTGACGAGG-3′ (SEQ ID NO: 421) and 5′-CATGGCGGTCGACCTAG-3′ (SEQ ID NO: 422) for the UNC13A minigene, and primers 5′-TACCATCCACTCGACACACC-3′ (SEQ ID NO: 423) and 5′-AGTCAGTCAAGCTAGCAGAGG-3′ (SEQ ID NO: 424) for the INSR minigene. PCR products were resolved on a TapeStation 4200 (Agilent) and bands were quantified with TapeStation Systems Software v3.2 (Agilent).

### Testing of U7SmOPT Constructs on Rescue of Endogenous UNC13A and STMN2 in TDP-43 Inducible Knockdown SH-SY5Y Cells

TDP-43 inducible knockdown SH-SY5Y cells were left untreated or treated with doxycycline 0.025 μg/mL for 5 days. The cells were then electroporated with 2 μg of U7SmOPT DNA with the Ingenio Electroporation Kit (Mirus) using the A-023 setting on an Amaxa II nucleofector (Lonza). The cells were then left untreated or treated with doxycycline for 5 further days with 1 μg/mL doxycycline with a PBS wash the day after electroporation before RNA extraction on day 10. Reverse transcription was performed with RervertAid (Thermo Scientific) and cDNA was amplified by PCR with primers 5′-GACATCAAATCCCGCGTGAA-3′ (SEQ ID NO: 425) and 5′-CATTGATGTTGGCGAGCAGG-3′ (SEQ ID NO: 426) for UNC13A, and primers 5′-GCTCTCTCCGCTGCTGTAG-3′ (SEQ ID NO: 427), 5′-CGAGGTTCCGGGTAAAAGCA-3′ (SEQ ID NO: 428), and 5′-CTGTCTCTCTCTCTCGCACA-3′ (SEQ ID NO: 430) for STMN2. PCR products were resolved on a TapeStation 4200 (Agilent) and bands were quantified with TapeStation Systems Software v3.2 (Agilent).

pLVX-EF1a-mCherryT2a-BSD-U7smOPT Cloning and Virus Production

For endogenous testing in SH-SY5Y cells, U7smOPT strings were cloned into the ClaI sites of a pLVX-EF1a-mCherry T2A-BSD vector. pLVX-EF1a-mCherry T2A-BSD was generated by cloning a gene synthesized string containing the mCherry T2A-BSD ORF between the EcoRI and MluI sites of pLVx-EF1a-IRES-Puro (Clonetech Laboratories, Takara Bio) using In-Fusion Snap Assembly EcoDry (Takara Bio) following the manufacturer's instructions. The U7smOPT strings were PCR amplified from their respective U7smOPT-CMV-BSD construct with additional 15 bp overhangs using CloneAmp HiFi PCR (Clonetech Laboratories, Takara Bio) following manufacturer's instructions with use of 0.3 uM of primers LV inf pLVX Cla f: AGATCCAGTTTATCGATACCAACATAGGAGCTGTGATTGG (SEQ ID NO: 467) and LV inf pLVX Cla r: ATGAATTACTCATCGGCGAGAAAGGAAGGGAAGAAAGC (SEQ ID NO: 468) and 100 ng of template plasmid. This was then cloned into the pLVX-EF1a-mCherryT2A-BSD backbone digested with Cla1 (New England BioLabs) using In-Fusion Snap Assembly EcoDry (Takara Bio) following the manufacturer's instructions.

21 ug of the cloned pLVX-EF1a-mCherryT2A-BSD-U7smOPT and 30 ul Trans-Lentiviral Packaging Mix (Dharmacon) was transfected using Lipofectamine 2000 (Invitrogen) following manufacturer's instructions into >80% confluent HEK293T cells (Takara Bio) cultured in T-150 flasks using DMEM/F12 medium (Gibco) with 10% tetracycline-free FBS and 1% Penicillin/Streptomycin. Medium exchange was performed 24 hours post-transfection and 35 ml supernatant was harvested, filtered with 0.45 um SFCA filter (Thermo Scientific), and supplemented with LentiX Concentrator (1:4, Takara Bio) for the following two days. The mixture was then incubated overnight at 4° C., centrifuged at 1,500×g for 45 minutes at 4° C., resuspended in a total of 2 ml PBS, and aliquoted before flash freezing in LN2 and storing at −70° C. Virus titer was estimated to be at least 1×10⁷ IFU/ml using Lenti-X GoStix (Takara Bio) prior to freezing aliquots.

### Endogenous Testing of U7smOPT in TDP-43 Inducible Knockdown SH-SY5Y Cells

˜2.5 million inducible TDP-43 knockdown SH-SY5Y cells in 5 ml DMEM/F12 medium (Gibco) supplemented with 10% tetracycline-free FBS, 1% Penicillin/Streptomycin and 4 ug/ml Polybrene (SantaCruz) in T-25 flasks were transduced with 100 ul of lentivirus for 24 hours. Stable clones were selected using 2 ug/ml Blasticidin (Gibco) for 4 days followed by 2 days 1 ug/ml Puromycin (Gibco) to reselect stable clones expressing the TDP-43 shRNA cassettes. Two T-25 flasks, one for protein and one for RNA isolation, were seeded for each line and TDP-43 knockdown was induced the next day by 0.1 ug/ml doxycycline for 5 days and another 5 days of 1 ug/ml doxycycline.

### Western Blot

Total protein was harvested using cold lysis buffer [Pierce RIPA Buffer (Thermo Scientific), 2× Halt Protease Inhibitor Cocktail 100× (Thermo Scientific) 1:50, 2M MnSO4 (1:500), Cyanase Nuclease (1:1000, SERVA)] and equal amount of 2xLDL [50% NuPage LDS Sample Buffer (Invitrogen) and 50% DTT] was then added before denaturing samples for 10 minutes at 70° C. Identical quantities of denatured protein lysate were run on NuPage 4-12% Bis-Tris Gel (Invitrogen) for STMN2, and NuPage 3-8% Tris Acetate Gel (Invitrogen) for UNC13A and INSRa. Gels were then transferred in Nitrocellulose membranes (Invitrogen) and incubated in Antigen Pretreatment solution from SuperSignal™ Western Blot Enhancer Kit (Thermo Scientific) according to manufacturer instruction to enhance protein bands. After blocking with Fish Serum Blocking Buffer (Thermo Scientific), membranes were incubated at 4° C. overnight in mouse monoclonal GAPDH (1:1000, SantaCruz) and rabbit polyclonal STMN2 antibody (1:1000, Proteintech), mouse monoclonal STMN2 (1:500, R&D Systems), rabbit polyclonal Munc13-1 antibody (1:1000, Synaptic Systems) or rabbit monoclonal INSRa antibody (1:1000, Cell Signaling Technology) diluted in Primary Antibody Diluent from SuperSignal™ Western Blot Enhancer Kit (Thermo Scientific). Next, they were washed with 1×TBST and incubated with donkey anti-rabbit and anti-mouse secondary antibodies (1:10′000, Li-Cor) for 2 hours at room temperature and washed again. Finally, membranes were imaged with Odyssey CLx imaging system (Li-Cor) and protein bands quantified using Image Studio Lite (Li-Cor) by analyzing pixel density, and protein levels were normalized to GAPDH.

### RT-qPCR

RNA was extracted from SH-SY5Y cells using the Absolutely RNA Miniprep Kit (Agilent technologies) according to the manufacturer's protocol and first strand cDNA synthesis was performed with High-capacity RNA-to-cDNA kit (Applied Biosystems) or LunaScript RT SuperMix Kit (New England BioLabs). The ratio of cryptic to correctly spliced levels of STMN2 and UNC13A, cryptic to total levels of INSR as well as TDP-43 mRNA levels normalized to GAPDH were assessed by RT-qPCR using 20 ul final volume of PowerUp™ SYBR™ Green Master Mix (ThermoFisher) with 40 ng of cDNA, 0.3 uM f.c. primers SEQ ID NO: 410-420 and the following primers for the INSR gene
total INSR f: TGGGACCGCTTTACGCTTC, (SEQ ID NO: 469)
total INSR r: GAGACTGGCTGACTCGTTGAC (SEQ ID NO: 470),
CE INSR f: CTCTGGGACTGGAGCAAAC (SEQ ID NO: 471),
CE INSR r: CATCCCGTATCCGGTAAGG (SEQ ID NO: 472), on a Rotor-Gene Q using the
fast cycling mode according to the manufacturer's instruction.

mCherry T2A-BSD ORF
SEQ ID NO: 473
TATTTCCGGTGAATTCGCCGCCACCATGGTTTCCAAGGGCGAAGAGGACAACATGGCC
ATCATCAAAGAATTCATGCGGTTCAAGGTGCACATGGAAGGCAGCGTGAACGGCCACG
AGTTCGAGATTGAAGGCGAAGGCGAGGGCAGACCTTACGAGGGAACACAGACCGCCA
AGCTGAAAGTGACCAAAGGCGGCCCTCTGCCTTTTGCCTGGGACATTCTGAGCCCTCA
GTTTATGTACGGCAGCAAGGCCTACGTGAAGCACCCCGCCGATATTCCCGACTACCTG
AAGCTGAGCTTCCCCGAGGGCTTCAAGTGGGAGAGAGTGATGAACTTCGAGGACGGCG
GCGTGGTCACCGTGACTCAAGATAGCTCTCTGCAGGACGGCGAGTTCATCTACAAAGT
GAAGCTGCGGGGCACCAACTTTCCCTCTGATGGCCCCGTGATGCAGAAAAAGACCATG
GGCTGGGAAGCCAGCAGCGAGAGAATGTACCCTGAAGATGGCGCCCTGAAGGGCGAG
ATCAAGCAGCGGCTGAAACTGAAGGATGGCGGCCACTACGACGCCGAAGTGAAAACCA
CCTACAAGGCCAAGAAACCCGTGCAGCTGCCTGGCGCCTACAACGTGAACATCAAGCT
GGACATCACCAGCCACAACGAGGACTACACCATCGTGGAACAGTACGAGAGAGCCGAA
GGCAGACACAGCACAGGCGGAATGGACGAGCTGTACAAAGGCTCTGGCGAAGGCCGT
GGCAGCCTGCTTACATGCGGAGATGTGGAAGAGAACCCCGGACCTATGGCCAAGCCTC
TGAGCCAAGAGGAAAGCACCCTGATCGAAAGAGCCACCGCCACAATCAACAGCATCCC
CATCAGCGAGGATTACAGCGTGGCCTCTGCTGCCCTCAGCTCCGATGGCAGAATCTTC
ACAGGCGTGAACGTGTACCACTTCACCGGCGGACCTTGTGCCGAACTGGTGGTTCTTG
GAACAGCTGCCGCTGCCGCAGCCGGCAATCTGACATGTATTGTGGCCATCGGCAACGA
GAACCGGGGCATCCTTAGTCCTTGCGGCAGATGCAGACAGGTGCTGCTGGATCTGCAC
CCTGGCATCAAGGCCATTGTGAAGGACTCTGACGGCCAGCCTACAGCCGTGGGAATTA
GAGAGCTGCTGCCTAGCGGCTATGTGTGGGAGGGATGAACGCGTCTGGAACAAT

Generation of i3Neuron-Halo Line

The human iPSC cell line with doxycycline inducible expression of NGN2 was obtained from Michael Ward, NIH (Tian et al., 2019) and maintained following the published protocol (Fernandopulle et al., 2018). The endogenous copy of Tardbp was tagged with the HaloTag using CRISPR-Cas12 genome editing. iPSCs were nucleofected with a 4-D nucleofector (Amaxa) with the P3 Primary Cell 4-D Nucleofector kit (Amaxa V4XP-3024). One million cells were nucleofected with ribonucleoprotein complexes formed of 5 mL of Tardbp targeting crRNA (Integrated DNA Technologies 100 mM) and 20 mg of recombinant Cas12a (IDT 10001272) and 10 mg of Homology Directed Repair template (Addgene plasmid 178131). Cells were plated in Geltrex (ThermoFisher Scientific, A1413202) coated dishes in E8 Flex media (ThermoFisher Scientific, A2858501) with 1× RevitaCell (ThermoFisher Scientific, A2644501) and 1 mM HDR enhancer V2 (Integrated DNA Technologies) and maintained in a 5% CO₂ incubator at 32° C. for 24 hours. After 24 hours media was changed on cells to E8 Flex without RevitaCell or HDR enhancer and maintained in a 5% CO₂ incubator at 37° C. iPSCs were expanded and single cell plated on to Geltrex coated 96 well plates. Genomic DNA was harvested from single cell colonies and their genotype was determined by PCR amplification with primers Halo_Geno_For1 and Halo_Geno_Rev1 followed by analysis with agarose gel electrophoresis.

Tardbp crRNA:
SEQ ID NO: 474
/AITR 1/rUrArArUrUrUrCrUrArCrUrCrUrUrGrUrArGr
ArUrGrGrArArArArGrUrArArArArGrArUrGrUrCrUrGrA
rArU/AITR2/
Halo_Geno_For1:
SEQ ID NO: 475
5′-CTGGCGAGGCATCACATTTT-3′
Halo_Geno_Rev1:
SEQ ID NO: 476
5′-CGTTCTCATCTTCGGTTACCC-3′

Generation of iPSC Lines with Stable Expression of U7s

To achieve stable expression of the U7 constructs, they were delivered to iPSCs by lentiviral transduction. 50 mL of concentrated virus was delivered to 250,000 iPSCs in suspension in E8 Flex media (ThermoFisher Scientific, A2858501) with 10 mg/mL polybrene (hexadimethrine bromide, Sigma H9268) into one well of a 12-well plate following an accutase split. Cells were plated and cultured overnight. The following morning, cells were washed with PBS and media was changed to E8 Flex. Two days after lentiviral delivery, cells were selected for 48 hours with 10 mg/mL blasticidin (Sigma, SBR000221ML) iPSCs were then expanded 1-2 days before initiating neuronal differentiation. Transduction efficiency was confirmed using the fluorescence marker.

iPSC-Derived i3Neuron Differentiation and Culture

The WTC11 human iPSCs used in this study were previously engineered to express mouse or human neurogenin-2 (NGN2) under a doxycycline-inducible promoter, as well as an enzymatically dead Cas9 (+/− CAG-dCas9-BFP-KRAB) (Fernandopulle et al., 2018). These were integrated at the AAVS1 safe harbour and the CLYBL promoter safe harbour, respectively.

To initiate neuronal differentiation, 2.5 million iPSCs per 10 cm plate were single-cell plated using accutase on day 0 and re-plated onto Geltrex-coated tissue culture dishes in N2 differentiation media containing: knockout DMEM/F12 media (Life Technologies Corporation, cat. no. 12660012) with N₂ supplement (Life Technologies Corporation, cat. no. 17502048), 1× GlutaMAX (ThermoFisher Scientific, cat. no. 35050061), 1×MEM nonessential amino acids (NEAA) (ThermoFisher Scientific, cat. no. 11140050), 10 mM ROCK inhibitor (Y-27632; Selleckchem, cat. no. S1049) and 2 mg/ml doxycycline (Clontech, cat. no. 631311). Media was changed daily during this stage.

On day 3, pre-neuron cells were replated onto dishes coated with freshly made 100 mg/ml poly-D-lysine (Sigma, P7886) overnight and 10 mg/mL laminin (Thermo, cat no. 23017015) overnight in either 96-well plates (12,500-25,000 cells per well) for IncuCyte experiments, or 12-well dishes (500,000 cells per well) for RNA and protein extraction in i3Neuron Culture Media: BrainPhys media (Stemcell Technologies, cat. no. 05790) supplemented with 1× B27 Plus Supplement (ThermoFisher Scientific, cat. no. A3582801), 10 ng/ml BDNF (PeproTech, cat. no. 450-02), 10 ng/ml NT-3 (PeproTech, cat. no. 450-03), 1 mg/ml mouse laminin (Sigma, cat. no. L2020-1 MG), and 2 mg/ml doxycycline (Clontech, cat. no. 631311). On the day of plating, 1× RevitaCell (Thermo, cat no. A2644501) was added to the media. 24 hours after plating, media was fully replaced to remove RevitaCell. Following this, i3Neurons were then fed twice a week by half-media changes.

### RNA Extraction and RT-PCR

RNA was extracted from i3Neurons on day 11 and SH-SY5Y cells on day 10 using the RNeasy kit (Qiagen) or from i3Neurons on day 7 after the initiation of differentiation using a Direct-zol RNA miniprep kit (Zymo Research R2052) following the manufacturer's protocol including the on-column DNA digestion step. RNA concentrations were measured by Nanodrop and 500-1,000 ng of RNA was used for reverse transcription. First strand cDNA synthesis was performed using RevertAid (Thermo K1622) using random hexamer primers and following the manufacturer's protocol including all optional steps. cDNA was amplified by PCR with primers corresponding to SEQ ID NO: 425-426 for UNC13A, SEQ ID NO: 427, 428 and 430 for STMN2, and the following primers for INSR

SEQ ID NO: 477
INSR for: 5′-AACGACATTGCCCTGAAGAC-3′
SEQ ID NO: 478
INSR rev: 5′-CCAGTACGGCTCCCATCT-3′

PCR products were resolved on a TapeStation 4200 (Agilent).

### Western Blot

i3Neurons were lysed directly on day 11 in the sample loading buffer (Thermo NP0008). Lysates were heated at 95° C. for 5 min with 100 mM DTT. Lysates were passed through a QIAshredder (Qiagen) to shear DNA. Lysates were resolved on 4-12% Bis-Tris Gels (Thermo) and transferred to 0.45 μm PVDF (Millipore) membranes. After blocking with 5% milk, blots were probed with antibodies (Rb anti-UNC13A (Synaptic Systems 126 103) 1:2,000; Rb anti-STMN2 (ProteinTech 10586-1-AP) 1:1,000; Rb anti-INSR-α (Cell Signaling Technology #74118 clone D3U71) 1:1,000; Rb anti-INSR-β (Cell Signaling Technology #3025 clone 4B8) 1:1,000; Rat anti-Tubulin (Millipore MAB1864 clone YL1/2) 1:5,000, Mouse anti-TDP-43 (abcam ab104223 clone 3H8) 1:5,000) at 4° C. overnight. After washing, blots were probed with HRP-conjugated secondary antibodies (Goat anti-Rabbit HRP (Bio-Rad 1706515) 1:10,000; Goat anti-Mouse HRP (Bio-Rad 1706516) 1:10,000; Rabbit anti-Rat HRP (Dako P0450) 1:10,000) and developed with Chemiluminescent substrate (Merck Millipore WBKLS0500) on a ChemiDoc Imaging System (Bio-Rad).

### Generation of Inducible TDP-43 Knockdown in SH-SY5Y Neuronal Cells

SH-SY5Y cells were transduced with SmartVector lentivirus (V3IHSHEG_6494503) containing a doxycycline-inducible shRNA cassette for TDP-43. Transduced cells were selected with puromycin (1 μg/mL) for one week.

Testing of the Combined Triple U7 Construct on Rescue of Endogenous UNC13A, STMN2, and INSR Splicing in SH-SY5Y Cells with Inducible TDP-43 Knockdown

TDP-43 inducible knockdown SH-SY5Y cells were left untreated or treated with doxycycline 0.025 μg/mL for 5 days. The cells were then electroporated with 2 μg of the combined triple U7 construct and a non-targeting U7 control with the Ingenio Electroporation Kit (Mirus) using the A-023 setting on an Amaxa II nucleofector (Lonza). The cells were then left untreated or treated with doxycycline for 5 further days with 1 μg/mL doxycycline before RNA extraction on day 10.

Testing of Combined Vector 3x-U7SmOPT Constructs on STMN2 and UNC13a Minigenes in TDP-43 Inducible Knockdown 293T Cells

A combinedpMA-3x-U7smOPT vector containing three U7 cassettes against STMN2, UNC13a and INSR was ordered as gene synthesis. To examine the efficiency of the 3x-U7 constructs on cryptic exon splicing for STMN2 and UNC13A, 80% confluent 293T-2xTDP-shRNA cells in 6-well plates were transfected with 200 ng of STMN2 and 200 ng UNC13A minigenes, and 1800 ng of pMA-3x-U7smOPT plasmids using Mirus TransIT-LT1 (Mirus Bio) according to the manufacturer's instructions. 24 hours post-transfection, cells were split 1:1 and induced with 1 ug/ml doxycycline (Sigma Aldrich). 72 hours post transfection cells were harvested and RNA was isolated using the Absolutely RNA Miniprep Kit (Agilent technologies) according to the manufacturer's instructions. RNA was reverse transcribed to cDNA using the LunaScript RT SuperMix Kit (New England BioLabs). TDP-43 mRNA levels as well as the ratio of cryptic to correctly spliced levels of STMN2 and UNC13A, and ratio of cryptic to total levels of INSRa were assessed by RT-qPCR using 20 ul final volume of PowerUp™ SYBR™ Green Master Mix (ThermoFisher) with 40 ng of cDNA, 0.3 uM f.c.primers according to SEQ ID NO: 410-420 and SEQ ID NO:469-472, on a Rotor-Gene Q using the fast cycling mode according to the manufacturer's instruction.

1. A modified U7 snRNA construct comprising at least one antisense sequence having between 16 to 30 nucleotides which are at least 90% complementary to a splicing element of a TDP-43 regulated cryptic exon sequence and/or flanking regions thereof and
wherein the modified U7 snRNA construct is capable of modulating splicing of the TDP-43 regulated cryptic exon in a cell.

2. The modified U7 snRNA construct of claim 1, wherein the TDP-43 regulated cryptic exon sequence is selected from the group consisting of UNC13A, STMN2, INSR, ELAVL3, G3BP1, AARS1, CELF5, CAMK2B or UNC13B, or wherein the TDP-43 regulated cryptic exon sequence is selected from the group consisting of UNC13A, STMN2, or INSR.

3. The modified U7 snRNA construct of claim 1, wherein the flanking regions thereof are defined as the 25 nucleotides upstream or downstream of the splicing element.

4. The modified U7 snRNA construct of claim 1, comprising a sequence which is 100% complementary to a splicing element of a TDP-43 regulated cryptic exon sequence.

5. The modified U7 snRNA construct of claim 1, wherein the antisense sequence is between 16 and 26 nucleotides, or wherein the antisense sequence is between 17 and 23 nucleotides, or wherein the antisense sequence is between 18 and 22 nucleotides.

6. The modified U7 snRNA construct of claim 1, wherein the splicing element is a splice site of the cryptic exon sequence.

7. The modified U7 snRNA construct of claim 1, wherein the splicing element is an exonic splice enhancer (ESE) in the cryptic exon sequence or flanking regions thereof, wherein the ESE is as defined by ESE finder 3.0.

8. The modified U7 snRNA construct of claim 1, wherein the splicing element is a TDP-43 binding region in the cryptic exon sequence or flanking regions thereof.

9. The modified U7 snRNA construct of claim 1, wherein the U7 snRNA is an U7 smOPT construct.

10. The modified U7 snRNA construct of claim 1, wherein the TDP-43 regulated cryptic exon sequence is UNC13A, and wherein the antisense sequence is capable of binding to a splice site of the UNC13A cryptic exon, or wherein the TDP-43 regulated cryptic exon sequence is UNC13A, the antisense sequence is capable of binding to a splice site of the UNC13A cryptic exon, and the antisense sequence is at least 90% complementary to any one of SEQ ID NO: 19, SEQ ID NO: 20 or SEQ ID NO: 21 or SEQ ID NO: 22.

11. The modified U7 snRNA construct of claim 1, wherein the TDP-43 regulated cryptic exon sequence is UNC13A, and wherein the antisense sequence is capable of binding to a 5′-splice site of the UNC13A cryptic exon, or wherein the TDP-43 regulated cryptic exon sequence is UNC13A, the antisense sequence is capable of binding to a 5′-splice site of the UNC13A cryptic exon, and the antisense sequence is at least 90% complementary to any one of SEQ ID NO: 21 or 22.

12. The modified U7 snRNA construct of claim 1, wherein the TDP-43 regulated cryptic exon sequence is UNC13A, wherein the antisense sequence is capable of binding to the TDP-43 binding region of the UNC13A cryptic exon or flanking regions thereof, and or wherein the TDP-43 regulated cryptic exon sequence is UNC13A, the antisense sequence is capable of binding to the TDP-43 binding region of the UNC13A cryptic exon or flanking regions thereof and the antisense sequence is at least 90% complementary to SEQ ID NO: 23, 24 or 25.

13. The modified U7 snRNA construct of claim 1, wherein the TDP-43 regulated cryptic exon sequence is UNC13A, wherein the antisense sequence is capable of binding to one or more exonic splice enhancers (ESE) in the UNC13A cryptic exon or flanking region thereof, or wherein the TDP-43 regulated cryptic exon sequence is UNC13A, the antisense sequence is capable of binding to one or more ESE in the UNC13A cryptic exon or flanking region thereof, and the antisense sequence is at least 90% complementary to any one of SEQ ID NO: 27, 28 or 29.

14. The modified U7 snRNA construct of claim 1, wherein the TDP-43 regulated cryptic exon sequence is STMN2, and wherein the antisense sequence is capable of binding to one or more (ESE in the STMN2 cryptic exon or flanking region thereof, or wherein the TDP-43 regulated cryptic exon sequence is STMN2, the antisense sequence is capable of binding to one or more ESE in the STMN2 cryptic exon or flanking region thereof, and the antisense sequence is at least 90% complementary to any one of SEQ ID NO: 14, 15 or 16.

15. The modified U7 snRNA construct of claim 1, wherein the TDP-43 regulated cryptic exon sequence is STMN2, wherein the antisense sequence is capable of binding to the 3′-splice site of the STMN2 cryptic exon, or wherein the TDP-43 regulated cryptic exon sequence is STMN2, the antisense sequence is capable of binding to the 3′-splice site of the STMN2 cryptic exon, and the antisense sequence is at least 90% complementary to SEQ ID NO: 11.

16. The modified U7 snRNA construct of claim 1, wherein the TDP-43 regulated cryptic exon sequence is STMN2 and wherein the antisense sequence is capable of binding to the TDP-43 binding region of the STMN2 cryptic exon or flanking regions thereof, or wherein the TDP-43 regulated cryptic exon sequence is STMN2, the antisense sequence is capable of binding to the TDP-43 binding region of the STMN2 cryptic exon or flanking regions thereof, and the antisense sequence is at least 90% complementary to SEQ ID NO: 12.

17. The modified U7 snRNA construct of claim 1, wherein the TDP-43 regulated cryptic exon sequence is INSR and wherein the antisense sequence is at least 90% complementary to:
(i) the TDP-43 binding region of the INSR cryptic exon or flanking regions thereof, or the TDP-43 binding region of the INSR cryptic exon or flanking regions thereof, wherein the antisense sequence is capable of binding to SEQ ID NO: 32,
(ii) one or more ESE in the INSR cryptic exon or flanking region thereof, or one or more ESE in the INSR cryptic exon or flanking region thereof, wherein the antisense sequence is capable of binding to any one of SEQ ID NO: 34-40.
(iii) the 3′-splice site of the INSR cryptic exon, or the 3′-splice site of the INSR cryptic exon, wherein the antisense sequence is capable of binding to SEQ ID NO: 31

18. The modified U7 snRNA construct of claim 1, wherein the antisense sequence comprises a sequence with at least 90% sequence identity to SEQ ID NO 42-352, and/or wherein the antisense sequence comprises at least a 16 nucleotide sequence which has at least 90% sequence identity to at least a portion of SEQ ID NO: 359, 362, 364, 366, 368, 370, 372, 374. 376, 378, 380, 382, 384, 386, 388, 390, 392, 394, 396, 398, 400 or 402 for the same number of nucleotides.

19. A vector that comprises or encodes for the modified U7 snRNA construct of claim 1 or two or more of the modified U7 snRNA constructs.

20. The vector of claim 19, wherein the two or more modified U7 snRNA constructs comprise different antisense sequences that are capable of binding to different TDP-43 regulated cryptic exon sequences, or wherein the vector is a viral vector.

21. A pharmaceutical composition comprising one or more constructs according to claim 1, or one or more vectors comprising the one or more constructs.

22. The construct of claim 1, a vector comprising the construct, or a pharmaceutical composition comprising one or more of the constructs or the vector, for use in therapy.

23. The construct of claim 1, a vector comprising the construct, or a pharmaceutical composition comprising one or more of the constructs or the vector, for use in the treatment of a disease characterised by TDP-43 dysfunction.

24. The construct of claim 1, a vector comprising the construct, or a pharmaceutical composition comprising one or more of the constructs or the vector, for use in the treatment of a disease characterised by TDP-43 dysfunction, wherein the disease is a neurodegenerative or muscular disease.

25. The construct of claim 1, a vector comprising the construct, or a pharmaceutical composition comprising one or more of the constructs or the vector, for use in the treatment of a disease characterised by TDP-43 dysfunction, wherein the disease is Amyotrophic lateral sclerosis (ALS) and frontotemporal dementia (FTD), frontotemporal dementia (FTD), Alzheimer's disease, Inclusion body myositis/myopathy (IBM), FOSMNN (Facial onset sensory and motor neuronopathy), Perry Syndrome, Limbic-Predominant Age-Related TDP-43 Encephalopathy (LATE) or a combination thereof.

26. A method of correcting splicing of a TDP-43 regulated cryptic exon in a cell, the method comprising delivering to a cell the construct of claim 1, a vector comprising the construct, or a pharmaceutical composition comprising one or more of the constructs or the vector, wherein the method comprises contacting the construct with a cell, wherein the construct modulates splicing of the TDP-43 regulated cryptic exon.
